(19) United States
(12) Patent Application Publication
(10) Pub. No.: US 2023/0355524 A1
(43) Pub. Date: Nov. 9, 2023

(54) METHODS AND COMPOSITIONS FOR DELIVERING MRNA CODED ANTIBODIES

(71) Applicant: TRANSLATE BIO, INC., Waltham, MA (US)

(72) Inventors: Lianne BOEGLIN, Hollis, NH (US); Christopher GUENARD, Lexington, MA (US); Khang Anh TRAN, Lexington, MA (US); Anusha DIAS, Lexington, MA (US); Frank DEROSA, Chelmsford, MA (US)

(21) Appl. No.: 18/352,615

(22) Filed: Jul. 14, 2023

Related U.S. Application Data
(63) Continuation of application No. PCT/US2022/012416, filed on Jan. 14, 2022.
(60) Provisional application No. 63/137,528, filed on Jan. 14, 2021.

(51) Int. Cl. A61K 9/107; C07K 16/28; A61K 48/00; A61K 9/00

(52) U.S. Cl. CPC A61K 9/1075; C07K 16/2866; A61K 48/0033; A61K 9/007; A61K 2039/505

(57) ABSTRACT

The present invention provides, among other things, method of treating an immune disease in a subject, the method comprising: administering to a subject in need thereof one or more mRNAs encoding a heavy chain and a light chain of an antibody that binds to a protein target selected from IL-4, IL-5, IL-6, IL-9, IL-13, IL-25, or IL-33, IL-4R, IL-5R, IL-6R, IL-9R, IL-13R, IL-25R or IL-33R, and wherein the one or more mRNAs are encapsulated in a lipid nanoparticle (LNP). The present invention also provides, among other things, a composition comprising one or more mRNAs encoding a heavy chain and a light chain of an antibody that binds a protein target selected from IL-4, IL-5, IL-6, IL-9, IL-13, IL-25, or IL-33, IL-4R, IL-5R, IL-6R, IL-9R, IL-13R, IL-25R or IL-33R, and wherein the one or more mRNAs are encapsulated in a lipid nanoparticle (LNP).

### CROSS-REFERENCE TO RELATED APPLICATIONS

This application is a continuation of International Application No. PCT/US2022/012416, filed on Jan. 14, 2022, which claims priority to and benefit of U.S. Provisional Application No. 63/137,528, filed on Jan. 14, 2021. The contents of each of the foregoing applications are hereby incorporated by reference in their entireties.

### INCORPORATION BY REFERENCE OF SEQUENCE LISTING

The content of the text file named, “MRT-2210US1_SL.xml”, which was created on Jul. 13, 2023, and is 59,814 bytes in size, is hereby incorporated by reference in its entirety.

### BACKGROUND

Antibodies have powerful therapeutic effects and are currently used for the treatment of a range of diseases including cancer, immune diseases, cardiovascular disease, and transplant rejection. Traditionally, therapeutic antibodies are produced by recombinant technology, formulated and then administered to patients in need of antibody therapy. However, antibody production and formulation is highly expensive. In addition, many antibodies only have a very short half-life in vivo and therefore, may not reach their target antigen or target tissue before being degraded. To achieve desired efficacy, antibody therapy often requires high doses and frequent administration, which can lead to unwanted, off-target effects.

### SUMMARY OF THE INVENTION

The present invention provides, among other things, a method of treating disease in a subject. In some aspects, the present invention provides a method of treating an immune disease in a subject. In some embodiments, the method involves administering to the subject a composition comprising mRNA that encodes an antibody that targets drivers of Type 2 inflammation, such targets including, for example, IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R). In some embodiments, the method involves administering to the subject a composition comprising mRNA that encodes an antibody that binds and/or inhibits drivers of Type 2 inflammation, such targets including, for example, IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R). The inventors have surprisingly discovered robust and efficient means to deliver mRNA encoding antibodies encapsulated in lipid nanoparticles (LNP) that target specific cytokines involved in immune disease. The methods and compositions disclosed herein can be used to treat various disease, such as those that are associated with lung-associated immune disease. Examples of such lung-associated immune diseases include asthma, chronic rhinosinusitis with nasal polyps (CRSwNP), chronic obstructive pulmonary disease (COPD), systemic sclerosis—interstitial lung disease (SSc-ILD), idiopathic pulmonary fibrosis IPF, sarcoidosis, or allergy. The inventors have also surprisingly discovered that administration of the herein described compositions via inhalation or nebulization resulted in low to no systemic exposure, thus potentially avoiding unwanted systemic side-effects.

In some aspects, a method of treating an immune disease in a subject, the method comprising: administering to a subject in need thereof one or more mRNAs encoding a heavy chain and a light chain of an antibody that binds and/or inhibits a protein target selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1) and wherein the one or more mRNAs are encapsulated in a lipid nanoparticle (LNP). In some embodiments, the protein target is IL-4. In some embodiments, the protein target is IL-5. In some embodiments, the protein target is IL-6. In some embodiments, the protein target is IL-9. In some embodiments, the protein target is IL-13. In some embodiments, the protein target is IL-25. In some embodiments, the protein target is IL-33. In some embodiments, the protein target is IL-4 Receptor (IL-4R, e.g., IL-4Rα). In some embodiments, the protein target is IL-5 Receptor (IL-5R). In some embodiments, the protein target is IL-6 Receptor (IL-6R). In some embodiments, the protein target is IL-9 Receptor (IL-9R). In some embodiments, the protein target is IL-13 Receptor (IL-13R). In some embodiments, the protein target is IL-25 Receptor (IL-25R). In some embodiments, the protein target is IL-33 Receptor (IL-33R, e.g. ST2, also known as IL1RL1).

In some aspects, provided herein is a method of treating an immune disease in a subject, the method comprising: administering to a subject in need thereof one or more mRNAs encoding a heavy chain and a light chain of an antibody that binds and/or inhibits a protein selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP)._In some embodiments, the antibody binds and/or inhibits IL-4. In some embodiments, the antibody binds and/or inhibits IL-5. In some embodiments, the antibody binds and/or inhibits IL-6. In some embodiments, the antibody binds and/or inhibits IL-9. In some embodiments, the antibody binds and/or inhibits IL-13. In some embodiments, the antibody binds and/or inhibits IL-25. In some embodiments, the antibody binds and/or inhibits IL-33. In some embodiments, the antibody binds and/or inhibits IL-4 Receptor (IL-4R, e.g., IL-4Rα). In some embodiments, the antibody binds and/or inhibits IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1).

In some embodiments, the antibody is an anti-IL6R antibody or an anti-IL4Rα antibody. In some embodiments, the antibody is an anti-IL6R antibody. In some embodiments, the antibody is an anti-IL4Rα antibody.

In some embodiments, the immune disease is associated with an increase in type 2 inflammation associated-cytokines.

In some embodiments, the immune disease is selected from asthma, chronic rhinosinusitis with nasal polyps (CRSwNP), chronic obstructive pulmonary disease (COPD), systemic sclerosis—interstitial lung disease (SSc-ILD), idiopathic pulmonary fibrosis IPF, sarcoidosis, or allergy. In some embodiments, the immune disease is asthma. In some embodiments, the immune disease is chronic rhinosinusitis with nasal polyps (CRSwNP). In some embodiments, the immune disease is chronic obstructive pulmonary disease (COPD). In some embodiments, the immune disease is systemic sclerosis—interstitial lung disease (SSc-ILD). In some embodiments, the immune disease is idiopathic pulmonary fibrosis IPF. In some embodiments, the immune disease is sarcoidosis. In some embodiments, the immune disease is allergy.

In some embodiments, the administering is performed by nebulization, intratracheal delivery, or inhalation. In some embodiments, the administering is performed by nebulization. In some embodiments, pulmonary delivery is via nebulization of the compound using a nebulizer, preferably a mesh nebulizer. In some embodiments, the nebulizer delivers the compound to lung cells in the form of an aerosol. In some embodiments, the lung cells are lung epithelial cells. In some embodiments, compositions with lipid nanoparticles having an average size of about 50-70 nm are particularly suitable for pulmonary delivery via nebulization.

In some embodiments, the administration is performed by intratracheal delivery. In some embodiments, the administration is performed by inhalation.

In some embodiments, the administering results in administration of the mRNA to lung tissue.

In some embodiments, the administering results in antibody expression for at least about 48 hours, 72 hours, 96 hours, or 120 hours. Accordingly, in some embodiments, the administering results in antibody expression for at least about 48 hours. In some embodiments, the administering results in antibody expression for at least about 72 hours. In some embodiments, the administering results in antibody expression for at least about 96 hours. In some embodiments, the administering results in antibody expression for at least about 120 hours. In some embodiments, the administering results in antibody expression for between about 72 and 120 hours. In some embodiments, the administering results in antibody expression for between 96 and 120 hours.

In some embodiments, low or no systemic exposure of the mRNA occurs following administration of the composition to the lung of the subject.

In some embodiments, the immune disease is selected from autoimmune dermatitis or atopic dermatitis. Accordingly, in some embodiments, the immune disease is autoimmune dermatitis. In some embodiments, the immune disease is atopic dermatitis.

In some embodiments, the administering is performed intravenously. In some embodiments, the administering is performed intraperitoneally. In some embodiments, the antibody is expressed systemically, for example, when administered intravenously or intraperitoneally.

In some embodiments, the LNP comprises one or more of cationic lipid, non-cationic lipid, and PEG-modified lipids.

In some embodiments, the LNP further comprises cholesterol.

In some embodiments, the LNP has a molar ratio of cationic lipid(s) to non-cationic lipid(s) to PEG-modified lipid(s) between about 30-60:25-35: 1-15, respectively.

In some embodiments, the non-cationic lipid is selected from 1,2-Dierucoyl-sn-glycero-3-phosphoethanolamine (DEPE), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

In some embodiments, the LNP comprises DMG-PEG-2000, Guan-SS-Chol, and DOPE. In some embodiments, the LNP comprises DMG-PEG-2000. In some embodiments, the LNP comprises Guan-SS-Chol. In some embodiments, the LNP comprises DOPE.

In some embodiments, in the DMG-PEG-2000, Guan-SS-Chol, and DOPE are present at a ratio of about 1-15:30-60:25-35. In some embodiments, the DMG-PEG-2000, Guan-SS-Chol, and DOPE are present at a ratio of about 5:60:35.

In some embodiments, the heavy chain and the light chain are encoded in a single mRNA.

In some embodiments, the heavy chain and the light chain are encoded in separate mRNAs.

In some embodiments, the LNP has a size of no greater than 150 nm.

In some embodiments, the LNP has a size of no greater than 100 nm.

In some embodiments, the LNP has a size of no greater than 75 nm.

In some embodiments, the LNP has a size of about 60 nm.

In some embodiments, the one or more mRNAs are modified to enhance stability.

In some embodiments, the one or more mRNAs are modified to include a modified nucleotide, a cap structure, a poly A tail, a 5′ and/or 3′ untranslated region.

In some embodiments, the one or more mRNAs are unmodified.

In some aspects, a composition is provided comprising one or more mRNAs encoding a heavy chain and a light chain of an antibody that binds and/or inhibits a protein target selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP). In some embodiments, the protein target is IL-4. In some embodiments, the protein target is IL-5. In some embodiments, the protein target is IL-6. In some embodiments, the protein target is IL-9. In some embodiments, the protein target is IL-13. In some embodiments, the protein target is IL-25. In some embodiments, the protein target is IL-33. In some embodiments, the protein target is IL-4 Receptor (IL-4R, e.g., IL-4Rα). In some embodiments, the protein target is IL-5 Receptor (IL-5R). In some embodiments, the protein target is IL-6 Receptor (IL-6R). In some embodiments, the protein target is IL-9 Receptor (IL-9R). In some embodiments, the protein target is IL-13 Receptor (IL-13R). In some embodiments, the protein target is IL-25 Receptor (IL-25R). In some embodiments, the protein target is IL-33 Receptor (IL-33R, e.g. ST2, also known as IL1RL1).

In some embodiments, the antibody is an anti-IL6R antibody or an anti-IL4Rα antibody. In some embodiments, the antibody is an anti-IL6R antibody. In some embodiments, the antibody is an anti-IL4Rα antibody.

In some embodiments, wherein the LNP comprises one or more of cationic lipid, non-cationic lipid, and PEG-modified lipids.

In some embodiments, the LNP further comprises cholesterol.

In some embodiments, the LNP has a molar ratio of cationic lipid(s) to non-cationic lipid(s) to PEG-modified lipid(s) between about 30-60:25-35:1-15, respectively.

In some embodiments, the non-cationic lipid is selected from 1,2-Dierucoyl-sn-glycero-3-phosphoethanolamine (DEPE), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

In some embodiments, the LNP comprises DMG-PEG-2000, Guan-SS-Chol, and DOPE.

In some embodiments, in the DMG-PEG-2000, Guan-SS-Chol, and DOPE are present at a ratio of about 1-15:30-60:25-35. In some embodiments, in the DMG-PEG-2000, Guan-SS-Chol, and DOPE are present at a ratio of about 5:60:35.

In some embodiments, the mRNA encodes an anti-IL6R antibody heavy chain comprising a sequence at least 80% identical to EVQLVESGGGLVQPGRSLRLSCAASRFTFDDYAMHWVRQAPGKGLEWVSGISWNSGRI GYADSVKGRFTISRDNAENSLFLQMNGLRAEDTALYYCAKGRDSFDIWGQGTMVTVSS ASTKGPSVFPLAPSSKSTSGGTAALGCLVKDYFPEPVTVSWNSGALTSGVHTFPAVLQSS GLYSLSSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY NSTYRVVSVLTVLHQDWLNGKEYKCKVSNKALPAPIEKTISKAKGQPREPQVTYLPPSR DELTKNQVSLTCLVKGFYPSDIAVEWESNGQPENNYKTTPPVLDSDGSFFLYSKLTVDK SRWQQGNVFSCSVMHEALHNHYTQKSLSLSPGK (SEQ ID NO: 18). In some embodiments, the mRNA encodes an anti-IL6R antibody heavy chain comprising a sequence 70%, 75%, 80%, 85%, 90%, 95% or more identical to SEQ ID NO: 18. In some embodiments, the mRNA encodes an anti-IL6R antibody heavy chain comprising a sequence identical to SEQ ID NO: 18.

In some embodiments, the mRNA encodes an anti-IL6R antibody heavy chain further comprising a secretion sequence at least 80% identical to: MATGSRTSLLLAFGLLCLPWLQEGSAFPTIPLS (SEQ ID NO: 26). In some embodiments, the mRNA encodes an anti-IL6R antibody heavy chain further comprising a secretion sequence 70%, 75%, 80%, 85%, 90%, 95% or more identical to SEQ ID NO: 26. In some embodiments, the mRNA encodes an anti-IL6R antibody heavy chain further comprising a secretion sequence to SEQ ID NO: 26.

In some embodiments, the mRNA encodes an anti-IL6R antibody light chain comprising a sequence at least 80% identical to:

DIQMTQSPSSVSASVGDRVTITCRASQGISSWLAWYQQKPGKAPKLLIYGASSLESGVPS RFSGSGSGTDFTLTISSLQPEDFASYYCQQANSFPYTFGQGTKLEIKRTVAAPSVFIFPPSD EQLKSGTASVVCLLNNFYPREAKVQWKVDNALQSGNSQESVTEQDSKDSTYSLSSTLTL SKADYEKHKVYACEVTHQGLSSPVTKSFNRGEC (SEQ ID NO: 19). In some embodiments, the mRNA encodes an anti-IL6R antibody light chain comprising a sequence 70%, 75%, 80%, 85%, 90%, 95% or more identical to SEQ ID NO: 19. In some embodiments, the mRNA encodes an anti-IL6R antibody light chain comprising a sequence identical to SEQ ID NO: 19.

In some embodiments, the mRNA encodes an anti-IL6R antibody light chain further comprising a secretion sequence at least 80% identical to: MATGSRTSLLLAFGLLCLPWLQEGSAFPTIPLS (SEQ ID NO: 26). In some embodiments, the mRNA encodes an anti-IL6R antibody light chain further comprising a secretion sequence 70%, 75%, 80%, 85%, 90%, 95% or more identical to SEQ ID NO: 26. In some embodiments, the mRNA encodes an anti-IL6R antibody light chain further comprising a secretion sequence to SEQ ID NO: 26.

In some embodiments, the mRNA that encodes the anti-IL6R antibody heavy chain is codon optimized and comprises a sequence at least 80% identical to one of the following sequences:

(A)
(SEQ ID NO: 2)
ATGGCCACTGGAAGCCGGACAAGCCTGCTGCTGGCCTTTG
GCCTGCTGTGTCTGCCTTGGCTGCAGGAGGGAAGCGCATT
TCCAACAATTCCTCTGAGCGAAGTGCAGCTGGTGGAGTCT
GGAGGAGGCCTCGTGCAGCCAGGCAGATCCCTGAGGCTCT
CCTGCGCCGCTAGCAGATTCACTTTCGACGACTACGCCAT
GCACTGGGTGCGGCAGGCACCTGGCAAGGGACTGGAATGG
GTGTCCGGCATTTCTTGGAACAGCGGGCGGATCGGGTACG
CGGACAGCGTGAAAGGAAGGTTTACAATCTCCCGGGACAA
TGCTGAGAACTCCCTGTTCCTGCAGATGAACGGCCTGAGA
GCTGAAGACACAGCACTGTACTATTGCGCAAAAGGCCGCG
ACTCCTTTGACATCTGGGGGCAGGGCACAATGGTGACCGT
GTCTAGCGCCTCCACAAAAGGACCTAGCGTTTTCCCACTG
GCTCCATCTAGCAAGTCTACATCCGGGGGCACCGCCGCTC
TGGGCTGTCTGGTGAAGGATTACTTCCCTGAGCCCGTCAC
TGTCAGCTGGAACTCCGGAGCTCTGACCTCAGGCGTGCAC
ACTTTTCCCGCTGTGCTGCAGAGCTCTGGCCTGTACAGCC
TGAGCAGCGTTGTGACCGTGCCTAGCTCATCCCTCGGCAC
CCAGACCTATATCTGCAACGTCAACCACAAACCTTCCAAC
ACCAAAGTGGACAAGAAAGTGGAACCTAAGTCCTGCGATA
AGACTCATACTTGCCCTCCTTGTCCAGCACCAGAGCTGCT
GGGGGGGCCAAGCGTGTTTCTCTTTCCACCTAAGCCTAAA
GACACCCTGATGATCTCCAGGACCCCAGAGGTGACATGTG
TGGTGGTGGACGTGTCTCATGAGGACCCTGAGGTGAAATT
CAATTGGTATGTGGACGGCGTTGAGGTTCACAACGCAAAG
ACCAAGCCAAGGGAGGAGCAGTATAATAGCACCTATCGCG
TGGTGTCCGTCCTGACAGTGCTGCACCAGGACTGGCTGAA
CGGGAAGGAGTATAAGTGTAAAGTGAGCAACAAGGCACTG
CCTGCTCCTATCGAGAAGACTATCAGCAAAGCTAAAGGAC
AGCCAAGAGAGCCCCAGGTGACCTACCTGCCACCTTCTCG
GGACGAACTGACCAAAAACCAGGTGAGCCTGACTTGCCTG
GTGAAGGGCTTTTATCCCTCTGATATTGCAGTGGAGTGGG
AGAGTAACGGGCAGCCCGAGAACAACTACAAGACTACTCC
ACCAGTTCTGGATTCCGACGGCAGCTTCTTCCTGTATAGC
AAACTGACAGTGGACAAGTCCCGGTGGCAGCAGGGCAACG
TTTTTAGCTGCAGCGTGATGCATGAGGCTCTGCACAACCA
TTACACACAGAAGTCTCTGTCTCTGTCCCCCGGAAAGTGA;
(B)
(SEQ ID NO: 3)
ATGGCCACCGGGTCTCGGACAAGCCTCCTGCTCGCATTCG
GGCTCCTGTGTCTGCCTTGGCTGCAAGAAGGATCCGCATT
TCCCACCATTCCACTGTCTGAGGTGCAGCTGGTCGAGTCT
GGAGGAGGACTGGTGCAGCCTGGCAGGTCTCTGAGGCTGT
CTTGCGCTGCCAGCCGGTTTACCTTTGATGATTACGCCAT
GCACTGGGTGAGGCAGGCTCCCGGCAAAGGACTGGAATGG
GTGTCCGGAATTTCCTGGAATAGTGGCAGGATCGGCTATG
CCGACTCTGTCAAAGGCCGGTTTACAATCTCCCGCGACAA
CGCTGAGAACTCCCTGTTCCTGCAGATGAACGGCCTGAGA
GCTGAAGATACCGCCCTGTACTATTGCGCCAAGGGGCGCG
ACAGCTTCGACATTTGGGGCCAGGGAACCATGGTGACTGT
GAGCAGCGCATCCACAAAAGGGCCCTCCGTGTTCCCCCTG
GCACCTTCCAGTAAATCCACTTCTGGCGGAACAGCAGCTC
TCGGCTGTCTGGTGAAGGATTATTTCCCCGAGCCAGTGAC
AGTGTCTTGGAATTCTGGCGCACTCACCAGTGGAGTCCAC
ACTTTTCCAGCCGTGCTGCAGAGCTCCGGACTGTATTCCC
TGAGCTCCGTCGTGACAGTGCCATCCTCTTCTCTGGGAAC
TCAGACATATATTTGCAACGTTAATCATAAGCCTTCTAAC
ACCAAGGTGGATAAGAAAGTGGAGCCCAAATCCTGCGACA
AGACACACACTTGCCCACCATGCCCTGCCCCTGAACTGCT
GGGAGGACCAAGCGTGTTTCTCTTCCCTCCTAAGCCTAAG
GATACCCTGATGATCTCTAGGACCCCAGAGGTGACATGCG
TGGTGGTTGACGTCTCCCATGAAGATCCTGAAGTGAAATT
TAACTGGTACGTGGACGGAGTGGAAGTGCACAATGCAAAG
ACCAAACCCCGCGAGGAACAGTACAACTCCACTTACCGGG
TGGTTTCCGTGCTGACAGTGCTGCACCAGGATTGGCTGAA
CGGAAAAGAGTATAAGTGCAAAGTGAGCAATAAGGCCCTG
CCCGCCCCTATTGAGAAGACCATCTCTAAGGCTAAAGGCC
AGCCTCGCGAACCCCAGGTTACCTATCTGCCTCCAAGCAG
AGATGAGCTCACCAAAAACCAGGTGTCTCTGACCTGTCTG
GTGAAAGGCTTCTATCCAAGCGATATCGCCGTGGAGTGGG
AGTCCAACGGACAGCCAGAAAACAATTACAAGACTACCCC
ACCTGTCCTGGACAGCGACGGGAGCTTCTTTCTGTACTCT
AAGCTGACAGTCGACAAAAGCCGGTGGCAGCAAGGCAACG
TCTTCAGCTGCAGCGTCATGCACGAGGCCCTGCATAATCA
TTATACTCAGAAGTCTCTGAGCCTGAGCCCTGGCAAGTAG;
(C)
(SEQ ID NO: 4)
ATGGCCACTGGAAGCAGAACCTCCCTGCTGCTGGCATTCG
GACTGCTGTGCCTGCCATGGCTGCAGGAGGGATCCGCTTT
CCCAACCATCCCCCTCAGCGAGGTGCAGCTCGTTGAATCT
GGAGGAGGACTGGTGCAACCAGGACGCTCCCTGAGACTGT
CTTGTGCTGCTTCCAGGTTTACTTTTGACGATTATGCTAT
GCACTGGGTGAGACAGGCCCCAGGAAAAGGACTGGAATGG
GTGTCTGGAATTTCTTGGAACAGCGGACGCATTGGCTACG
CCGACTCTGTGAAGGGAAGGTTTACTATCTCCAGGGATAA
CGCGGAAAACTCCCTCTTCCTCCAGATGAACGGCCTGAGG
GCAGAGGACACCGCTCTGTACTACTGCGCCAAAGGAAGAG
ATAGCTTCGATATCTGGGGACAGGGGACCATGGTGACAGT
TTCCAGCGCTAGCACCAAGGGCCCCAGCGTGTTCCCACTG
GCCCCATCCTCCAAGAGCACTTCTGGCGGGACTGCTGCAC
TGGGCTGCCTGGTGAAGGATTATTTCCCTGAGCCTGTGAC
AGTGAGCTGGAACTCAGGAGCACTGACTTCCGGGGTGCAT
ACATTCCCCGCTGTGCTGCAGTCTTCTGGGCTGTATTCCC
TCAGCAGCGTGGTGACCGTCCCTTCCTCAAGCCTGGGAAC
CCAGACATATATTTGTAACGTGAACCACAAGCCAAGCAAT
ACAAAGGTGGATAAGAAGGTGGAGCCTAAGTCCTGTGACA
AAACACACACATGTCCCCCATGTCCAGCTCCTGAACTGCT
TGGCGGACCATCCGTCTTCCTGTTTCCACCCAAACCAAAG
GATACACTGATGATCAGCCGGACACCAGAGGTGACTTGCG
TCGTCGTGGACGTCAGCCATGAAGACCCCGAGGTGAAGTT
TAATTGGTATGTGGACGGGGTGGAGGTCCACAACGCCAAG
ACCAAACCCAGGGAGGAGCAGTACAACTCCACTTATCGCG
TGGTTTCTGTGCTGACAGTCCTGCACCAGGATTGGCTGAA
CGGGAAGGAGTACAAATGCAAAGTGTCCAATAAGGCCCTG
CCCGCCCCAATCGAGAAAACTATTTCAAAGGCCAAAGGAC
AGCCCAGAGAGCCACAGGTGACCTACCTCCCTCCTTCCAG
GGACGAGCTCACTAAGAATCAGGTGTCTCTGACTTGCCTG
GTGAAAGGCTTTTATCCTTCTGACATCGCAGTGGAGTGGG
AGAGCAATGGCCAGCCCGAGAACAATTATAAAACAACACC
ACCCGTCCTGGACTCTGATGGCAGCTTTTTCCTGTATAGC
AAGCTGACAGTGGACAAATCACGCTGGCAGCAGGGGAATG
TCTTCAGCTGTAGCGTGATGCACGAAGCTCTGCACAATCA
CTATACACAGAAGTCCCTGTCCCTGAGCCCAGGAAAATAA;
or
(D)
(SEQ ID NO: 5)
ATGGCTACCGGCAGCAGGACTAGCCTGCTGCTGGCTTTCG
GCCTGCTGTGTCTGCCTTGGCTGCAAGAGGGGTCCGCTTT
CCCTACTATCCCTCTGTCCGAAGTGCAGCTGGTCGAGAGC
GGAGGGGGCCTGGTGCAGCCTGGAAGAAGTCTGCGCCTGT
CCTGCGCAGCAAGCAGGTTTACATTTGACGACTACGCAAT
GCACTGGGTGCGCCAAGCTCCAGGCAAAGGCTTAGAATGG
GTGTCTGGCATCAGCTGGAACTCAGGGCGGATCGGCTACG
CAGACAGCGTGAAGGGCAGGTTCACTATCTCTAGGGACAA
CGCCGAGAACTCCCTGTTCCTGCAGATGAATGGGCTGCGG
GCAGAAGACACTGCACTGTATTATTGTGCTAAGGGGAGAG
ACTCTTTCGACATCTGGGGCCAGGGCACAATGGTGACTGT
GTCCTCTGCCTCTACCAAGGGCCCTTCCGTGTTCCCACTG
GCACCAAGCAGCAAATCCACATCCGGGGGGACCGCAGCTC
TCGGATGTCTGGTGAAAGACTATTTCCCTGAGCCCGTCAC
AGTGTCTTGGAATTCCGGCGCCCTGACAAGCGGCGTGCAC
ACTTTTCCTGCCGTTCTGCAGAGCTCCGGCCTATACTCCC
TGTCCAGCGTGGTGACAGTCCCTTCTAGCAGTCTGGGCAC
ACAGACTTATATTTGCAACGTGAATCACAAGCCATCTAAC
ACCAAGGTGGATAAGAAGGTGGAACCAAAGTCCTGTGATA
AAACCCATACCTGTCCTCCATGTCCAGCTCCTGAACTCCT
GGGGGGACCCTCTGTGTTCCTGTTCCCACCTAAGCCTAAA
GACACTCTGATGATTTCCAGAACTCCTGAGGTGACTTGCG
TGGTGGTGGATGTGTCCCATGAGGATCCTGAGGTCAAGTT
CAACTGGTACGTGGACGGAGTCGAGGTGCATAACGCTAAA
ACTAAACCAAGAGAGGAACAGTATAATTCCACTTATAGAG
TTGTGAGCGTCCTGACCGTGCTGCACCAGGACTGGCTGAA
CGGAAAAGAATACAAGTGTAAGGTGTCCAACAAGGCACTC
CCCGCACCAATTGAGAAGACCATTTCCAAGGCCAAGGGCC
AGCCAAGAGAGCCTCAGGTGACCTATCTGCCTCCAAGCCG
GGACGAACTGACAAAGAATCAGGTCAGCCTGACTTGCCTG
GTGAAGGGGTTTTACCCTTCTGACATCGCCGTGGAATGGG
AGTCTAATGGACAGCCCGAAAACAACTACAAGACCACACC
ACCCGTGCTGGACAGCGATGGCTCCTTTTTCCTGTATAGT
AAACTGACCGTCGACAAGTCTCGCTGGCAGCAGGGCAACG
TGTTTTCTTGCAGCGTTATGCATGAAGCCCTCCACAACCA
CTATACACAGAAAAGCCTGTCTCTCAGCCCTGGGAAGTGA.

In some embodiments, the mRNA that encodes the anti-IL6R antibody heavy chain is codon optimized and comprises a sequence 70%, 75%, 80%, 85%, 90%, 95% or more identical to one SEQ ID NO: 2, 3, 4, or 5. In some embodiments, the mRNA that encodes the anti-IL6R antibody heavy chain is codon optimized and comprises a sequence identical to one SEQ ID NO: 2, 3, 4, or 5. Accordingly, in some embodiments, the mRNA that encodes the anti-IL6R antibody heavy chain is codon optimized and comprises a sequence identical to SEQ ID NO: 2. In some embodiments, the mRNA that encodes the anti-IL6R antibody heavy chain is codon optimized and comprises a sequence identical to SEQ ID NO: 3. In some embodiments, the mRNA that encodes the anti-IL6R antibody heavy chain is codon optimized and comprises a sequence identical to SEQ ID NO: 4. In some embodiments, the mRNA that encodes the anti-IL6R antibody heavy chain is codon optimized and comprises a sequence identical to SEQ ID NO: 5.

In some embodiments, the mRNA that encodes the anti-IL6R antibody light chain is codon optimized and comprises a sequence at least 80% identical to one of the following sequences:

(A)
(SEQ ID NO: 6)
ATGGCCACTGGAAGCCGGACAAGCCTGCTGCTGGCCTTTG
GCCTGCTGTGTCTGCCTTGGCTGCAGGAGGGAAGCGCATT
TCCAACAATTCCTCTGAGCGACATTCAGATGACACAGAGC
CCCAGCAGCGTGTCCGCATCAGTGGGAGACAGGGTGACTA
TCACATGTAGAGCTTCTCAAGGAATTAGCTCTTGGCTGGC
CTGGTATCAGCAGAAGCCAGGCAAGGCCCCCAAGCTGCTG
ATCTATGGAGCTAGCTCTCTGGAGTCTGGGGTGCCATCTA
GGTTCAGTGGCTCCGGCAGCGGAACAGACTTCACACTGAC
TATCAGCAGCCTGCAGCCTGAGGACTTTGCCAGCTACTAC
TGCCAGCAGGCAAATAGCTTTCCCTATACTTTCGGACAGG
GCACCAAGCTGGAGATTAAGCGGACCGTTGCTGCTCCAAG
CGTGTTCATCTTCCCACCTTCCGACGAGCAGCTGAAGTCT
GGCACCGCCAGCGTGGTGTGTCTGCTGAACAATTTCTATC
CCCGTGAAGCCAAAGTGCAGTGGAAGGTGGATAACGCTCT
CCAGTCTGGCAATTCCCAGGAGAGCGTGACAGAGCAGGAT
TCTAAGGATTCTACCTACTCCCTGTCCAGCACACTGACCC
TGAGCAAGGCCGATTACGAAAAACACAAAGTGTACGCCTG
CGAAGTCACACACCAGGGGCTGAGCTCCCCAGTGACAAAG
AGCTTTAATAGAGGGGAGTGCTGA;
(B)
(SEQ ID NO: 7)
ATGGCTACAGGGAGCCGCACTAGCCTGCTGCTGGCTTTTG
GCCTGCTGTGCCTGCCATGGCTGCAAGAGGGGTCCGCCTT
TCCTACCATCCCCCTGTCCGATATTCAGATGACCCAGTCC
CCTAGCAGCGTGTCTGCCAGCGTGGGAGACAGGGTGACTA
TCACCTGTAGGGCCAGCCAGGGCATTTCTAGCTGGCTGGC
TTGGTACCAGCAGAAGCCAGGAAAGGCTCCCAAACTGCTG
ATCTACGGGGCATCCTCTCTGGAGTCCGGAGTGCCAAGCA
GATTCTCTGGGAGCGGCAGCGGGACCGATTTCACACTGAC
CATTAGCAGCCTGCAGCCAGAAGACTTCGCCAGCTACTAT
TGTCAGCAGGCAAACTCTTTTCCTTATACCTTCGGGCAGG
GGACTAAACTGGAAATCAAGCGGACAGTGGCTGCTCCAAG
CGTGTTCATCTTCCCACCTTCCGACGAGCAGCTGAAGTCT
GGCACAGCTTCCGTGGTGTGCCTGCTGAACAACTTTTATC
CAAGGGAAGCTAAAGTGCAGTGGAAGGTGGACAACGCTCT
GCAGTCTGGAAATAGCCAGGAATCCGTGACTGAGCAGGAT
AGCAAAGACAGCACATACAGCCTGTCTTCCACTCTGACCC
TGAGCAAGGCAGACTACGAGAAACACAAAGTGTATGCCTG
TGAGGTGACCCATCAGGGCCTGTCTAGCCCAGTGACCAAG
TCCTTTAACAGAGGCGAATGTTGA;
(C)
(SEQ ID NO: 8)
ATGGCAACTGGATCCCGGACCTCTCTGCTGCTGGCCTTCG
GACTGCTGTGCCTGCCATGGCTGCAGGAGGGGAGCGCTTT
TCCTACTATCCCCCTGTCTGACATCCAGATGACTCAGAGC
CCAAGCTCTGTGTCCGCAAGCGTGGGGGACAGGGTGACAA
TCACTTGCAGGGCATCCCAGGGAATCTCCTCTTGGCTGGC
ATGGTACCAGCAGAAACCTGGAAAAGCCCCAAAACTGCTG
ATTTATGGCGCTTCCAGCCTCGAATCCGGAGTGCCATCCC
GGTTTTCTGGCTCCGGCAGCGGGACAGATTTTACTCTGAC
CATCTCTAGCCTGCAGCCAGAGGATTTTGCCTCCTATTAT
TGCCAGCAGGCCAACAGCTTTCCTTATACCTTTGGACAGG
GAACTAAGCTGGAGATCAAGAGGACAGTGGCTGCTCCTAG
CGTGTTCATCTTCCCACCTTCTGACGAACAGCTGAAGTCT
GGAACAGCCTCTGTGGTGTGCCTCCTCAACAACTTCTATC
CCCGGGAGGCTAAGGTCCAGTGGAAAGTGGACAACGCCCT
GCAGTCCGGAAACTCCCAGGAGAGCGTGACCGAGCAGGAC
AGCAAGGATAGCACTTATTCCCTGTCCTCCACCCTGACTC
TGTCCAAGGCCGACTACGAAAAGCACAAAGTGTACGCCTG
CGAAGTCACTCATCAGGGACTGAGCTCCCCCGTGACCAAG
AGCTTCAATAGGGGAGAATGTTAG;
or
(D)
(SEQ ID NO: 9)
ATGGCAACTGGCTCCAGGACTAGCCTGCTGCTGGCATTTG
GCCTCCTGTGTCTGCCATGGCTGCAGGAGGGCTCCGCCTT
CCCAACAATTCCACTGTCCGACATCCAGATGACACAGTCC
CCTAGCAGCGTGAGCGCCTCCGTGGGAGATAGAGTGACAA
TTACCTGTCGCGCAAGCCAGGGGATCAGCAGCTGGCTGGC
CTGGTATCAACAGAAACCTGGAAAAGCCCCCAAGCTCCTG
ATCTATGGCGCCAGCAGCCTGGAAAGCGGGGTTCCAAGCC
GGTTTTCCGGGTCCGGCAGCGGAACTGACTTCACCCTGAC
AATTTCAAGCCTGCAGCCCGAGGATTTTGCAAGCTACTAC
TGTCAGCAGGCTAATAGCTTTCCTTACACATTCGGCCAGG
GCACCAAGCTCGAAATTAAAAGAACTGTGGCTGCCCCATC
CGTGTTTATCTTCCCACCCTCTGACGAACAGCTGAAGTCC
GGGACAGCCTCTGTGGTGTGCCTGCTGAACAATTTTTACC
CCAGGGAGGCTAAGGTCCAATGGAAGGTCGACAATGCTCT
GCAGTCTGGAAACTCCCAGGAGTCTGTGACTGAGCAGGAC
AGCAAGGACAGCACCTATAGCCTGTCTTCCACCCTGACCC
TGAGCAAGGCCGATTACGAAAAGCACAAGGTGTATGCCTG
TGAGGTGACCCACCAGGGACTGTCTAGCCCAGTGACTAAA
TCCTTTAATAGAGGCGAATGCTGA.

In some embodiments, the mRNA that encodes the anti-IL6R antibody light chain is codon optimized and comprises a sequence 70%, 75%, 80%, 85%, 90%, 95% or more identical to one of the following SEQ ID NO: 6, 7, 8, or 9. In some embodiments, the mRNA that encodes the anti-IL6R antibody light chain is codon optimized and comprises a sequence identical to one of the following SEQ ID NO: 6, 7, 8, or 9. In some embodiments, the mRNA that encodes the anti-IL6R antibody light chain is codon optimized and comprises a sequence identical to SEQ ID NO: 6. In some embodiments, the mRNA that encodes the anti-IL6R antibody light chain is codon optimized and comprises a sequence identical to SEQ ID NO: 7. In some embodiments, the mRNA that encodes the anti-IL6R antibody light chain is codon optimized and comprises a sequence identical to SEQ ID NO: 8. In some embodiments, the mRNA that encodes the anti-IL6R antibody light chain is codon optimized and comprises a sequence identical to SEQ ID NO: 9.

In some embodiments, the mRNA encodes an anti-IL4Rα antibody heavy chain comprising a sequence at least 80% identical to:

(SEQ ID NO: 20)
EVQLVESGGGLEQPGGSLRLSCAGSGFTFRDYAMTWVRQA
PGKGLEWVSSISGSGGNTYYADSVKGRFTISRDNSKNTLY
LQMNSLRAEDTAVYYCAKDRLSITIRPRYYGLDVWGQGTT
VTVSSASTKGPSVFPLAPCSRSTSESTAALGCLVKDYFPE
PVTVSWNSGALTSGVHTFPAVLQSSGLYSLSSVVTVPSSS
LGTKTYTCNVDHKPSNTKVDKRVESKYGPPCPPCPAPEFL
GGPSVFLFPPKPKDTLMISRTPEVTCVVVDVSQEDPEVQF
NWYVDGVEVHNAKTKPREEQFNSTYRVVSVLTVLHQDWLN
GKEYKCKVSNKGLPSSIEKTISKAKGQPREPQVYTLPPSQ
EEMTKNQVSLTCLVKGFYPSDIAVEWESNGQPENNYKTTP
PVLDSDGSFFLYSRLTVDKSRWQEGNVFSCSVMHEALHNH
YTQKSLSLSLG.

In some embodiments, the mRNA encodes an anti-IL4Rα antibody heavy chain comprising a sequence 70%, 75%, 80%, 85%, 90%, 95% or more identical to SEQ ID NO: 20. In some embodiments, the mRNA encodes an anti-IL4Rα antibody heavy chain comprising a sequence identical to SEQ ID NO: 20.

In some embodiments, the mRNA encodes an anti-IL4Rα antibody heavy chain further comprising a secretion sequence at least 80% identical to: MATGSRTSLLLAFGLLCLPWLQEGSAFPTIPLS (SEQ ID NO: 26). In some embodiments, the mRNA encodes an anti-IL4Rα antibody heavy chain further comprising a secretion sequence 70%, 80%, 85%, 90%, 95% or more identical to SEQ ID NO: 26. In some embodiments, the mRNA encodes an anti-IL4Rα antibody heavy chain further comprising a secretion sequence identical to SEQ ID NO: 26.

In some embodiments, the mRNA encodes an anti-IL4Rα antibody light chain comprising a sequence at least 80% identical to:

DIVMTQSPLSLPVTPGEPASISCRS SQSLLYSIGYNYLDWYLQKSGQSPQLLIYLGSNRAS GVPDRFSGSGSGTDFTLKISRVEAEDVGFYYCMQALQTPYTFGQGTKLEIKRTVAAPSV FIFPPSDEQLKSGTASVVCLLNNFYPREAKVQWKVDNALQSGNSQESVTEQDSKDSTYS LSSTLTLSKADYEKHKVYACEVTHQGLSSPVTKSFNRGEC (SEQ ID NO: 21). In some embodiments, the mRNA encodes an anti-IL4Rα antibody light chain comprising a sequence at 70%, 75%, 80%, 85%, 90%, 95% or more identical to SEQ ID NO: 21. In some embodiments, the mRNA encodes an anti-IL4Rα antibody light chain comprising a sequence identical to SEQ ID NO: 21.

In some embodiments, the mRNA encodes an anti-IL4Rα antibody light chain further comprising a secretion sequence at least 80% identical to: MATGSRTSLLLAFGLLCLPWLQEGSAFPTIPLS (SEQ ID NO: 26). In some embodiments, the mRNA encodes an anti-IL4Rα antibody light chain further comprising a secretion sequence 70%, 75%, 80%, 85%, 90%, 95% or more identical to SEQ ID NO: 26.

In some embodiments, the mRNA that encodes the anti-IL4Rα antibody heavy chain is codon optimized and comprises a sequence at least 80% identical to one of the following sequences:

(A)
(SEQ ID NO: 10)
ATGGCCACAGGCTCTAGGACATCCCTGCTGCTGGCCTTCG
GACTGCTGTGTCTGCCTTGGCTGCAGGAGGGATCTGCATT
CCCAACTATCCCTCTGTCCGAGGTTCAGCTGGTGGAAAGC
GGGGGAGGGCTGGAGCAGCCTGGGGGGTCCCTGAGACTGT
CCTGCGCTGGATCCGGCTTCACTTTTCGCGATTATGCCAT
GACATGGGTGCGGCAGGCCCCCGGCAAAGGACTGGAGTGG
GTTTCCAGCATTTCTGGCAGCGGAGGGAACACCTACTATG
CCGATAGCGTGAAGGGAAGGTTTACAATCAGCCGCGATAA
CAGCAAGAATACCCTCTATCTGCAGATGAATTCTCTGAGG
GCAGAGGACACTGCCGTGTATTATTGCGCAAAGGATAGGC
TGAGCATCACTATCCGCCCACGCTACTACGGGCTGGACGT
GTGGGGGCAGGGAACTACCGTTACCGTGTCTTCCGCCAGC
ACAAAGGGACCTTCTGTGTTCCCCCTGGCTCCCTGTAGCA
GATCCACCTCTGAGAGCACCGCTGCCCTGGGATGCCTGGT
GAAGGATTATTTCCCAGAGCCCGTGACTGTGAGCTGGAAT
TCAGGCGCACTCACCTCTGGGGTGCACACCTTCCCTGCCG
TGCTGCAGTCCAGCGGCCTGTATTCTCTCTCCAGCGTCGT
GACCGTGCCTTCCAGTAGCCTGGGAACTAAAACATATACC
TGTAACGTGGATCACAAGCCCTCCAATACCAAGGTGGACA
AGCGGGTCGAGAGCAAGTACGGACCCCCATGTCCTCCCTG
TCCAGCTCCTGAGTTCCTGGGGGGCCCTTCAGTGTTCCTG
TTTCCCCCTAAGCCAAAGGACACTCTCATGATCTCCAGGA
CTCCAGAGGTGACATGCGTGGTGGTGGATGTCAGCCAGGA
GGATCCAGAGGTCCAGTTCAATTGGTACGTCGACGGGGTG
GAGGTGCATAACGCCAAGACTAAGCCCCGCGAGGAACAGT
TTAATTCCACTTACAGGGTGGTCTCTGTGCTGACTGTCCT
GCATCAGGATTGGCTGAACGGAAAGGAGTATAAGTGCAAA
GTGTCTAATAAGGGGCTGCCCAGCTCCATCGAGAAAACAA
TCTCTAAGGCTAAGGGGCAGCCTCGGGAGCCTCAGGTGTA
CACTCTGCCCCCTTCACAGGAAGAGATGACCAAAAATCAG
GTGTCCCTGACTTGCCTGGTGAAGGGGTTTTATCCCTCTG
ACATCGCAGTGGAATGGGAGTCCAACGGCCAGCCTGAAAA
CAACTATAAGACAACCCCTCCCGTGCTGGATAGCGACGGG
AGCTTTTTCCTGTACAGCAGACTGACTGTGGATAAATCTA
GGTGGCAGGAGGGAAACGTGTTTTCTTGCAGCGTCATGCA
CGAAGCCCTGCACAATCACTACACACAGAAATCCCTGTCC
CTGTCCCTGGGCTGA;
(B)
(SEQ ID NO: 11)
ATGGCTACCGGGTCCAGGACATCTCTGCTGCTGGCCTTCG
GACTGCTGTGCCTGCCATGGCTGCAGGAAGGCTCAGCCTT
TCCAACAATCCCACTGTCCGAAGTGCAGCTGGTGGAGAGC
GGCGGCGGCCTTGAACAACCTGGAGGCTCTTTGAGACTGT
CATGCGCCGGGTCCGGATTTACCTTTCGCGACTACGCAAT
GACTTGGGTGCGCCAGGCTCCCGGAAAGGGACTGGAATGG
GTTTCCTCTATTAGCGGGTCCGGCGGCAACACTTATTACG
CAGATAGCGTGAAGGGGCGCTTCACTATTAGCAGGGACAA
TTCTAAAAACACCCTGTACCTGCAGATGAACAGCTTAAGA
GCCGAAGACACAGCTGTGTACTACTGCGCTAAAGACAGAC
TCTCCATTACAATCCGCCCAAGGTATTACGGCCTGGACGT
GTGGGGCCAGGGAACAACAGTGACCGTGAGCTCTGCTTCC
ACTAAGGGCCCTAGCGTGTTCCCCCTGGCTCCATGCTCCC
GCAGCACATCAGAGTCTACCGCCGCACTGGGATGTCTGGT
GAAGGATTACTTCCCCGAGCCTGTGACTGTGAGCTGGAAT
AGCGGGGCCCTGACCTCTGGAGTTCATACATTCCCAGCCG
TGCTGCAGTCTTCCGGCCTGTACTCTCTGAGCTCTGTGGT
GACCGTCCCATCCTCTTCTCTGGGCACAAAGACCTACACA
TGTAACGTTGACCACAAGCCATCCAATACCAAGGTGGACA
AGAGAGTGGAATCCAAGTATGGCCCTCCTTGTCCCCCTTG
TCCTGCTCCAGAGTTCCTGGGAGGGCCATCCGTCTTCCTC
TTCCCTCCCAAGCCTAAGGATACACTGATGATCTCCAGGA
CCCCTGAAGTGACATGTGTCGTGGTGGACGTGAGCCAAGA
AGACCCCGAGGTGCAGTTCAACTGGTACGTGGACGGAGTC
GAGGTGCACAACGCTAAAACAAAGCCCCGCGAGGAGCAGT
TCAACTCCACATACCGGGTGGTCTCAGTGCTGACTGTGCT
TCATCAGGATTGGCTGAATGGGAAGGAGTACAAGTGCAAG
GTGAGCAACAAGGGACTGCCATCTAGCATCGAGAAAACAA
TCAGCAAGGCTAAGGGACAGCCAAGGGAACCTCAGGTGTA
TACTCTGCCACCCTCCCAGGAAGAGATGACTAAGAATCAG
GTCTCCCTGACCTGTCTGGTGAAGGGATTCTACCCTAGCG
ACATTGCTGTCGAGTGGGAGTCCAACGGGCAGCCAGAAAA
TAATTACAAGACCACACCTCCAGTGCTGGACAGCGATGGA
TCCTTCTTCCTGTACTCTCGGCTGACCGTGGATAAGAGCC
GGTGGCAGGAGGGCAACGTTTTCTCTTGCAGCGTGATGCA
CGAGGCTCTGCATAATCACTATACACAGAAGTCTCTAAGC
CTGTCTCTGGGATGA;
(C)
(SEQ ID NO: 12)
ATGGCTACAGGATCCCGGACTAGCCTGCTGCTGGCCTTCG
GCCTGTTGTGCCTGCCTTGGCTGCAGGAGGGGTCTGCCTT
TCCAACAATCCCACTGTCTGAGGTCCAGCTGGTGGAGTCC
GGCGGAGGGCTAGAACAGCCTGGGGGATCTCTGAGGCTCT
CTTGCGCAGGATCCGGCTTTACATTCAGAGACTACGCAAT
GACTTGGGTCAGACAGGCCCCTGGAAAGGGGCTGGAGTGG
GTTTCCAGCATTTCCGGATCCGGGGGCAACACATATTACG
CTGACTCTGTGAAGGGCAGGTTCACAATCAGCAGGGATAA
CTCCAAGAACACCCTCTATCTGCAGATGAACTCCCTGCGG
GCCGAGGATACCGCAGTGTACTACTGTGCCAAAGATAGGC
TGAGCATCACAATCCGCCCTAGGTATTATGGGCTCGACGT
GTGGGGCCAGGGAACTACAGTGACAGTGTCCTCGGCATCC
ACCAAAGGCCCCTCCGTTTTCCCCCTGGCACCCTGTAGCC
GCTCTACTTCTGAGAGTACTGCTGCCCTGGGCTGCCTGGT
GAAGGATTACTTTCCAGAGCCCGTCACAGTGTCCTGGAAT
TCTGGGGCTCTGACTTCTGGCGTGCACACATTCCCCGCAG
TGCTGCAGTCTTCTGGCCTGTACTCTCTGTCTTCTGTGGT
GACCGTGCCCAGCAGCAGCCTGGGCACTAAGACATATACC
TGTAATGTTGACCACAAACCTTCCAACACTAAGGTGGACA
AGAGGGTGGAGTCTAAGTATGGACCTCCCTGTCCACCTTG
TCCTGCTCCAGAGTTCCTCGGGGGACCAAGCGTTTTCCTG
TTCCCCCCAAAGCCAAAGGACACTCTGATGATTAGCCGCA
CTCCCGAAGTGACTTGTGTTGTGGTGGACGTCTCTCAGGA
GGATCCTGAGGTGCAGTTTAATTGGTACGTGGATGGCGTG
GAGGTGCACAACGCCAAGACAAAACCACGGGAGGAACAGT
TCAATAGCACCTATAGGGTCGTGTCCGTCCTGACAGTGCT
CCACCAGGATTGGCTCAACGGAAAAGAATACAAATGCAAG
GTGTCTAACAAGGGGCTGCCTTCCAGCATCGAGAAGACTA
TTAGCAAGGCAAAGGGGCAGCCAAGAGAGCCTCAGGTGTA
TACCCTGCCCCCATCTCAGGAGGAGATGACAAAGAACCAG
GTCTCCCTGACTTGTCTGGTCAAGGGGTTCTACCCATCTG
ACATCGCTGTGGAGTGGGAGAGCAACGGCCAACCCGAGAA
TAACTACAAAACAACCCCACCCGTGCTGGACAGCGATGGA
TCCTTCTTCCTGTATTCCAGGTTGACCGTGGACAAATCTC
GCTGGCAGGAGGGAAACGTTTTCTCTTGCTCCGTGATGCA
CGAGGCCCTGCACAATCACTACACACAGAAATCTCTCTCT
CTGTCTCTGGGGTGA;
or
(D)
(SEQ ID NO: 13)
ATGGCTACAGGGTCTCGGACAAGTCTGCTGCTGGCATTCG
GGCTGCTGTGCCTGCCATGGCTGCAAGAGGGAAGCGCATT
CCCAACCATTCCACTCAGCGAGGTGCAGCTGGTCGAAAGC
GGGGGGGGACTGGAACAACCTGGAGGATCCCTGCGGCTGT
CATGCGCAGGCTCCGGCTTTACCTTCAGGGACTACGCCAT
GACATGGGTGAGACAGGCTCCTGGGAAGGGGCTCGAGTGG
GTGAGCAGCATTTCCGGAAGCGGGGGAAACACCTATTACG
CAGATAGTGTTAAGGGCCGCTTTACTATCTCTAGGGACAA
TTCCAAGAACACCCTGTACCTGCAGATGAACAGCCTGCGA
GCCGAGGACACCGCAGTGTATTACTGTGCCAAGGACCGGC
TCTCTATTACCATTAGACCTAGGTATTACGGGCTGGACGT
GTGGGGACAGGGAACAACAGTGACCGTGTCTTCTGCCTCC
ACAAAAGGGCCCTCTGTGTTCCCTCTGGCACCTTGCTCCA
GGTCTACCTCCGAGAGCACAGCTGCACTGGGATGTCTGGT
TAAAGATTACTTTCCAGAACCAGTTACTGTGAGCTGGAAC
TCTGGAGCTCTGACCTCCGGAGTTCACACATTCCCTGCAG
TGCTGCAGTCTAGCGGCCTGTATTCCCTGTCCTCCGTCGT
GACCGTGCCTTCCTCCTCTCTGGGCACTAAGACCTACACT
TGCAACGTGGATCACAAACCTAGCAATACAAAGGTCGATA
AACGGGTTGAGAGCAAATACGGCCCTCCATGTCCTCCTTG
TCCAGCCCCTGAATTCCTGGGCGGACCCTCCGTTTTCCTG
TTCCCACCCAAGCCCAAGGACACACTGATGATTTCTAGGA
CTCCTGAAGTGACATGCGTGGTCGTGGATGTCTCCCAGGA
GGATCCAGAAGTCCAGTTCAATTGGTACGTGGATGGAGTG
GAGGTGCACAATGCCAAGACAAAGCCAAGGGAGGAGCAGT
TTAACTCTACTTACAGAGTGGTGAGCGTGCTCACAGTGCT
GCATCAGGATTGGCTCAACGGAAAAGAGTACAAGTGTAAG
GTCAGCAATAAGGGCCTGCCATCCTCCATTGAGAAAACCA
TCTCCAAGGCAAAGGGGCAGCCAAGAGAACCTCAGGTCTA
CACCCTGCCACCATCTCAAGAGGAGATGACCAAGAATCAG
GTGAGCCTCACTTGCCTGGTGAAGGGATTCTACCCTAGCG
ACATTGCCGTGGAGTGGGAATCTAACGGGCAGCCAGAGAA
CAACTACAAGACAACTCCTCCCGTGCTGGATAGCGACGGG
TCTTTCTTCCTGTATAGCAGGCTGACAGTGGATAAGAGCC
GCTGGCAAGAGGGCAACGTCTTTTCTTGTTCCGTCATGCA
CGAGGCTCTGCATAACCACTATACCCAGAAGTCACTGTCC
CTCTCCCTGGGGTGA.

In some embodiments, the mRNA that encodes the anti-IL4Rα antibody heavy chain is codon optimized and comprises a sequence 70%, 75%, 80%, 85%, 90%, 95% or more identical to one of SEQ ID NO: 10, 11, 12, or 13. In some embodiments, the mRNA that encodes the anti-IL4Rα antibody heavy chain is codon optimized and comprises a sequence identical to one of SEQ ID NO: 10, 11, 12, or 13. Accordingly, in some embodiments, the mRNA that encodes the anti-IL4Rα antibody heavy chain is codon optimized and comprises a sequence identical to SEQ ID NO: 10. In some embodiments, the mRNA that encodes the anti-IL4Rα antibody heavy chain is codon optimized and comprises a sequence identical to SEQ ID NO: 11. In some embodiments, the mRNA that encodes the anti-IL4Rα antibody heavy chain is codon optimized and comprises a sequence identical to SEQ ID NO: 12. In some embodiments, the mRNA that encodes the anti-IL4Rα antibody heavy chain is codon optimized and comprises a sequence identical to SEQ ID NO: 13.

In some embodiments, the mRNA that encodes the anti-IL4Rα antibody light chain is codon optimized and comprises a sequence at least 80% identical to one of the following sequences:

(A)
(SEQ ID NO: 14)
ATGGCCACAGGCTCTAGGACATCCCTGCTGCTGGCCTTCG
GACTGCTGTGTCTGCCTTGGCTGCAGGAGGGATCTGCATT
CCCAACTATCCCTCTGTCCGATATTGTGATGACCCAGAGC
CCCCTGAGCCTGCCAGTGACTCCTGGGGAGCCCGCATCTA
TCAGCTGCCGGTCCTCTCAGTCTCTGCTGTATTCTATCGG
GTACAACTACCTGGATTGGTACCTGCAGAAAAGTGGGCAG
AGCCCCCAGCTGCTCATCTATCTGGGGTCCAACAGGGCTA
GTGGCGTGCCAGACCGGTTCTCCGGATCCGGCTCCGGAAC
AGACTTTACACTGAAAATTAGCCGCGTGGAGGCCGAGGAC
GTGGGGTTTTATTATTGTATGCAGGCCCTGCAGACCCCAT
ACACATTTGGCCAGGGGACAAAGCTGGAAATTAAGCGCAC
TGTGGCCGCTCCGTCTGTGTTCATCTTTCCTCCCAGCGAT
GAACAGCTGAAGTCTGGGACCGCTAGCGTCGTGTGCCTGC
TGAACAATTTTTACCCCAGGGAGGCTAAGGTGCAGTGGAA
GGTGGACAATGCCCTGCAGAGCGGGAACAGCCAGGAGAGT
GTTACTGAGCAGGATTCTAAAGATTCCACCTATTCCCTGT
CTTCCACCCTGACTCTGTCTAAGGCCGATTACGAAAAACA
TAAGGTGTACGCATGCGAGGTGACCCACCAGGGGCTGAGC
TCTCCCGTGACTAAGAGCTTCAATCGCGGAGAGTGCTGA;
(B)
(SEQ ID NO: 15)
ATGGCTACAGGCAGCAGAACCAGCCTGCTGCTGGCATTTG
GCCTGCTGTGCCTGCCTTGGCTGCAGGAGGGGAGCGCTTT
TCCCACAATTCCTCTGTCTGATATCGTCATGACCCAATCT
CCCCTGTCCCTGCCTGTGACTCCAGGAGAGCCCGCTAGCA
TTTCTTGCAGGTCTTCCCAGAGCCTGCTGTACAGCATCGG
CTATAACTACCTGGATTGGTATCTGCAGAAAAGCGGGCAG
TCTCCTCAGCTGCTGATCTACCTGGGCTCTAACAGAGCCT
CTGGGGTCCCCGACAGGTTTTCCGGAAGCGGCTCTGGCAC
CGACTTTACTCTCAAAATCAGCCGCGTGGAGGCAGAGGAC
GTGGGCTTCTATTACTGCATGCAGGCCCTGCAGACACCAT
ATACATTCGGACAGGGGACCAAGCTGGAGATTAAGAGAAC
AGTGGCTGCCCCAAGCGTGTTTATCTTTCCTCCCTCCGAT
GAACAGCTGAAAAGCGGCACTGCTTCCGTGGTGTGCCTGC
TGAATAATTTCTACCCTAGAGAGGCCAAAGTCCAGTGGAA
GGTGGACAACGCCCTGCAGAGCGGGAACAGCCAGGAAAGT
GTCACCGAGCAGGATTCCAAGGATTCCACATATTCTCTGT
CCAGCACTCTGACACTGTCCAAGGCAGACTACGAAAAACA
CAAGGTCTACGCCTGCGAAGTGACCCACCAGGGACTGTCT
AGCCCTGTGACTAAGTCTTTTAATAGGGGGGAGTGTTAG;
(C)
(SEQ ID NO: 16)
ATGGCTACTGGCAGCAGAACCAGCCTGCTGCTGGCATTCG
GGCTGCTCTGCCTGCCATGGCTGCAGGAGGGATCCGCCTT
CCCAACTATCCCCCTGAGCGATATCGTGATGACCCAGTCT
CCCCTGAGCCTGCCAGTTACACCCGGCGAACCTGCTAGCA
TCAGCTGCAGATCCTCCCAGTCTCTCCTGTACTCCATCGG
GTACAATTATCTGGATTGGTATCTGCAGAAGTCTGGCCAA
TCCCCCCAGCTGCTGATCTACCTGGGCTCCAACAGAGCAA
GCGGCGTGCCCGATAGATTCAGCGGCAGCGGGAGCGGCAC
TGATTTTACTCTGAAGATCAGCAGGGTGGAGGCCGAAGAT
GTGGGATTTTACTACTGCATGCAAGCACTGCAGACTCCTT
ACACATTCGGCCAGGGAACTAAGCTGGAGATCAAAAGAAC
CGTGGCAGCTCCAAGCGTCTTCATTTTCCCACCTTCTGAC
GAGCAGCTGAAGTCCGGCACAGCTTCCGTCGTGTGCCTCC
TGAACAACTTCTACCCCAGGGAGGCAAAGGTGCAATGGAA
AGTGGACAACGCTCTGCAGAGCGGAAACAGTCAGGAGTCC
GTGACCGAGCAGGACAGCAAAGACTCCACTTACAGCCTGA
GCTCTACTCTGACCCTGAGCAAAGCTGACTACGAGAAGCA
TAAGGTGTATGCTTGCGAGGTCACCCACCAGGGCCTCTCT
TCTCCCGTGACCAAGAGCTTCAACAGAGGCGAGTGCTGA;
or
(D)
(SEQ ID NO: 17)
ATGGCAACTGGAAGCAGGACCTCCCTGCTCCTGGCTTTCG
GCCTGCTCTGTCTGCCATGGCTGCAAGAAGGATCTGCCTT
TCCTACAATTCCACTGTCCGACATCGTGATGACACAGTCC
CCCCTGTCTCTGCCTGTCACCCCAGGCGAACCAGCCTCTA
TTTCTTGTCGGTCCTCTCAGTCCCTGCTGTATAGCATCGG
ATATAATTATCTGGACTGGTACCTGCAAAAATCCGGCCAG
TCTCCTCAGCTGCTGATCTATCTGGGCTCCAACCGGGCTA
GCGGAGTCCCAGACCGGTTTTCCGGGTCTGGCAGTGGGAC
AGATTTTACACTGAAAATTTCCCGGGTGGAGGCTGAGGAC
GTGGGATTTTACTACTGTATGCAGGCCCTGCAAACCCCAT
ATACTTTCGGACAGGGAACAAAGCTGGAGATCAAAAGAAC
CGTGGCCGCCCCCAGCGTTTTCATCTTCCCACCAAGCGAC
GAGCAGCTCAAATCTGGGACCGCTAGCGTGGTCTGTCTGC
TGAATAACTTCTACCCAAGGGAAGCAAAGGTGCAGTGGAA
GGTCGACAACGCACTGCAGAGCGGGAACTCCCAGGAGAGC
GTGACTGAACAGGACAGCAAGGACAGCACCTATAGCCTCA
GCAGCACTCTGACCCTGTCTAAAGCTGATTACGAAAAACA
CAAGGTGTATGCTTGTGAAGTGACTCACCAGGGCCTGTCT
TCCCCTGTTACAAAGTCCTTCAATAGAGGAGAATGTTAA.

In some embodiments, the mRNA that encodes the anti-IL4Rα antibody light chain is codon optimized and comprises a sequence 70%, 75%, 80%, 85%, 90%, 95% or more identical to SEQ ID NO: 14, 15, 16, or 17. In some embodiments, the mRNA that encodes the anti-IL4Rα antibody light chain is codon optimized and comprises a sequence identical to SEQ ID NO: 14, 15, 16, or 17. Accordingly, in some embodiments, the mRNA that encodes the anti-IL4Rα antibody light chain is codon optimized and comprises a sequence identical to SEQ ID NO: 14. In some embodiments, the mRNA that encodes the anti-IL4Rα antibody light chain is codon optimized and comprises a sequence identical to SEQ ID NO: 15. In some embodiments, the mRNA that encodes the anti-IL4Rα antibody light chain is codon optimized and comprises a sequence identical to SEQ ID NO: 16. In some embodiments, the mRNA that encodes the anti-IL4Rα antibody light chain is codon optimized and comprises a sequence identical to SEQ ID NO: 17.

In some embodiments, the mRNA comprises a 5′ UTR and a 3′UTR sequence. In some embodiments, the 5′ UTR sequence comprises a sequence with at least 80% identity to GGACAGATCGCCTGGAGACGCCATCCACGCTGTTTTGACCTCCATAGAAGACACCG GGACCGATCCAGCCTCCGCGGCCGGGAACGGTGCATTGGAACGCGGATTCCCCGTG CCAAGAGTGACTCACCGTCCTTGACACG (SEQ ID NO: 27). In some embodiments, the 5′ UTR sequence comprises a sequence 70%, 75%, 80%, 85%, 90%, 95% or more identity to SEQ ID NO: 27. In some embodiments, the 5′ UTR sequence comprises a sequence identical to SEQ ID NO: 27.

In some embodiments, the 3′ UTR sequence comprises a sequence with at least 80% identity to: CGGGTGGCATCCCTGTGACCCCTCCCCAGTGCCTCTCCTGGCCCTGGAAGTTGCCAC TCCAGTGCCCACCAGCCTTGTCCTAATAAAATTAAGTTGCATC (SEQ ID NO: 28). In some embodiments, the 3′ UTR sequence comprises a sequence 70%, 75%, 80%, 85%, 90%, 95% or more identical to SEQ ID NO: 28. In some embodiments, the 3′ UTR sequence comprises a sequence identical to SEQ ID NO: 28.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1 is an exemplary graph that compares human IgG levels in Bronchoalveolar Lavage Fluid (BALF) 72 h after administration of mRNA encoding anti-IL6R or anti-IL4Rα antibodies in mice relative to control mice administered a saline control.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Antibody: As used herein, the term “antibody” encompasses both intact antibodies and active antibody fragments. Typically, an intact “antibody” is an immunoglobulin that binds specifically to a particular antigen. An antibody may be a member of any immunoglobulin class, including any of the human classes: IgG, IgM, IgA, IgE, and IgD. A typical immunoglobulin (antibody) structural unit as understood in the art, is known to comprise a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these light and heavy chains respectively. Each variable region is further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). In some embodiments, the terms “intact antibody” or “fully assembled antibody” are used in reference to an antibody to mean that it contains two heavy chains and two light chains, optionally associated by disulfide bonds as occurs with naturally-produced antibodies. In some embodiments, an antibody according to the present invention is an antibody fragment. As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR) region.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or grammatical equivalent, refers to the process of confining an mRNA molecule within a nanoparticle.

Engineered or mutant: As used herein, the terms “engineered” or “mutant”, or grammatical equivalents refer to a nucleotide or protein sequence comprising one or more modifications compared to its naturally-occurring sequence, including but not limited to deletions, insertions of heterologous nucleic acids or amino acids, inversions, substitutions, or combinations thereof.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Impurities: As used herein, the term “impurities” refers to substances inside a confined amount of liquid, gas, or solid, which differ from the chemical composition of the target material or compound. Impurities are also referred to as contaminants.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

“Nebulization” refers to delivery of a pharmaceutical composition in a fine spray or dispersed suspension that is inhaled into the lungs, typically by means of a nebulizer.

“Nebulizer” is a device that uses a propellant or other suitable energy source such as oxygen, compressed air, or ultrasound waves to convert liquid or particles into a fine spray or mist or a dispersed suspension, typically in form of an aerosol that can be directly inhaled. In some embodiments, a nebulizer for use with the invention contains a piezoelectric element to generate the vibration of a mesh. The vibration pumps a liquid pharmaceutical composition through the mesh. The liquid is emitted from the mesh in droplets generating the aerosol. Such nebulizers are commonly referred to as (vibrating) mesh nebulizers. In some embodiments, a nebulizer is used to aerosolize a pharmaceutical composition for pulmonary delivery. Inhalation from a nebulizer is through a mouthpiece used by the subject.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. In some embodiments, nucleic acids are purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

“Pulmonary delivery” refers to administering the pharmaceutical composition described herein to lung cells in vivo by delivering the pharmaceutical composition to the lung. Non-limiting methods of pulmonary delivery include: nebulization and intratracheal administration/intratracheal instillation.

Stable: As used herein, the term “stable” protein or its grammatical equivalents refer to protein that retains its physical stability and/or biological activity. In one embodiment, protein stability is determined based on the percentage of monomer protein in the solution, at a low percentage of degraded (e.g., fragmented) and/or aggregated protein. In one embodiment, a stable engineered protein retains or exhibits an enhanced half-life as compared to a wild-type protein. In one embodiment, a stable engineered protein is less prone to ubiquitination that leads to proteolysis as compared to a wild-type protein.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, improved methods and compositions for the delivery of mRNA encapsulated within a lipid nanoparticle, wherein the mRNA encodes an antibody that targets drivers of Type II inflammation. For example, suitable protein targets associated with Type II inflammation include IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1). For example, the antibodies of the present invention bind and/or inhibit IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1). Various aspects of the invention are further described below.

mRNA Coded Antibodies.

The present disclosure provides mRNA-coded antibodies that can be used for the treatment of various disease, including for example, immune-related diseases. Various immune-related disease are known in the art and can be generally divided into immune-related disease of the lung and immune-related disease not associated with the lung. The methods and compositions of mRNA coded antibodies provided herewith can be used in the treatment of either lung-related or non-lung related immune disease. Examples of lung-related immune disease include, for example, asthma, chronic rhinosinusitis with nasal polyps (CRSwNP), chronic obstructive pulmonary disease (COPD), systemic sclerodis—interstitial lung disease (SSc-ILD), idiopathic pulmonary fibrosis IPF, sarcoidosis, or allergy. Examples of non-lung related diseases include autoimmune hepatitis and atopic dermatitis.

Methods are provided for treating an immune disease in a subject, comprising administering to a subject in need thereof one or more mRNAs encoding a heavy chain and a light chain of an antibody that binds and/or inhibits a protein target selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP).

Methods are provided for treating an immune disease in a subject, comprising administering to a subject in need thereof one or more mRNAs encoding a heavy chain and a light chain of an antibody that binds a protein target selected IL-4, IL-5, IL-6, IL-9, IL-13, IL-25, or IL-33, and wherein the one or more mRNAs are encapsulated in a lipid nanoparticle (LNP).

Methods are provided for treating an immune disease in a subject, comprising administering to a subject in need thereof one or more mRNAs encoding a heavy chain and a light chain of an antibody that binds a protein target selected from IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP).

Methods are provided for treating an immune disease in a subject, comprising administering to a subject in need thereof one or more mRNAs encoding a heavy chain and a light chain of an antibody that inhibits a protein target selected IL-4, IL-5, IL-6, IL-9, IL-13, IL-25, or IL-33, and wherein the one or more mRNAs are encapsulated in a lipid nanoparticle (LNP).

Methods are provided for treating an immune disease in a subject, comprising administering to a subject in need thereof one or more mRNAs encoding a heavy chain and a light chain of an antibody that inhibits a protein target selected from IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP).

In some aspects, the mRNA coded antibodies encapsulated in an LNP are used to treat lung-related immune diseases. In this scenario, the mRNA encoding an antibody and encapsulated within an LNP is delivered to the lung tissue by inhalation, nebulization or by intratracheal instillation. One advantage with this administration method is that for lung related diseases it allows for low to no systemic exposure and thus avoids unwanted systemic side-effects. Accordingly, in some embodiments, administering the mRNA encoding an antibody by inhalation or nebulization does not result in systemic exposure. In some embodiments, administering the mRNA encoding an antibody by inhalation or nebulization has low systemic exposure. In some embodiments, administering the mRNA encoding an antibody by inhalation or nebulization results in delivery of the mRNA encapsulated in the LNP to lung tissue.

In some embodiments, the mRNA coded antibody is an anti-IL6R antibody. Various anti-IL6R antibodies are known in the art and include those such as sarilumab and tocilizumab. Anti-IL6R antibodies have been used in the treatment of various diseases including rheumatoid arthritis, polyarticular juvenile idiopathic arthritis, systemic juvenile arthritis, and to treat cytokine storm in cases of severe coronavirus infection or Covid-19 disease.

In some embodiments, the mRNA encoded antibodies provided herein are used in the treatment of rheumatoid arthritis. In some embodiments, the anti-IL6R mRNA encoded antibodies provided herein are used in the treatment of rheumatoid arthritis. In some embodiments, the anti-IL6R mRNA encoded antibodies provided herein are used in the treatment of polyarticular juvenile idiopathic arthritis. In some embodiments, the anti-IL6R mRNA encoded antibodies provided herein are used in the treatment of systemic juvenile arthritis. In these scenarios, the anti-IL6R mRNA coded antibodies are delivered intravenously or intraperitoneally.

In some embodiments, the mRNA encoded antibodies provided herein are used to reduce or treat cytokine storm in a subject who has coronavirus infection or Covid-19 disease. In some embodiments, the mRNA encoded antibodies provided herein can be used to treat a subject who has a coronavirus infection or Covid-19 disease. In this scenario the mRNA coded antibodies are delivered by inhalation or nebulization.

In some embodiments, the mRNA coded antibody is an anti-IL4a antibody. Various anti-IL4a antibodies are known in the art and include, for example dupilumab. Anti-IL4a antibodies have been used in the treatment of various disease including, for example, treatment of atopic dermatitis, asthma with eosinophilic phenotype or with oral corticosteroid-dependent asthma, chronic rhinosinusitis with nasal polyposis, and is being evaluated for diseases driven by type 2 inflammation such as pediatric atopic dermatitis, pediatric asthma, eosphinohilic esophagitis, COPD, prurigo nodularis, chronic spontaneous urticaria and bullous phemphigoid. Anti-IL4a antibodies have also been used in the treatment of grass pollen allergy and peanut allergy.

In some embodiments, the mRNA coded antibodies provided herein can be used to treat one or more of atopic dermatitis, asthma with eosinophilic phenotype or with oral corticosteroid-dependent asthma, chronic rhinosinusitis with nasal polyposis, pediatric atopic dermatitis, pediatric asthma, eosphinohilic esophagitis, COPD, prurigo nodularis, chronic spontaneous urticaria and bullous phemphigoid, grass pollen allergy and peanut allergy.

Accordingly, in some embodiments, the anti-IL4a mRNA coded antibodies provided herein are used to treat atopic dermatitis. In some embodiments, the anti-IL4a mRNA coded antibodies provided herein are used to treat moderate atopic dermatitis. In some embodiments, the anti-IL4a mRNA coded antibodies provided herein are used to treat severe atopic dermatitis. In some embodiments, the anti-IL4a mRNA coded antibodies provided herein are used to treat asthma. In some embodiments, the anti-IL4a mRNA coded antibodies provided herein are used to treat severe asthma. In some embodiments, the anti-IL4a mRNA coded antibodies provided herein are used to treat moderate asthma. In some embodiments, the anti-IL4a mRNA coded antibodies provided herein are used to treat chronic rhinosinusitis with nasal polyposis. In some embodiments, the anti-IL4a mRNA coded antibodies provided herein are used to treat prurigo nodularis. In some embodiments, the anti-IL4a mRNA coded antibodies provided herein are used to treat eosinophilic esophagitis. In some embodiments, the anti-IL4a mRNA coded antibodies provided herein are used to treat bullous pemphigoid. In some embodiments, the anti-IL4a mRNA coded antibodies provided herein are used to treat chronic spontaneous urticaria. In some embodiments, the anti-IL4a mRNA coded antibodies provided herein are used to treat COPD. In some embodiments, the anti-IL4a mRNA coded antibodies provided herein are used to treat grass pollen allergy. In some embodiments, the anti-IL4a mRNA coded antibodies provided herein are used to treat peanut allergy.

Exemplary sequences of mRNA that encode the antibodies described herein is provided in the Table below. In some embodiments, the nucleotide sequences are codon optimized sequences.

TABLE 1
Codon-optimized Anti-IL6 and Anti-IL4 heavy
and light chain nucleotide sequences

Anti-IL6 Heavy Chain SEQ ID NO: 2
ATGGCCACTGGAAGCCGGACAAGCCTGCTGCTGGCCTTTGGCCTGCTGTGTCTGCCTTGGCTG
CAGGAGGGAAGCGCATTTCCAACAATTCCTCTGAGCGAAGTGCAGCTGGTGGAGTCTGGAGGA
GGCCTCGTGCAGCCAGGCAGATCCCTGAGGCTCTCCTGCGCCGCTAGCAGATTCACTTTCGAC
GACTACGCCATGCACTGGGTGCGGCAGGCACCTGGCAAGGGACTGGAATGGGTGTCCGGCATT
TCTTGGAACAGCGGGCGGATCGGGTACGCGGACAGCGTGAAAGGAAGGTTTACAATCTCCCGG
GACAATGCTGAGAACTCCCTGTTCCTGCAGATGAACGGCCTGAGAGCTGAAGACACAGCACTG
TACTATTGCGCAAAAGGCCGCGACTCCTTTGACATCTGGGGGCAGGGCACAATGGTGACCGTG
TCTAGCGCCTCCACAAAAGGACCTAGCGTTTTCCCACTGGCTCCATCTAGCAAGTCTACATCC
GGGGGCACCGCCGCTCTGGGCTGTCTGGTGAAGGATTACTTCCCTGAGCCCGTCACTGTCAGC
TGGAACTCCGGAGCTCTGACCTCAGGCGTGCACACTTTTCCCGCTGTGCTGCAGAGCTCTGGC
CTGTACAGCCTGAGCAGCGTTGTGACCGTGCCTAGCTCATCCCTCGGCACCCAGACCTATATC
TGCAACGTCAACCACAAACCTTCCAACACCAAAGTGGACAAGAAAGTGGAACCTAAGTCCTGC
GATAAGACTCATACTTGCCCTCCTTGTCCAGCACCAGAGCTGCTGGGGGGGCCAAGCGTGTTT
CTCTTTCCACCTAAGCCTAAAGACACCCTGATGATCTCCAGGACCCCAGAGGTGACATGTGTG
GTGGTGGACGTGTCTCATGAGGACCCTGAGGTGAAATTCAATTGGTATGTGGACGGCGTTGAG
GTTCACAACGCAAAGACCAAGCCAAGGGAGGAGCAGTATAATAGCACCTATCGCGTGGTGTCC
GTCCTGACAGTGCTGCACCAGGACTGGCTGAACGGGAAGGAGTATAAGTGTAAAGTGAGCAAC
AAGGCACTGCCTGCTCCTATCGAGAAGACTATCAGCAAAGCTAAAGGACAGCCAAGAGAGCCC
CAGGTGACCTACCTGCCACCTTCTCGGGACGAACTGACCAAAAACCAGGTGAGCCTGACTTGC
CTGGTGAAGGGCTTTTATCCCTCTGATATTGCAGTGGAGTGGGAGAGTAACGGGCAGCCCGAG
AACAACTACAAGACTACTCCACCAGTTCTGGATTCCGACGGCAGCTTCTTCCTGTATAGCAAA
CTGACAGTGGACAAGTCCCGGTGGCAGCAGGGCAACGTTTTTAGCTGCAGCGTGATGCATGAG
GCTCTGCACAACCATTACACACAGAAGTCTCTGTCTCTGTCCCCCGGAAAGTGA
(SEQ ID NO: 2)
Secretory sequence (Bold)
Anti-IL6 Heavy Chain SEQ ID NO: 3
ATGGCCACCGGGTCTCGGACAAGCCTCCTGCTCGCATTCGGGCTCCTGTGTCTGCCTTGGCTG
CAAGAAGGATCCGCATTTCCCACCATTCCACTGTCTGAGGTGCAGCTGGTCGAGTCTGGAGGA
GGACTGGTGCAGCCTGGCAGGTCTCTGAGGCTGTCTTGCGCTGCCAGCCGGTTTACCTTTGAT
GATTACGCCATGCACTGGGTGAGGCAGGCTCCCGGCAAAGGACTGGAATGGGTGTCCGGAATT
TCCTGGAATAGTGGCAGGATCGGCTATGCCGACTCTGTCAAAGGCCGGTTTACAATCTCCCGC
GACAACGCTGAGAACTCCCTGTTCCTGCAGATGAACGGCCTGAGAGCTGAAGATACCGCCCTG
TACTATTGCGCCAAGGGGCGCGACAGCTTCGACATTTGGGGCCAGGGAACCATGGTGACTGTG
AGCAGCGCATCCACAAAAGGGCCCTCCGTGTTCCCCCTGGCACCTTCCAGTAAATCCACTTCT
GGCGGAACAGCAGCTCTCGGCTGTCTGGTGAAGGATTATTTCCCCGAGCCAGTGACAGTGTCT
TGGAATTCTGGCGCACTCACCAGTGGAGTCCACACTTTTCCAGCCGTGCTGCAGAGCTCCGGA
CTGTATTCCCTGAGCTCCGTCGTGACAGTGCCATCCTCTTCTCTGGGAACTCAGACATATATT
TGCAACGTTAATCATAAGCCTTCTAACACCAAGGTGGATAAGAAAGTGGAGCCCAAATCCTGC
GACAAGACACACACTTGCCCACCATGCCCTGCCCCTGAACTGCTGGGAGGACCAAGCGTGTTT
CTCTTCCCTCCTAAGCCTAAGGATACCCTGATGATCTCTAGGACCCCAGAGGTGACATGCGTG
GTGGTTGACGTCTCCCATGAAGATCCTGAAGTGAAATTTAACTGGTACGTGGACGGAGTGGAA
GTGCACAATGCAAAGACCAAACCCCGCGAGGAACAGTACAACTCCACTTACCGGGTGGTTTCC
GTGCTGACAGTGCTGCACCAGGATTGGCTGAACGGAAAAGAGTATAAGTGCAAAGTGAGCAAT
AAGGCCCTGCCCGCCCCTATTGAGAAGACCATCTCTAAGGCTAAAGGCCAGCCTCGCGAACCC
CAGGTTACCTATCTGCCTCCAAGCAGAGATGAGCTCACCAAAAACCAGGTGTCTCTGACCTGT
CTGGTGAAAGGCTTCTATCCAAGCGATATCGCCGTGGAGTGGGAGTCCAACGGACAGCCAGAA
AACAATTACAAGACTACCCCACCTGTCCTGGACAGCGACGGGAGCTTCTTTCTGTACTCTAAG
CTGACAGTCGACAAAAGCCGGTGGCAGCAAGGCAACGTCTTCAGCTGCAGCGTCATGCACGAG
GCCCTGCATAATCATTATACTCAGAAGTCTCTGAGCCTGAGCCCTGGCAAGTAG
Secretory sequence (Bold)
(SEQ ID NO: 3)
Anti-IL6 Heavy Chain SEQ ID NO: 4
ATGGCCACTGGAAGCAGAACCTCCCTGCTGCTGGCATTCGGACTGCTGTGCCTGCCATGGCTG
CAGGAGGGATCCGCTTTCCCAACCATCCCCCTCAGCGAGGTGCAGCTCGTTGAATCTGGAGGA
GGACTGGTGCAACCAGGACGCTCCCTGAGACTGTCTTGTGCTGCTTCCAGGTTTACTTTTGAC
GATTATGCTATGCACTGGGTGAGACAGGCCCCAGGAAAAGGACTGGAATGGGTGTCTGGAATT
TCTTGGAACAGCGGACGCATTGGCTACGCCGACTCTGTGAAGGGAAGGTTTACTATCTCCAGG
GATAACGCGGAAAACTCCCTCTTCCTCCAGATGAACGGCCTGAGGGCAGAGGACACCGCTCTG
TACTACTGCGCCAAAGGAAGAGATAGCTTCGATATCTGGGGACAGGGGACCATGGTGACAGTT
TCCAGCGCTAGCACCAAGGGCCCCAGCGTGTTCCCACTGGCCCCATCCTCCAAGAGCACTTCT
GGCGGGACTGCTGCACTGGGCTGCCTGGTGAAGGATTATTTCCCTGAGCCTGTGACAGTGAGC
TGGAACTCAGGAGCACTGACTTCCGGGGTGCATACATTCCCCGCTGTGCTGCAGTCTTCTGGG
CTGTATTCCCTCAGCAGCGTGGTGACCGTCCCTTCCTCAAGCCTGGGAACCCAGACATATATT
TGTAACGTGAACCACAAGCCAAGCAATACAAAGGTGGATAAGAAGGTGGAGCCTAAGTCCTGT
GACAAAACACACACATGTCCCCCATGTCCAGCTCCTGAACTGCTTGGCGGACCATCCGTCTTC
CTGTTTCCACCCAAACCAAAGGATACACTGATGATCAGCCGGACACCAGAGGTGACTTGCGTC
GTCGTGGACGTCAGCCATGAAGACCCCGAGGTGAAGTTTAATTGGTATGTGGACGGGGTGGAG
GTCCACAACGCCAAGACCAAACCCAGGGAGGAGCAGTACAACTCCACTTATCGCGTGGTTTCT
GTGCTGACAGTCCTGCACCAGGATTGGCTGAACGGGAAGGAGTACAAATGCAAAGTGTCCAAT
AAGGCCCTGCCCGCCCCAATCGAGAAAACTATTTCAAAGGCCAAAGGACAGCCCAGAGAGCCA
CAGGTGACCTACCTCCCTCCTTCCAGGGACGAGCTCACTAAGAATCAGGTGTCTCTGACTTGC
CTGGTGAAAGGCTTTTATCCTTCTGACATCGCAGTGGAGTGGGAGAGCAATGGCCAGCCCGAG
AACAATTATAAAACAACACCACCCGTCCTGGACTCTGATGGCAGCTTTTTCCTGTATAGCAAG
CTGACAGTGGACAAATCACGCTGGCAGCAGGGGAATGTCTTCAGCTGTAGCGTGATGCACGAA
GCTCTGCACAATCACTATACACAGAAGTCCCTGTCCCTGAGCCCAGGAAAATAA
SEQ ID (NO: 4)
Secretory sequence (Bold)
Anti-IL6 Heavy Chain SEQ ID NO: 5
ATGGCTACCGGCAGCAGGACTAGCCTGCTGCTGGCTTTCGGCCTGCTGTGTCTGCCTTGGCTG
CAAGAGGGGTCCGCTTTCCCTACTATCCCTCTGTCCGAAGTGCAGCTGGTCGAGAGCGGAGGG
GGCCTGGTGCAGCCTGGAAGAAGTCTGCGCCTGTCCTGCGCAGCAAGCAGGTTTACATTTGAC
GACTACGCAATGCACTGGGTGCGCCAAGCTCCAGGCAAAGGCTTAGAATGGGTGTCTGGCATC
AGCTGGAACTCAGGGCGGATCGGCTACGCAGACAGCGTGAAGGGCAGGTTCACTATCTCTAGG
GACAACGCCGAGAACTCCCTGTTCCTGCAGATGAATGGGCTGCGGGCAGAAGACACTGCACTG
TATTATTGTGCTAAGGGGAGAGACTCTTTCGACATCTGGGGCCAGGGCACAATGGTGACTGTG
TCCTCTGCCTCTACCAAGGGCCCTTCCGTGTTCCCACTGGCACCAAGCAGCAAATCCACATCC
GGGGGGACCGCAGCTCTCGGATGTCTGGTGAAAGACTATTTCCCTGAGCCCGTCACAGTGTCT
TGGAATTCCGGCGCCCTGACAAGCGGCGTGCACACTTTTCCTGCCGTTCTGCAGAGCTCCGGC
CTATACTCCCTGTCCAGCGTGGTGACAGTCCCTTCTAGCAGTCTGGGCACACAGACTTATATT
TGCAACGTGAATCACAAGCCATCTAACACCAAGGTGGATAAGAAGGTGGAACCAAAGTCCTGT
GATAAAACCCATACCTGTCCTCCATGTCCAGCTCCTGAACTCCTGGGGGGACCCTCTGTGTTC
CTGTTCCCACCTAAGCCTAAAGACACTCTGATGATTTCCAGAACTCCTGAGGTGACTTGCGTG
GTGGTGGATGTGTCCCATGAGGATCCTGAGGTCAAGTTCAACTGGTACGTGGACGGAGTCGAG
GTGCATAACGCTAAAACTAAACCAAGAGAGGAACAGTATAATTCCACTTATAGAGTTGTGAGC
GTCCTGACCGTGCTGCACCAGGACTGGCTGAACGGAAAAGAATACAAGTGTAAGGTGTCCAAC
AAGGCACTCCCCGCACCAATTGAGAAGACCATTTCCAAGGCCAAGGGCCAGCCAAGAGAGCCT
CAGGTGACCTATCTGCCTCCAAGCCGGGACGAACTGACAAAGAATCAGGTCAGCCTGACTTGC
CTGGTGAAGGGGTTTTACCCTTCTGACATCGCCGTGGAATGGGAGTCTAATGGACAGCCCGAA
AACAACTACAAGACCACACCACCCGTGCTGGACAGCGATGGCTCCTTTTTCCTGTATAGTAAA
CTGACCGTCGACAAGTCTCGCTGGCAGCAGGGCAACGTGTTTTCTTGCAGCGTTATGCATGAA
GCCCTCCACAACCACTATACACAGAAAAGCCTGTCTCTCAGCCCTGGGAAGTGA (SEQ ID
NO: 5)
Secretory sequence (Bold)
Anti-IL6 Light Chain SEQ ID NO: 6
ATGGCCACTGGAAGCCGGACAAGCCTGCTGCTGGCCTTTGGCCTGCTGTGTCTGCCTTGGCTG
CAGGAGGGAAGCGCATTTCCAACAATTCCTCTGAGCGACATTCAGATGACACAGAGCCCCAGC
AGCGTGTCCGCATCAGTGGGAGACAGGGTGACTATCACATGTAGAGCTTCTCAAGGAATTAGC
TCTTGGCTGGCCTGGTATCAGCAGAAGCCAGGCAAGGCCCCCAAGCTGCTGATCTATGGAGCT
AGCTCTCTGGAGTCTGGGGTGCCATCTAGGTTCAGTGGCTCCGGCAGCGGAACAGACTTCACA
CTGACTATCAGCAGCCTGCAGCCTGAGGACTTTGCCAGCTACTACTGCCAGCAGGCAAATAGC
TTTCCCTATACTTTCGGACAGGGCACCAAGCTGGAGATTAAGCGGACCGTTGCTGCTCCAAGC
GTGTTCATCTTCCCACCTTCCGACGAGCAGCTGAAGTCTGGCACCGCCAGCGTGGTGTGTCTG
CTGAACAATTTCTATCCCCGTGAAGCCAAAGTGCAGTGGAAGGTGGATAACGCTCTCCAGTCT
GGCAATTCCCAGGAGAGCGTGACAGAGCAGGATTCTAAGGATTCTACCTACTCCCTGTCCAGC
ACACTGACCCTGAGCAAGGCCGATTACGAAAAACACAAAGTGTACGCCTGCGAAGTCACACAC
CAGGGGCTGAGCTCCCCAGTGACAAAGAGCTTTAATAGAGGGGAGTGCTGA (SEQ ID NO:
6)
Secretory sequence (Bold)
Anti-IL6 Light Chain SEQ ID NO: 7
ATGGCTACAGGGAGCCGCACTAGCCTGCTGCTGGCTTTTGGCCTGCTGTGCCTGCCATGGCTG
CAAGAGGGGTCCGCCTTTCCTACCATCCCCCTGTCCGATATTCAGATGACCCAGTCCCCTAGC
AGCGTGTCTGCCAGCGTGGGAGACAGGGTGACTATCACCTGTAGGGCCAGCCAGGGCATTTCT
AGCTGGCTGGCTTGGTACCAGCAGAAGCCAGGAAAGGCTCCCAAACTGCTGATCTACGGGGCA
TCCTCTCTGGAGTCCGGAGTGCCAAGCAGATTCTCTGGGAGCGGCAGCGGGACCGATTTCACA
CTGACCATTAGCAGCCTGCAGCCAGAAGACTTCGCCAGCTACTATTGTCAGCAGGCAAACTCT
TTTCCTTATACCTTCGGGCAGGGGACTAAACTGGAAATCAAGCGGACAGTGGCTGCTCCAAGC
GTGTTCATCTTCCCACCTTCCGACGAGCAGCTGAAGTCTGGCACAGCTTCCGTGGTGTGCCTG
CTGAACAACTTTTATCCAAGGGAAGCTAAAGTGCAGTGGAAGGTGGACAACGCTCTGCAGTCT
GGAAATAGCCAGGAATCCGTGACTGAGCAGGATAGCAAAGACAGCACATACAGCCTGTCTTCC
ACTCTGACCCTGAGCAAGGCAGACTACGAGAAACACAAAGTGTATGCCTGTGAGGTGACCCAT
CAGGGCCTGTCTAGCCCAGTGACCAAGTCCTTTAACAGAGGCGAATGTTGA (SEQ ID NO:
7)
Secretory sequence (Bold)
Anti-IL6 Light Chain SEQ ID NO: 8
ATGGCAACTGGATCCCGGACCTCTCTGCTGCTGGCCTTCGGACTGCTGTGCCTGCCATGGCTG
CAGGAGGGGAGCGCTTTTCCTACTATCCCCCTGTCTGACATCCAGATGACTCAGAGCCCAAGC
TCTGTGTCCGCAAGCGTGGGGGACAGGGTGACAATCACTTGCAGGGCATCCCAGGGAATCTCC
TCTTGGCTGGCATGGTACCAGCAGAAACCTGGAAAAGCCCCAAAACTGCTGATTTATGGCGCT
TCCAGCCTCGAATCCGGAGTGCCATCCCGGTTTTCTGGCTCCGGCAGCGGGACAGATTTTACT
CTGACCATCTCTAGCCTGCAGCCAGAGGATTTTGCCTCCTATTATTGCCAGCAGGCCAACAGC
TTTCCTTATACCTTTGGACAGGGAACTAAGCTGGAGATCAAGAGGACAGTGGCTGCTCCTAGC
GTGTTCATCTTCCCACCTTCTGACGAACAGCTGAAGTCTGGAACAGCCTCTGTGGTGTGCCTC
CTCAACAACTTCTATCCCCGGGAGGCTAAGGTCCAGTGGAAAGTGGACAACGCCCTGCAGTCC
GGAAACTCCCAGGAGAGCGTGACCGAGCAGGACAGCAAGGATAGCACTTATTCCCTGTCCTCC
ACCCTGACTCTGTCCAAGGCCGACTACGAAAAGCACAAAGTGTACGCCTGCGAAGTCACTCAT
CAGGGACTGAGCTCCCCCGTGACCAAGAGCTTCAATAGGGGAGAATGTTAG (SEQ ID NO:
8)
Secretory sequence (Bold)
Anti-IL6 Light Chain SEQ ID NO: 9
ATGGCAACTGGCTCCAGGACTAGCCTGCTGCTGGCATTTGGCCTCCTGTGTCTGCCATGGCTG
CAGGAGGGCTCCGCCTTCCCAACAATTCCACTGTCCGACATCCAGATGACACAGTCCCCTAGC
AGCGTGAGCGCCTCCGTGGGAGATAGAGTGACAATTACCTGTCGCGCAAGCCAGGGGATCAGC
AGCTGGCTGGCCTGGTATCAACAGAAACCTGGAAAAGCCCCCAAGCTCCTGATCTATGGCGCC
AGCAGCCTGGAAAGCGGGGTTCCAAGCCGGTTTTCCGGGTCCGGCAGCGGAACTGACTTCACC
CTGACAATTTCAAGCCTGCAGCCCGAGGATTTTGCAAGCTACTACTGTCAGCAGGCTAATAGC
TTTCCTTACACATTCGGCCAGGGCACCAAGCTCGAAATTAAAAGAACTGTGGCTGCCCCATCC
GTGTTTATCTTCCCACCCTCTGACGAACAGCTGAAGTCCGGGACAGCCTCTGTGGTGTGCCTG
CTGAACAATTTTTACCCCAGGGAGGCTAAGGTCCAATGGAAGGTCGACAATGCTCTGCAGTCT
GGAAACTCCCAGGAGTCTGTGACTGAGCAGGACAGCAAGGACAGCACCTATAGCCTGTCTTCC
ACCCTGACCCTGAGCAAGGCCGATTACGAAAAGCACAAGGTGTATGCCTGTGAGGTGACCCAC
CAGGGACTGTCTAGCCCAGTGACTAAATCCTTTAATAGAGGCGAATGCTGA (SEQ ID NO:
9)
Secretory sequence (Bold)
Anti-IL4Rα Heavy Chain SEQ ID NO: 10
ATGGCCACAGGCTCTAGGACATCCCTGCTGCTGGCCTTCGGACTGCTGTGTCTGCCTTGGCTG
CAGGAGGGATCTGCATTCCCAACTATCCCTCTGTCCGAGGTTCAGCTGGTGGAAAGCGGGGGA
GGGCTGGAGCAGCCTGGGGGGTCCCTGAGACTGTCCTGCGCTGGATCCGGCTTCACTTTTCGC
GATTATGCCATGACATGGGTGCGGCAGGCCCCCGGCAAAGGACTGGAGTGGGTTTCCAGCATT
TCTGGCAGCGGAGGGAACACCTACTATGCCGATAGCGTGAAGGGAAGGTTTACAATCAGCCGC
GATAACAGCAAGAATACCCTCTATCTGCAGATGAATTCTCTGAGGGCAGAGGACACTGCCGTG
TATTATTGCGCAAAGGATAGGCTGAGCATCACTATCCGCCCACGCTACTACGGGCTGGACGTG
TGGGGGCAGGGAACTACCGTTACCGTGTCTTCCGCCAGCACAAAGGGACCTTCTGTGTTCCCC
CTGGCTCCCTGTAGCAGATCCACCTCTGAGAGCACCGCTGCCCTGGGATGCCTGGTGAAGGAT
TATTTCCCAGAGCCCGTGACTGTGAGCTGGAATTCAGGCGCACTCACCTCTGGGGTGCACACC
TTCCCTGCCGTGCTGCAGTCCAGCGGCCTGTATTCTCTCTCCAGCGTCGTGACCGTGCCTTCC
AGTAGCCTGGGAACTAAAACATATACCTGTAACGTGGATCACAAGCCCTCCAATACCAAGGTG
GACAAGCGGGTCGAGAGCAAGTACGGACCCCCATGTCCTCCCTGTCCAGCTCCTGAGTTCCTG
GGGGGCCCTTCAGTGTTCCTGTTTCCCCCTAAGCCAAAGGACACTCTCATGATCTCCAGGACT
CCAGAGGTGACATGCGTGGTGGTGGATGTCAGCCAGGAGGATCCAGAGGTCCAGTTCAATTGG
TACGTCGACGGGGTGGAGGTGCATAACGCCAAGACTAAGCCCCGCGAGGAACAGTTTAATTCC
ACTTACAGGGTGGTCTCTGTGCTGACTGTCCTGCATCAGGATTGGCTGAACGGAAAGGAGTAT
AAGTGCAAAGTGTCTAATAAGGGGCTGCCCAGCTCCATCGAGAAAACAATCTCTAAGGCTAAG
GGGCAGCCTCGGGAGCCTCAGGTGTACACTCTGCCCCCTTCACAGGAAGAGATGACCAAAAAT
CAGGTGTCCCTGACTTGCCTGGTGAAGGGGTTTTATCCCTCTGACATCGCAGTGGAATGGGAG
TCCAACGGCCAGCCTGAAAACAACTATAAGACAACCCCTCCCGTGCTGGATAGCGACGGGAGC
TTTTTCCTGTACAGCAGACTGACTGTGGATAAATCTAGGTGGCAGGAGGGAAACGTGTTTTCT
TGCAGCGTCATGCACGAAGCCCTGCACAATCACTACACACAGAAATCCCTGTCCCTGTCCCTG
GGCTGA (SEQ ID NO: 10)
Secretory sequence (Bold)
Anti-IL4Rα Heavy Chain SEQ ID NO: 11
ATGGCTACCGGGTCCAGGACATCTCTGCTGCTGGCCTTCGGACTGCTGTGCCTGCCATGGCTG
CAGGAAGGCTCAGCCTTTCCAACAATCCCACTGTCCGAAGTGCAGCTGGTGGAGAGCGGCGGC
GGCCTTGAACAACCTGGAGGCTCTTTGAGACTGTCATGCGCCGGGTCCGGATTTACCTTTCGC
GACTACGCAATGACTTGGGTGCGCCAGGCTCCCGGAAAGGGACTGGAATGGGTTTCCTCTATT
AGCGGGTCCGGCGGCAACACTTATTACGCAGATAGCGTGAAGGGGCGCTTCACTATTAGCAGG
GACAATTCTAAAAACACCCTGTACCTGCAGATGAACAGCTTAAGAGCCGAAGACACAGCTGTG
TACTACTGCGCTAAAGACAGACTCTCCATTACAATCCGCCCAAGGTATTACGGCCTGGACGTG
TGGGGCCAGGGAACAACAGTGACCGTGAGCTCTGCTTCCACTAAGGGCCCTAGCGTGTTCCCC
CTGGCTCCATGCTCCCGCAGCACATCAGAGTCTACCGCCGCACTGGGATGTCTGGTGAAGGAT
TACTTCCCCGAGCCTGTGACTGTGAGCTGGAATAGCGGGGCCCTGACCTCTGGAGTTCATACA
TTCCCAGCCGTGCTGCAGTCTTCCGGCCTGTACTCTCTGAGCTCTGTGGTGACCGTCCCATCC
TCTTCTCTGGGCACAAAGACCTACACATGTAACGTTGACCACAAGCCATCCAATACCAAGGTG
GACAAGAGAGTGGAATCCAAGTATGGCCCTCCTTGTCCCCCTTGTCCTGCTCCAGAGTTCCTG
GGAGGGCCATCCGTCTTCCTCTTCCCTCCCAAGCCTAAGGATACACTGATGATCTCCAGGACC
CCTGAAGTGACATGTGTCGTGGTGGACGTGAGCCAAGAAGACCCCGAGGTGCAGTTCAACTGG
TACGTGGACGGAGTCGAGGTGCACAACGCTAAAACAAAGCCCCGCGAGGAGCAGTTCAACTCC
ACATACCGGGTGGTCTCAGTGCTGACTGTGCTTCATCAGGATTGGCTGAATGGGAAGGAGTAC
AAGTGCAAGGTGAGCAACAAGGGACTGCCATCTAGCATCGAGAAAACAATCAGCAAGGCTAAG
GGACAGCCAAGGGAACCTCAGGTGTATACTCTGCCACCCTCCCAGGAAGAGATGACTAAGAAT
CAGGTCTCCCTGACCTGTCTGGTGAAGGGATTCTACCCTAGCGACATTGCTGTCGAGTGGGAG
TCCAACGGGCAGCCAGAAAATAATTACAAGACCACACCTCCAGTGCTGGACAGCGATGGATCC
TTCTTCCTGTACTCTCGGCTGACCGTGGATAAGAGCCGGTGGCAGGAGGGCAACGTTTTCTCT
TGCAGCGTGATGCACGAGGCTCTGCATAATCACTATACACAGAAGTCTCTAAGCCTGTCTCTG
GGATGA (SEQ ID NO: 11)
Secretory sequence (Bold)
Anti-IL4Rα Heavy Chain SEQ ID NO: 12
ATGGCTACAGGATCCCGGACTAGCCTGCTGCTGGCCTTCGGCCTGTTGTGCCTGCCTTGGCTG
CAGGAGGGGTCTGCCTTTCCAACAATCCCACTGTCTGAGGTCCAGCTGGTGGAGTCCGGCGGA
GGGCTAGAACAGCCTGGGGGATCTCTGAGGCTCTCTTGCGCAGGATCCGGCTTTACATTCAGA
GACTACGCAATGACTTGGGTCAGACAGGCCCCTGGAAAGGGGCTGGAGTGGGTTTCCAGCATT
TCCGGATCCGGGGGCAACACATATTACGCTGACTCTGTGAAGGGCAGGTTCACAATCAGCAGG
GATAACTCCAAGAACACCCTCTATCTGCAGATGAACTCCCTGCGGGCCGAGGATACCGCAGTG
TACTACTGTGCCAAAGATAGGCTGAGCATCACAATCCGCCCTAGGTATTATGGGCTCGACGTG
TGGGGCCAGGGAACTACAGTGACAGTGTCCTCGGCATCCACCAAAGGCCCCTCCGTTTTCCCC
CTGGCACCCTGTAGCCGCTCTACTTCTGAGAGTACTGCTGCCCTGGGCTGCCTGGTGAAGGAT
TACTTTCCAGAGCCCGTCACAGTGTCCTGGAATTCTGGGGCTCTGACTTCTGGCGTGCACACA
TTCCCCGCAGTGCTGCAGTCTTCTGGCCTGTACTCTCTGTCTTCTGTGGTGACCGTGCCCAGC
AGCAGCCTGGGCACTAAGACATATACCTGTAATGTTGACCACAAACCTTCCAACACTAAGGTG
GACAAGAGGGTGGAGTCTAAGTATGGACCTCCCTGTCCACCTTGTCCTGCTCCAGAGTTCCTC
GGGGGACCAAGCGTTTTCCTGTTCCCCCCAAAGCCAAAGGACACTCTGATGATTAGCCGCACT
CCCGAAGTGACTTGTGTTGTGGTGGACGTCTCTCAGGAGGATCCTGAGGTGCAGTTTAATTGG
TACGTGGATGGCGTGGAGGTGCACAACGCCAAGACAAAACCACGGGAGGAACAGTTCAATAGC
ACCTATAGGGTCGTGTCCGTCCTGACAGTGCTCCACCAGGATTGGCTCAACGGAAAAGAATAC
AAATGCAAGGTGTCTAACAAGGGGCTGCCTTCCAGCATCGAGAAGACTATTAGCAAGGCAAAG
GGGCAGCCAAGAGAGCCTCAGGTGTATACCCTGCCCCCATCTCAGGAGGAGATGACAAAGAAC
CAGGTCTCCCTGACTTGTCTGGTCAAGGGGTTCTACCCATCTGACATCGCTGTGGAGTGGGAG
AGCAACGGCCAACCCGAGAATAACTACAAAACAACCCCACCCGTGCTGGACAGCGATGGATCC
TTCTTCCTGTATTCCAGGTTGACCGTGGACAAATCTCGCTGGCAGGAGGGAAACGTTTTCTCT
TGCTCCGTGATGCACGAGGCCCTGCACAATCACTACACACAGAAATCTCTCTCTCTGTCTCTG
GGGTGA (SEQ ID NO: 12)
Secretory sequence (Bold)
Anti-IL4Rα Heavy Chain SEQ ID NO: 13
ATGGCTACAGGGTCTCGGACAAGTCTGCTGCTGGCATTCGGGCTGCTGTGCCTGCCATGGCTG
CAAGAGGGAAGCGCATTCCCAACCATTCCACTCAGCGAGGTGCAGCTGGTCGAAAGCGGGGGG
GGACTGGAACAACCTGGAGGATCCCTGCGGCTGTCATGCGCAGGCTCCGGCTTTACCTTCAGG
GACTACGCCATGACATGGGTGAGACAGGCTCCTGGGAAGGGGCTCGAGTGGGTGAGCAGCATT
TCCGGAAGCGGGGGAAACACCTATTACGCAGATAGTGTTAAGGGCCGCTTTACTATCTCTAGG
GACAATTCCAAGAACACCCTGTACCTGCAGATGAACAGCCTGCGAGCCGAGGACACCGCAGTG
TATTACTGTGCCAAGGACCGGCTCTCTATTACCATTAGACCTAGGTATTACGGGCTGGACGTG
TGGGGACAGGGAACAACAGTGACCGTGTCTTCTGCCTCCACAAAAGGGCCCTCTGTGTTCCCT
CTGGCACCTTGCTCCAGGTCTACCTCCGAGAGCACAGCTGCACTGGGATGTCTGGTTAAAGAT
TACTTTCCAGAACCAGTTACTGTGAGCTGGAACTCTGGAGCTCTGACCTCCGGAGTTCACACA
TTCCCTGCAGTGCTGCAGTCTAGCGGCCTGTATTCCCTGTCCTCCGTCGTGACCGTGCCTTCC
TCCTCTCTGGGCACTAAGACCTACACTTGCAACGTGGATCACAAACCTAGCAATACAAAGGTC
GATAAACGGGTTGAGAGCAAATACGGCCCTCCATGTCCTCCTTGTCCAGCCCCTGAATTCCTG
GGCGGACCCTCCGTTTTCCTGTTCCCACCCAAGCCCAAGGACACACTGATGATTTCTAGGACT
CCTGAAGTGACATGCGTGGTCGTGGATGTCTCCCAGGAGGATCCAGAAGTCCAGTTCAATTGG
TACGTGGATGGAGTGGAGGTGCACAATGCCAAGACAAAGCCAAGGGAGGAGCAGTTTAACTCT
ACTTACAGAGTGGTGAGCGTGCTCACAGTGCTGCATCAGGATTGGCTCAACGGAAAAGAGTAC
AAGTGTAAGGTCAGCAATAAGGGCCTGCCATCCTCCATTGAGAAAACCATCTCCAAGGCAAAG
GGGCAGCCAAGAGAACCTCAGGTCTACACCCTGCCACCATCTCAAGAGGAGATGACCAAGAAT
CAGGTGAGCCTCACTTGCCTGGTGAAGGGATTCTACCCTAGCGACATTGCCGTGGAGTGGGAA
TCTAACGGGCAGCCAGAGAACAACTACAAGACAACTCCTCCCGTGCTGGATAGCGACGGGTCT
TTCTTCCTGTATAGCAGGCTGACAGTGGATAAGAGCCGCTGGCAAGAGGGCAACGTCTTTTCT
TGTTCCGTCATGCACGAGGCTCTGCATAACCACTATACCCAGAAGTCACTGTCCCTCTCCCTG
GGGTG (SEQ ID NO: 13)
Secretory sequence (Bold)
Anti-IL4Rα Light Chain SEQ ID NO: 14
ATGGCCACAGGCTCTAGGACATCCCTGCTGCTGGCCTTCGGACTGCTGTGTCTGCCTTGGCTG
CAGGAGGGATCTGCATTCCCAACTATCCCTCTGTCCGATATTGTGATGACCCAGAGCCCCCTG
AGCCTGCCAGTGACTCCTGGGGAGCCCGCATCTATCAGCTGCCGGTCCTCTCAGTCTCTGCTG
TATTCTATCGGGTACAACTACCTGGATTGGTACCTGCAGAAAAGTGGGCAGAGCCCCCAGCTG
CTCATCTATCTGGGGTCCAACAGGGCTAGTGGCGTGCCAGACCGGTTCTCCGGATCCGGCTCC
GGAACAGACTTTACACTGAAAATTAGCCGCGTGGAGGCCGAGGACGTGGGGTTTTATTATTGT
ATGCAGGCCCTGCAGACCCCATACACATTTGGCCAGGGGACAAAGCTGGAAATTAAGCGCACT
GTGGCCGCTCCGTCTGTGTTCATCTTTCCTCCCAGCGATGAACAGCTGAAGTCTGGGACCGCT
AGCGTCGTGTGCCTGCTGAACAATTTTTACCCCAGGGAGGCTAAGGTGCAGTGGAAGGTGGAC
AATGCCCTGCAGAGCGGGAACAGCCAGGAGAGTGTTACTGAGCAGGATTCTAAAGATTCCACC
TATTCCCTGTCTTCCACCCTGACTCTGTCTAAGGCCGATTACGAAAAACATAAGGTGTACGCA
TGCGAGGTGACCCACCAGGGGCTGAGCTCTCCCGTGACTAAGAGCTTCAATCGCGGAGAGTGC
TGA (SEQ ID NO: 14)
Secretory sequence (Bold)
Anti-IL4Rα Light Chain SEQ ID NO: 15
ATGGCTACAGGCAGCAGAACCAGCCTGCTGCTGGCATTTGGCCTGCTGTGCCTGCCTTGGCTG
CAGGAGGGGAGCGCTTTTCCCACAATTCCTCTGTCTGATATCGTCATGACCCAATCTCCCCTG
TCCCTGCCTGTGACTCCAGGAGAGCCCGCTAGCATTTCTTGCAGGTCTTCCCAGAGCCTGCTG
TACAGCATCGGCTATAACTACCTGGATTGGTATCTGCAGAAAAGCGGGCAGTCTCCTCAGCTG
CTGATCTACCTGGGCTCTAACAGAGCCTCTGGGGTCCCCGACAGGTTTTCCGGAAGCGGCTCT
GGCACCGACTTTACTCTCAAAATCAGCCGCGTGGAGGCAGAGGACGTGGGCTTCTATTACTGC
ATGCAGGCCCTGCAGACACCATATACATTCGGACAGGGGACCAAGCTGGAGATTAAGAGAACA
GTGGCTGCCCCAAGCGTGTTTATCTTTCCTCCCTCCGATGAACAGCTGAAAAGCGGCACTGCT
TCCGTGGTGTGCCTGCTGAATAATTTCTACCCTAGAGAGGCCAAAGTCCAGTGGAAGGTGGAC
AACGCCCTGCAGAGCGGGAACAGCCAGGAAAGTGTCACCGAGCAGGATTCCAAGGATTCCACA
TATTCTCTGTCCAGCACTCTGACACTGTCCAAGGCAGACTACGAAAAACACAAGGTCTACGCC
TGCGAAGTGACCCACCAGGGACTGTCTAGCCCTGTGACTAAGTCTTTTAATAGGGGGGAGTGT
TAG (SEQ ID NO: 15)
Secretory sequence (Bold)
Anti-IL4Rα Light Chain SEQ ID NO: 16
ATGGCTACTGGCAGCAGAACCAGCCTGCTGCTGGCATTCGGGCTGCTCTGCCTGCCATGGCTG
CAGGAGGGATCCGCCTTCCCAACTATCCCCCTGAGCGATATCGTGATGACCCAGTCTCCCCTG
AGCCTGCCAGTTACACCCGGCGAACCTGCTAGCATCAGCTGCAGATCCTCCCAGTCTCTCCTG
TACTCCATCGGGTACAATTATCTGGATTGGTATCTGCAGAAGTCTGGCCAATCCCCCCAGCTG
CTGATCTACCTGGGCTCCAACAGAGCAAGCGGCGTGCCCGATAGATTCAGCGGCAGCGGGAGC
GGCACTGATTTTACTCTGAAGATCAGCAGGGTGGAGGCCGAAGATGTGGGATTTTACTACTGC
ATGCAAGCACTGCAGACTCCTTACACATTCGGCCAGGGAACTAAGCTGGAGATCAAAAGAACC
GTGGCAGCTCCAAGCGTCTTCATTTTCCCACCTTCTGACGAGCAGCTGAAGTCCGGCACAGCT
TCCGTCGTGTGCCTCCTGAACAACTTCTACCCCAGGGAGGCAAAGGTGCAATGGAAAGTGGAC
AACGCTCTGCAGAGCGGAAACAGTCAGGAGTCCGTGACCGAGCAGGACAGCAAAGACTCCACT
TACAGCCTGAGCTCTACTCTGACCCTGAGCAAAGCTGACTACGAGAAGCATAAGGTGTATGCT
TGCGAGGTCACCCACCAGGGCCTCTCTTCTCCCGTGACCAAGAGCTTCAACAGAGGCGAGTGC
TGA (SEQ ID NO: 16)
Secretory sequence (Bold)
Anti-IL4Rα Light Chain SEQ ID NO: 17
ATGGCAACTGGAAGCAGGACCTCCCTGCTCCTGGCTTTCGGCCTGCTCTGTCTGCCATGGCTG
CAAGAAGGATCTGCCTTTCCTACAATTCCACTGTCCGACATCGTGATGACACAGTCCCCCCTG
TCTCTGCCTGTCACCCCAGGCGAACCAGCCTCTATTTCTTGTCGGTCCTCTCAGTCCCTGCTG
TATAGCATCGGATATAATTATCTGGACTGGTACCTGCAAAAATCCGGCCAGTCTCCTCAGCTG
CTGATCTATCTGGGCTCCAACCGGGCTAGCGGAGTCCCAGACCGGTTTTCCGGGTCTGGCAGT
GGGACAGATTTTACACTGAAAATTTCCCGGGTGGAGGCTGAGGACGTGGGATTTTACTACTGT
ATGCAGGCCCTGCAAACCCCATATACTTTCGGACAGGGAACAAAGCTGGAGATCAAAAGAACC
GTGGCCGCCCCCAGCGTTTTCATCTTCCCACCAAGCGACGAGCAGCTCAAATCTGGGACCGCT
AGCGTGGTCTGTCTGCTGAATAACTTCTACCCAAGGGAAGCAAAGGTGCAGTGGAAGGTCGAC
AACGCACTGCAGAGCGGGAACTCCCAGGAGAGCGTGACTGAACAGGACAGCAAGGACAGCACC
TATAGCCTCAGCAGCACTCTGACCCTGTCTAAAGCTGATTACGAAAAACACAAGGTGTATGCT
TGTGAAGTGACTCACCAGGGCCTGTCTTCCCCTGTTACAAAGTCCTTCAATAGAGGAGAATGT
TAA (SEQ ID NO: 17)
Secretory sequence (Bold)

Exemplary heavy and light chain antibody sequences are provided in the table below.

TABLE 2
Heavy and light chain Anti-IL6 and Anti-IL4Rα antibody sequences

Anti-IL6 Heavy Chain SEQ ID NO: 18
VKGRFTISRDNAENSLFLQMNGLRAEDTALYYCAKGRDSFDIWGQGTMVTVSSASTKGPSVFP
LAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNG
KEYKCKVSNKALPAPIEKTISKAKGQPREPQVTYLPPSRDELTKNQVSLTCLVKGFYPSDIAV
EWESNGQPENNYKTTPPVLDSDGSFFLYSKLTVDKSRWQQGNVFSCSVMHEALHNHYTQKSLS
LSPGK (SEQ ID NO: 18)
Anti-IL6 Light Chain SEQ ID NO: 19
DIQMTQSPSSVSASVGDRVTITCRASQGISSWLAWYQQKPGKAPKLLIYGASSLESGVPSRFS
GSGSGTDFTLTISSLQPEDFASYYCQQANSFPYTFGQGTKLEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 19)
Anti-IL4Rα Heavy Chain SEQ ID NO: 20
EVQLVESGGGLEQPGGSLRLSCAGSGFTFRDYAMTWVRQAPGKGLEWVSSISGSGGNTYYADS
VKGRFTISRDNSKNTLYLQMNSLRAEDTAVYYCAKDRLSITIRPRYYGLDVWGQGTTVTVSSA
STKGPSVFPLAPCSRSTSESTAALGCLVKDYFPEPVTVSWNSGALTSGVHTFPAVLQSSGLYS
LSSVVTVPSSSLGTKTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHY
TQKSLSLSLG (SEQ ID NO: 20)
Anti-IL4Rα Light Chain SEQ ID NO: 21
DIVMTQSPLSLPVTPGEPASISCRSSQSLLYSIGYNYLDWYLQKSGQSPQLLIYLGSNRASGV
PDRFSGSGSGTDFTLKISRVEAEDVGFYYCMQALQTPYTFGQGTKLEIKRTVAAPSVFIFPPS
DEQLKSGTASVVCLLNNFYPREAKVQWKVDNALQSGNSQESVTEQDSKDSTYSLSSTLTLSKA
DYEKHKVYACEVTHQGLSSPVTKSFNRGEC (SEQ ID NO: 21)
Anti-IL6 Heavy Chain with secretory sequence-SEQ ID NO: 22
DYAMHWVRQAPGKGLEWVSGISWNSGRIGYADSVKGRFTISRDNAENSLFLQMNGLRAEDTAL
YYCAKGRDSFDIWGQGTMVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAKGQPREP
QVTYLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK*
(SEQ ID NO: 22)
(Secretory sequence in bold)
Anti-IL6 Light Chain with secretory sequence SEQ ID NO: 23
MATGSRTSLLLAFGLLCLPWLQEGSAFPTIPLSDIQMTQSPSSVSASVGDRVTITCRASQGIS
SWLAWYQQKPGKAPKLLIYGASSLESGVPSRFSGSGSGTDFTLTISSLQPEDFASYYCQQANS
FPYTFGQGTKLEIKRTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVTKSFNRGEC*
(SEQ ID NO: 23)
(Secretory sequence in bold)
Anti-IL4Rα Heavy Chain with secretory sequence SEQ ID NO: 24
MATGSRTSLLLAFGLLCLPWLQEGSAFPTIPLSEVQLVESGGGLEQPGGSLRLSCAGSGFTFR
DYAMTWVRQAPGKGLEWVSSISGSGGNTYYADSVKGRFTISRDNSKNTLYLQMNSLRAEDTAV
YYCAKDRLSITIRPRYYGLDVWGQGTTVTVSSASTKGPSVFPLAPCSRSTSESTAALGCLVKD
YFPEPVTVSWNSGALTSGVHTFPAVLQSSGLYSLSSVVTVPSSSLGTKTYTCNVDHKPSNTKV
DKRVESKYGPPCPPCPAPEFLGGPSVFLFPPKPKDTLMISRTPEVTCVVVDVSQEDPEVQFNW
YVDGVEVHNAKTKPREEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLG (SEQ ID NO: 24)
(Secretory sequence in bold)
Anti-IL4Rα Light Chain with secretory sequence SEQ ID NO: 25
MATGSRTSLLLAFGLLCLPWLQEGSAFPTIPLSDIVMTQSPLSLPVTPGEPASISCRSSQSLL
YSIGYNYLDWYLQKSGQSPQLLIYLGSNRASGVPDRFSGSGSGTDFTLKISRVEAEDVGFYYC
MQALQTPYTFGQGTKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGLSSPVTKSFNRGEC
(SEQ ID NO: 25)
(Secretory sequence in bold)

As used herein, the term “antibody” encompasses both intact antibody and antibody fragment. Typically, an intact “antibody” is an immunoglobulin that binds specifically to a particular antigen. An antibody may be a member of any immunoglobulin class, including any of the human classes: IgG, IgM, IgE, IgA, and IgD. Typically, an intact antibody is a tetramer. Each tetramer is composed of two identical pairs of polypeptide chains, each pair having one “light” (approximately 25 kD) and one “heavy” chain (approximately 50-70 kD). The N-terminus of each chain defines a variable region of about 100 to 110 or more amino acids primarily responsible for antigen recognition. The terms “variable light chain” (VL) and “variable heavy chain” (VH) refer to these corresponding regions on the light and heavy chain respectively. Each variable region can be further subdivided into hypervariable (HV) and framework (FR) regions. The hypervariable regions comprise three areas of hypervariability sequence called complementarity determining regions (CDR 1, CDR 2 and CDR 3), separated by four framework regions (FR1, FR2, FR2, and FR4) which form a beta-sheet structure and serve as a scaffold to hold the HV regions in position. The C-terminus of each heavy and light chain defines a constant region consisting of one domain for the light chain (CL) and three for the heavy chain (CH1, CH2 and CH3). A light chain of immunoglobulins can be further differentiated into the isotypes kappa and lambda.

In some embodiments, the terms “intact antibody” or “fully assembled antibody” are used in reference to an antibody that contains two heavy chains and two light chains, optionally associated by disulfide bonds as occurs with naturally-produced antibodies. In some embodiments, an antibody according to the present invention is an antibody fragment.

In some embodiments, the present invention can be used to deliver an “antibody fragment.” As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules; and multi specific antibodies formed from antibody fragments. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region. In many embodiments, an antibody fragment contains a sufficient sequence of the parent antibody of which it is a fragment that it binds to the same antigen as does the parent antibody; in some embodiments, a fragment binds to the antigen with a comparable affinity to that of the parent antibody and/or competes with the parent antibody for binding to the antigen. Examples of antigen binding fragments of an antibody include, but are not limited to, Fab fragment, Fab′ fragment, F(ab′)2 fragment, scFv fragment, Fv fragment, dsFv diabody, dAb fragment, Fd′ fragment, Fd fragment, and an isolated complementarity determining region (CDR).

The present invention may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bi-specific antibodies.

### Synthesis of mRNA

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

### Synthesis of mRNA using SP6 RNA Polymerase

In some embodiments, mRNA is produced using SP6 RNA Polymerase. SP6 RNA Polymerase is a DNA-dependent RNA polymerase with high sequence specificity for SP6 promoter sequences. The SP6 polymerase catalyzes the 5′→3′ in vitro synthesis of RNA on either single-stranded DNA or double-stranded DNA downstream from its promoter; it incorporates native ribonucleotides and/or modified ribonucleotides and/or labeled ribonucleotides into the polymerized transcript. Examples of such labeled ribonucleotides include biotin-, fluorescein-, digoxigenin-, aminoallyl-, and isotope-labeled nucleotides.

The sequence for bacteriophage SP6 RNA polymerase was initially described (GenBank: Y00105.1) as having the following amino acid sequence:

(SEQ ID NO: 1)
MQDLHAIQLQLEEEMFNGGIRRFEADQQRQIAAGSESDTAWNRRLLSEL
IAPMAEGIQAYKEEYEGKKGRAPRALAFLQCVENEVAAYITMKVVMDML
NTDATLQAIAMSVAERIEDQVRFSKLEGHAAKYFEKVKKSLKASRTKSY
RHAHNVAVVAEKSVAEKDADFDRWEAWPKETQLQIGTTLLEILEGSVFY
NGEPVFMRAMRTYGGKTIYYLQTSESVGQWISAFKEHVAQLSPAYAPCV
IPPRPWRTPFNGGFHTEKVASRIRLVKGNREHVRKLTQKQMPKVYKAIN
ALQNTQWQINKDVLAVIEEVIRLDLGYGVPSFKPLIDKENKPANPVPVE
FQHLRGRELKEMLSPEQWQQFINWKGECARLYTAETKRGSKSAAVVRMV
GQARKYSAFESIYFVYAMDSRSRVYVQSSTLSPQSNDLGKALLRFTEGR
PVNGVEALKWFCINGANLWGWDKKTFDVRVSNVLDEEFQDMCRDIAADP
LTFTQWAKADAPYEFLAWCFEYAQYLDLVDEGRADEFRTHLPVHQDGSC
SGIQHYSAMLRDEVGAKAVNLKPSDAPQDIYGAVAQVVIKKNALYMDAD
DATTFTSGSVTLSGTELRAMASAWDSIGITRSLTKKPVMTLPYGSTRLT
CRESVIDYIVDLEEKEAQKAVAEGRTANKVHPFEDDRQDYLTPGAAYNY
MTALIWPSISEVVKAPIVAMKMIRQLARFAAKRNEGLMYTLPTGFILEQ
KIMATEMLRVRTCLMGDIKMSLQVETDIVDEAAMMGAAAPNFVHGHDAS
HLILTVCELVDKGVTSIAVIHDSFGTHADNTLTLRVALKGQMVAMYIDG
NALQKLLEEHEVRWMVDTGIEVPEQGEFDLNEIMDSEYVFA.

An SP6 RNA polymerase suitable for the present invention can be any enzyme having substantially the same polymerase activity as bacteriophage SP6 RNA polymerase. Thus, in some embodiments, an SP6 RNA polymerase suitable for the present invention may be modified from SEQ ID NO: 1. For example, a suitable SP6 RNA polymerase may contain one or more amino acid substitutions, deletions, or additions. In some embodiments, a suitable SP6 RNA polymerase has an amino acid sequence about 99%, 98%, 97%, 96%, 95%, 94%, 93%, 92%, 91%, 90%, 89%, 88%, 87%, 86%, 85%, 84%, 83%, 82%, 81%, 80%, 75%, 70%, 65%, or 60% identical or homologous to SEQ ID NO: 1. In some embodiments, a suitable SP6 RNA polymerase may be a truncated protein (from N-terminus, C-terminus, or internally) but retain the polymerase activity. In some embodiments, a suitable SP6 RNA polymerase is a fusion protein.

An SP6 RNA polymerase suitable for the invention may be a commercially-available product, e.g., from Aldevron, Ambion, New England Biolabs (NEB), Promega, and Roche. The SP6 may be ordered and/or custom designed from a commercial source or a non-commercial source according to the amino acid sequence of SEQ ID NO: 1 or a variant of SEQ ID NO: 1 as described herein. The SP6 may be a standard-fidelity polymerase or may be a high-fidelity/high-efficiency/high-capacity which has been modified to promote RNA polymerase activities, e.g., mutations in the SP6 RNA polymerase gene or post-translational modifications of the SP6 RNA polymerase itself. Examples of such modified SP6 include SP6 RNA Polymerase-Plus™ from Ambion, HiScribe SP6 from NEB, and RiboMAX™ and Riboprobe® Systems from Promega.

In some embodiments, a suitable SP6 RNA polymerase is a fusion protein. For example, an SP6 RNA polymerase may include one or more tags to promote isolation, purification, or solubility of the enzyme. A suitable tag may be located at the N-terminus, C-terminus, and/or internally. Non-limiting examples of a suitable tag include Calmodulin-binding protein (CBP); Fasciola hepatica 8-kDa antigen (Fh8); FLAG tag peptide; glutathione-S-transferase (GST); Histidine tag (e.g., hexahistidine tag (His6)); maltose-binding protein (MBP); N-utilization substance (NusA); small ubiquitin related modifier (SUMO) fusion tag; Streptavidin binding peptide (STREP); Tandem affinity purification (TAP); and thioredoxin (TrxA). Other tags may be used in the present invention. These and other fusion tags have been described, e.g., Costa et al. Frontiers in Microbiology 5 (2014): 63 and in PCT/US16/57044, the contents of which are incorporated herein by reference in their entireties. In certain embodiments, a His tag is located at SP6's N-terminus.

DNA Template

Typically, a DNA template is either entirely double-stranded or mostly single-stranded with a double-stranded SP6 promoter sequence.

Linearized plasmid DNA (linearized via one or more restriction enzymes), linearized genomic DNA fragments (via restriction enzyme and/or physical means), PCR products, and/or synthetic DNA oligonucleotides can be used as templates for in vitro transcription with SP6, provided that they contain a double-stranded SP6 promoter upstream (and in the correct orientation) of the DNA sequence to be transcribed.

In some embodiments, the linearized DNA template has a blunt-end.

In some embodiments, the DNA sequence to be transcribed may be optimized to facilitate more efficient transcription and/or translation. For example, the DNA sequence may be optimized regarding cis-regulatory elements (e.g., TATA box, termination signals, and protein binding sites), artificial recombination sites, chi sites, CpG dinucleotide content, negative CpG islands, GC content, polymerase slippage sites, and/or other elements relevant to transcription; the DNA sequence may be optimized regarding cryptic splice sites, mRNA secondary structure, stable free energy of mRNA, repetitive sequences, RNA instability motif, and/or other elements relevant to mRNA processing and stability; the DNA sequence may be optimized regarding codon usage bias, codon adaptability, internal chi sites, ribosomal binding sites (e.g., IRES), premature polyA sites, Shine-Dalgarno (SD) sequences, and/or other elements relevant to translation; and/or the DNA sequence may be optimized regarding codon context, codon-anticodon interaction, translational pause sites, and/or other elements relevant to protein folding. Optimization methods known in the art may be used in the present invention, e.g., GeneOptimizer by ThermoFisher and OptimumGene™, which are described in US 20110081708, the contents of which are incorporated herein by reference in its entirety.

In some embodiments, the DNA template includes a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

Exemplary 3′ and/or 5′ UTR sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides' resistance to in vivo nuclease digestion.

Large-Scale mRNA Synthesis.

In some embodiments, the present invention can be used in large-scale production of stable LNP encapsulated mRNA. In some embodiments, a method according to the invention synthesizes mRNA at least 100 mg, 150 mg, 200 mg, 300 mg, 400 mg, 500 mg, 600 mg, 700 mg, 800 mg, 900 mg, 1 g, 5 g, 10 g, 25 g, 50 g, 75 g, 100 g, 250 g, 500 g, 750 g, 1 kg, 5 kg, 10 kg, 50 kg, 100 kg, 1000 kg, or more at a single batch. As used herein, the term “batch” refers to a quantity or amount of mRNA synthesized at one time, e.g., produced according to a single manufacturing setting. A batch may refer to an amount of mRNA synthesized in one reaction that occurs via a single aliquot of enzyme and/or a single aliquot of DNA template for continuous synthesis under one set of conditions. mRNA synthesized at a single batch would not include mRNA synthesized at different times that are combined to achieve the desired amount. Generally, a reaction mixture includes SP6 RNA polymerase, a linear DNA template, and an RNA polymerase reaction buffer (which may include ribonucleotides or may require addition of ribonucleotides).

According to the present invention, 1-100 mg of SP6 polymerase is typically used per gram (g) of mRNA produced. In some embodiments, about 1-90 mg, 1-80 mg, 1-60 mg, 1-50 mg, 1-40 mg, 10-100 mg, 10-80 mg, 10-60 mg, 10-50 mg of SP6 polymerase is used per gram of mRNA produced. In some embodiments, about 5-20 mg of SP6 polymerase is used to produce about 1 gram of mRNA. In some embodiments, about 0.5 to 2 grams of SP6 polymerase is used to produce about 100 grams of mRNA. In some embodiments, about 5 to 20 grams of SP6 polymerase is used to about 1 kilogram of mRNA. In some embodiments, at least 5 mg of SP6 polymerase is used to produce at least 1 gram of mRNA. In some embodiments, at least 500 mg of SP6 polymerase is used to produce at least 100 grams of mRNA. In some embodiments, at least 5 grams of SP6 polymerase is used to produce at least 1 kilogram of mRNA. In some embodiments, about 10 mg, 20 mg, 30 mg, 40 mg, 50 mg, 60 mg, 70 mg, 80 mg, 90 mg, or 100 mg of plasmid DNA is used per gram of mRNA produced. In some embodiments, about 10-30 mg of plasmid DNA is used to produce about 1 gram of mRNA. In some embodiments, about 1 to 3 grams of plasmid DNA is used to produce about 100 grams of mRNA. In some embodiments, about 10 to 30 grams of plasmid DNA is used to about 1 kilogram of mRNA. In some embodiments, at least 10 mg of plasmid DNA is used to produce at least 1 gram of mRNA. In some embodiments, at least 1 gram of plasmid DNA is used to produce at least 100 grams of mRNA. In some embodiments, at least 10 grams of plasmid DNA is used to produce at least 1 kilogram of mRNA.

In some embodiments, the concentration of the SP6 RNA polymerase in the reaction mixture may be from about 1 to 100 nM, 1 to 90 nM, 1 to 80 nM, 1 to 70 nM, 1 to 60 nM, 1 to 50 nM, 1 to 40 nM, 1 to 30 nM, 1 to 20 nM, or about 1 to 10 nM. In certain embodiments, the concentration of the SP6 RNA polymerase is from about 10 to 50 nM, 20 to 50 nM, or 30 to 50 nM. A concentration of 100 to 10000 Units/ml of the SP6 RNA polymerase may be used, as examples, concentrations of 100 to 9000 Units/ml, 100 to 8000 Units/ml, 100 to 7000 Units/ml, 100 to 6000 Units/ml, 100 to 5000 Units/ml, 100 to 1000 Units/ml, 200 to 2000 Units/ml, 500 to 1000 Units/ml, 500 to 2000 Units/ml, 500 to 3000 Units/ml, 500 to 4000 Units/ml, 500 to 5000 Units/ml, 500 to 6000 Units/ml, 1000 to 7500 Units/ml, and 2500 to 5000 Units/ml may be used.

The concentration of each ribonucleotide (e.g., ATP, UTP, GTP, and CTP) in a reaction mixture is between about 0.1 mM and about 10 mM, e.g., between about 1 mM and about 10 mM, between about 2 mM and about 10 mM, between about 3 mM and about 10 mM, between about 1 mM and about 8 mM, between about 1 mM and about 6 mM, between about 3 mM and about 10 mM, between about 3 mM and about 8 mM, between about 3 mM and about 6 mM, between about 4 mM and about 5 mM. In some embodiments, each ribonucleotide is at about 5 mM in a reaction mixture. In some embodiments, the total concentration of rNTPs (for example, ATP, GTP, CTP and UTPs combined) used in the reaction range between 1 mM and 40 mM. In some embodiments, the total concentration of rNTPs (for example, ATP, GTP, CTP and UTPs combined) used in the reaction range between 1 mM and 30 mM, or between 1 mM and 28 mM, or between 1 mM to 25 mM, or between 1 mM and 20 mM. In some embodiments, the total rNTPs concentration is less than 30 mM. In some embodiments, the total rNTPs concentration is less than 25 mM. In some embodiments, the total rNTPs concentration is less than 20 mM. In some embodiments, the total rNTPs concentration is less than 15 mM. In some embodiments, the total rNTPs concentration is less than 10 mM.

The RNA polymerase reaction buffer typically includes a salt/buffering agent, e.g., Tris, HEPES, ammonium sulfate, sodium bicarbonate, sodium citrate, sodium acetate, potassium phosphate sodium phosphate, sodium chloride, and magnesium chloride.

The pH of the reaction mixture may be between about 6 to 8.5, from 6.5 to 8.0, from 7.0 to 7.5, and in some embodiments, the pH is 7.5.

Linear or linearized DNA template (e.g., as described above and in an amount/concentration sufficient to provide a desired amount of RNA), the RNA polymerase reaction buffer, and SP6 RNA polymerase are combined to form the reaction mixture. The reaction mixture is incubated at between about 37° C. and about 42° C. for thirty minutes to six hours, e.g., about sixty to about ninety minutes.

In some embodiments, about 5 mM NTPs, about 0.05 mg/mL SP6 polymerase, and about 0.1 mg/ml DNA template in a suitable RNA polymerase reaction buffer (final reaction mixture pH of about 7.5) is incubated at about 37° C. to about 42° C. for sixty to ninety minutes.

In some embodiments, a reaction mixture contains linearized double stranded DNA template with an SP6 polymerase-specific promoter, SP6 RNA polymerase, RNase inhibitor, pyrophosphatase, 29 mM NTPs, 10 mM DTT and a reaction buffer (when at 10× is 800 mM HEPES, 20 mM spermidine, 250 mM MgCl₂, pH 7.7) and quantity sufficient (QS) to a desired reaction volume with RNase-free water; this reaction mixture is then incubated at 37° C. for 60 minutes. The polymerase reaction is then quenched by addition of DNase I and a DNase I buffer (when at 10× is 100 mM Tris-HCl, 5 mM MgCl₂ and 25 mM CaCl₂), pH 7.6) to facilitate digestion of the double-stranded DNA template in preparation for purification. This embodiment has been shown to be sufficient to produce 100 grams of mRNA.

In some embodiments, a reaction mixture includes NTPs at a concentration ranging from 1-10 mM, DNA template at a concentration ranging from 0.01-0.5 mg/ml, and SP6 RNA polymerase at a concentration ranging from 0.01-0.1 mg/ml, e.g., the reaction mixture comprises NTPs at a concentration of 5 mM, the DNA template at a concentration of 0.1 mg/ml, and the SP6 RNA polymerase at a concentration of 0.05 mg/ml.

Nucleotides

Various naturally-occurring or modified nucleosides may be used to product mRNA according to the present invention. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the mRNA comprises one or more nonstandard nucleotide residues. The nonstandard nucleotide residues may include, e.g., 5-methyl-cytidine (“5mC”), pseudouridine (“ψU”), and/or 2-thio-uridine (“2sU”). See, e.g., U.S. Pat. No. 8,278,036 or WO2011012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US20120195936 and international publication WO2011012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modifications may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in US 2016/0040154, US 2015/0376220, PCT application PCT/US18/19954 entitled “METHODS FOR PURIFICATION OF MESSENGER RNA” filed on Feb. 27, 2018, and PCT application PCT/US18/19978 entitled “METHODS FOR PURIFICATION OF MESSENGER RNA” filed on Feb. 27, 2018, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of mRNA

Full-length or abortive transcripts of mRNA may be detected and quantified using any methods available in the art. In some embodiments, the synthesized mRNA molecules are detected using blotting, capillary electrophoresis, chromatography, fluorescence, gel electrophoresis, HPLC, silver stain, spectroscopy, ultraviolet (UV), or UPLC, or a combination thereof. Other detection methods known in the art are included in the present invention. In some embodiments, the synthesized mRNA molecules are detected using UV absorption spectroscopy with separation by capillary electrophoresis. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

In some embodiments, mRNA generated by the method disclosed herein comprises less than 10%, less than 9%, less than 8%, less than 7%, less than 6%, less than 5%, less than 4%, less than 3%, less than 2%, less than 1%, less than 0.5%, less than 0.1% impurities other than full length mRNA. The impurities include IVT contaminants, e.g., proteins, enzymes, free nucleotides and/or shortmers.

In some embodiments, mRNA produced according to the invention is substantially free of shortmers or abortive transcripts. In particular, mRNA produced according to the invention contains undetectable level of shortmers or abortive transcripts by capillary electrophoresis or Glyoxal gel electrophoresis. As used herein, the term “shortmers” or “abortive transcripts” refers to any transcripts that are less than full-length. In some embodiments, “shortmers” or “abortive transcripts” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail.

mRNA Solution.

In some embodiments, mRNA may be provided in a solution to be mixed with a lipid solution such that the mRNA may be encapsulated in lipid nanoparticles. A suitable mRNA solution may be any aqueous solution containing mRNA to be encapsulated at various concentrations. For example, a suitable mRNA solution may contain an mRNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable mRNA solution may contain an mRNA at a concentration ranging from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.09 mg/ml, 0.08 mg/ml, 0.07 mg/ml, 0.06 mg/ml, or 0.05 mg/ml.

Typically, a suitable mRNA solution may also contain a buffering agent and/or salt. Generally, buffering agents can include HEPES, ammonium sulfate, sodium bicarbonate, sodium citrate, sodium acetate, potassium phosphate and sodium phosphate. In some embodiments, suitable concentration of the buffering agent may range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM. In some embodiments, suitable concentration of the buffering agent is or greater than about 0.1 mM, 0.5 mM, 1 mM, 2 mM, 4 mM, 6 mM, 8 mM, 10 mM, 15 mM, 20 mM, 25 mM, 30 mM, 35 mM, 40 mM, 45 mM, or 50 mM.

Exemplary salts can include sodium chloride, magnesium chloride, and potassium chloride. In some embodiments, suitable concentration of salts in an mRNA solution may range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM. Salt concentration in a suitable mRNA solution is or greater than about 1 mM, 5 mM, 10 mM, 20 mM, 30 mM, 40 mM, 50 mM, 60 mM, 70 mM, 80 mM, 90 mM, or 100 mM.

In some embodiments, a suitable mRNA solution may have a pH ranging from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5. In some embodiments, a suitable mRNA solution may have a pH of or no greater than about 3.5, 4.0, 4.1, 4.2, 4.3, 4.4, 4.5, 4.6, 4.7, 4.8, 4.9, 5.0, 5.2, 5.4, 5.6, 5.8, 6.0, 6.1, 6.3, and 6.5.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20×greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

### Delivery Vehicles

The stable lipid nanoparticles formulations described here are suitable as delivery vehicles for mRNA.

As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired mRNA to a target cell or tissue. In some embodiments, a nanoparticle delivery vehicle is a liposome. In some embodiments, a liposome comprises one or more cationic lipids, one or more non-cationic lipids, one or more cholesterol-based lipids and one or more PEG-modified lipids. In some embodiments, a liposome comprises no more than three distinct lipid components. In some embodiments, one distinct lipid component is a sterol-based cationic lipid.

Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

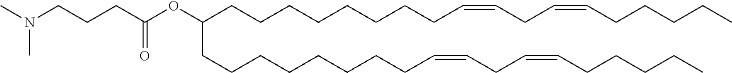 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

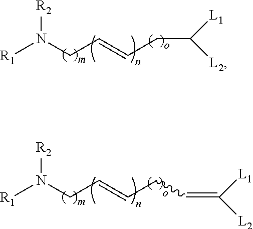

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)—N,N-dimethyl-6-(9Z,12Z)-octadeca-9,12-dien-1-yl) tetracosa-15,18-dien-1-amine (“HGT5000”), having a compound structure of:

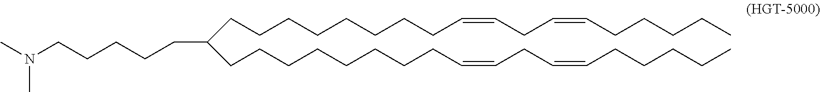 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)—N,N-dimethyl-6-((9Z,12Z)-octadeca-9,12-dien-1-yl) tetracosa-4,15,18-trien-1-amine (“HGT5001”), having a compound structure of:

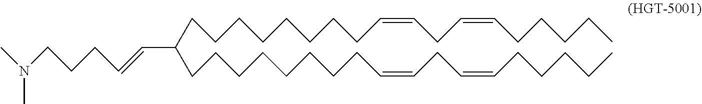 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)—N,N-dimethyl-6-((9Z,12Z)-octadeca-9,12-dien-1-yl) tetracosa-5,15,18-trien- 1-amine (“HGT5002”), having a compound structure of:

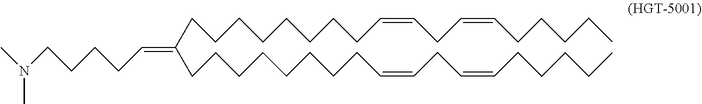 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

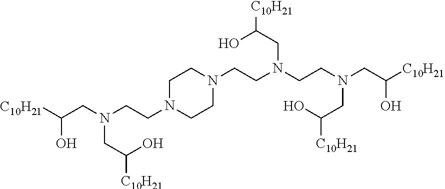

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

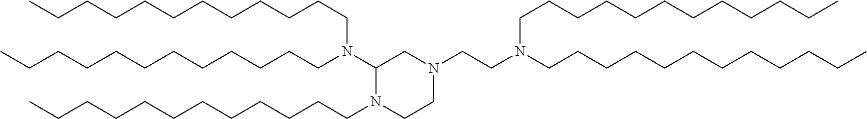 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

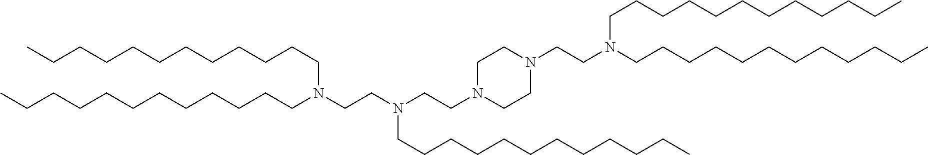 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

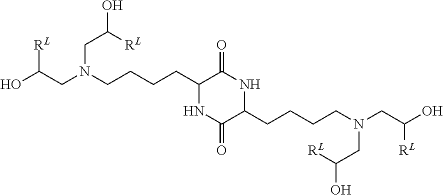

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

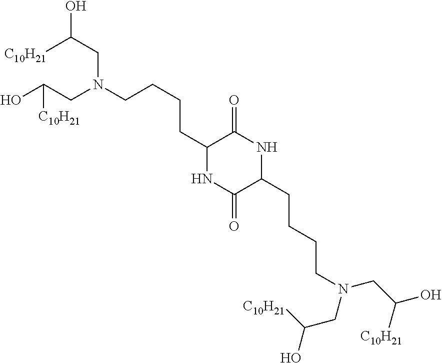

and pharmaceutically acceptable salts thereof. In certain embodiments, the compositions and methods of the present invention include a cationic lipid having a compound structure of:

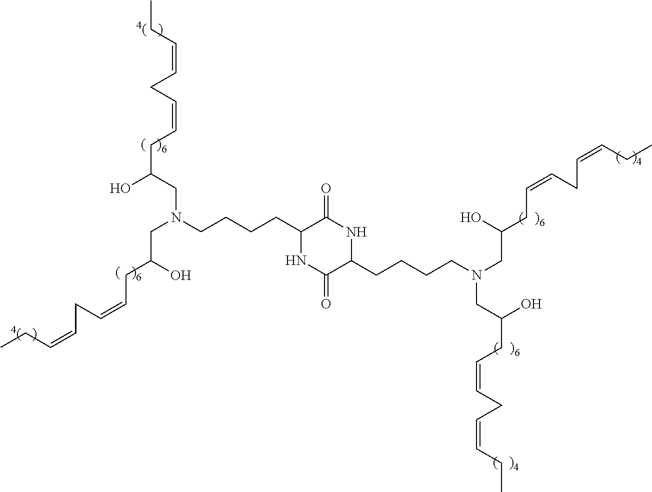

and pharmaceutically acceptable salts thereof. In certain embodiments, the compositions and methods of the present invention include a cationic lipid having a compound structure of:

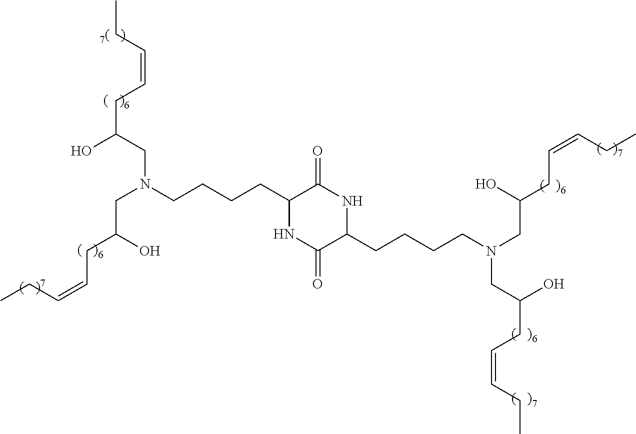

and pharmaceutically acceptable salts thereof. In certain embodiments, the compositions and methods of the present invention include a cationic lipid having a compound structure of:

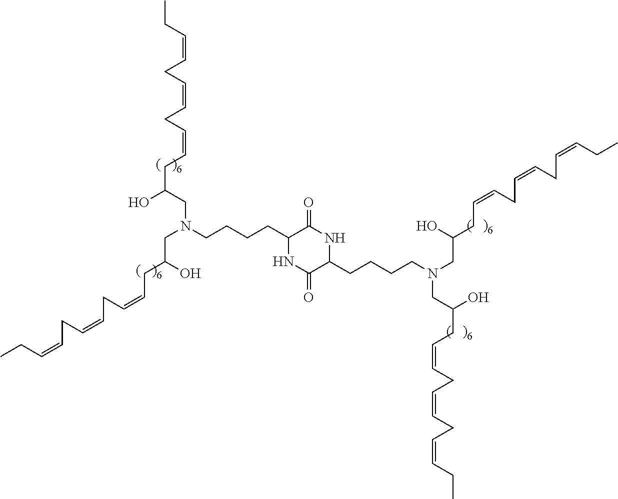

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

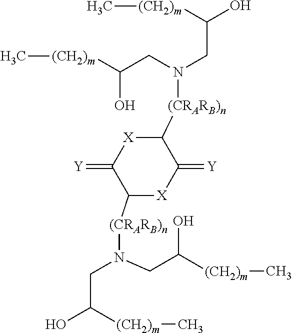

or a pharmaceutically acceptable salt thereof, wherein each X independently is 0 or S; each Y independently is 0 or S; each m independently is 0 to 20; each n independently is 1 to 6; each RA is independently hydrogen, optionally substituted C₁-50 alkyl, optionally substituted C₂-50 alkenyl, optionally substituted C₂-50 alkynyl, optionally substituted C₃-10 carbocyclyl, optionally substituted 3-14 membered heterocyclyl, optionally substituted C₆-14 aryl, optionally substituted 5-14 membered heteroaryl or halogen; and each RB is independently hydrogen, optionally substituted C₁-50 alkyl, optionally substituted C₂-50 alkenyl, optionally substituted C₂-50 alkynyl, optionally substituted C₃-10 carbocyclyl, optionally substituted 3-14 membered heterocyclyl, optionally substituted C₆-14 aryl, optionally substituted 5-14 membered heteroaryl or halogen. In certain embodiments, the compositions and methods of the present invention include a cationic lipid, “Target 23”, having a compound structure of:

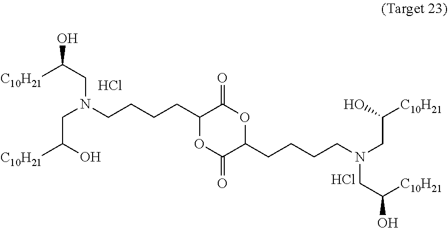

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

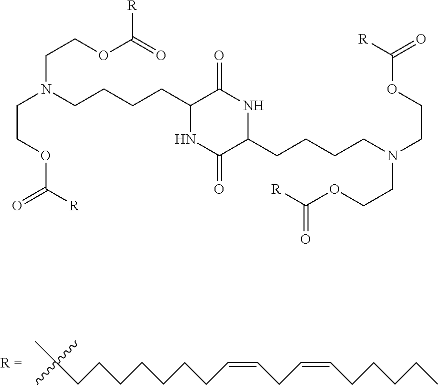

or a pharmaceutically acceptable salt thereof. In some embodiments, the compositions and methods of the present invention include a cationic lipid having the compound structure:

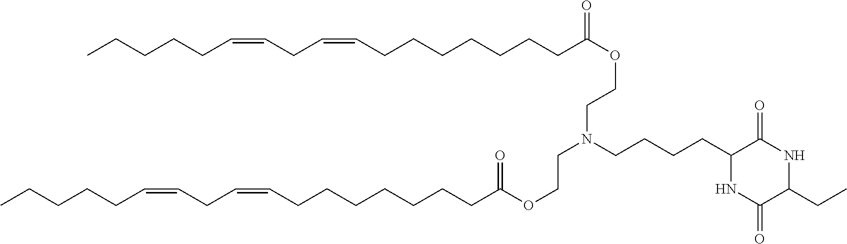 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

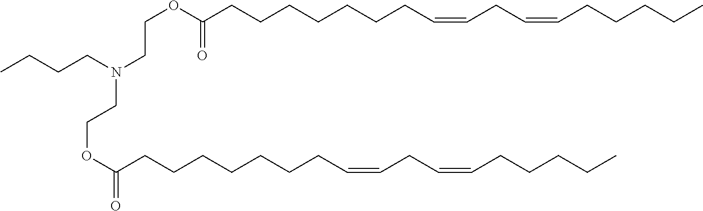 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

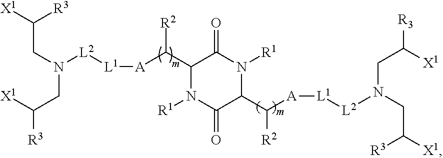

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

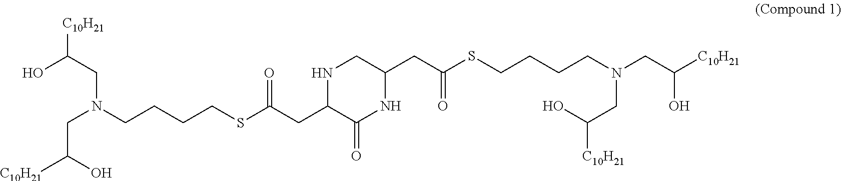

or a pharmaceutically acceptable salt thereof. In some embodiments, the compositions and methods of the present invention include a cationic lipid of the following formula:

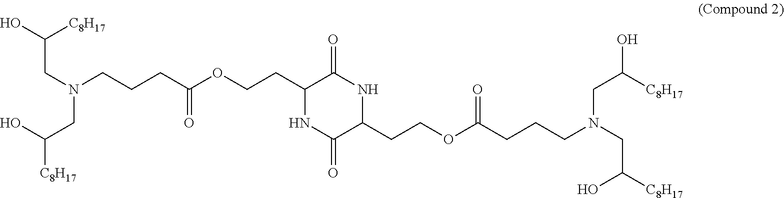

or a pharmaceutically acceptable salt thereof. In some embodiments, the compositions and methods of the present invention include a cationic lipid of the following formula:

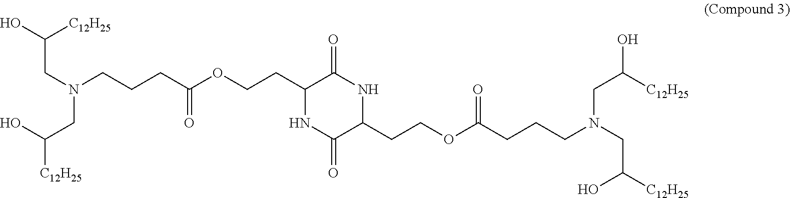

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

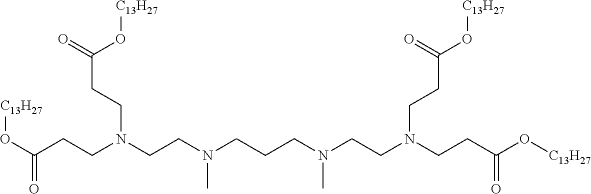

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

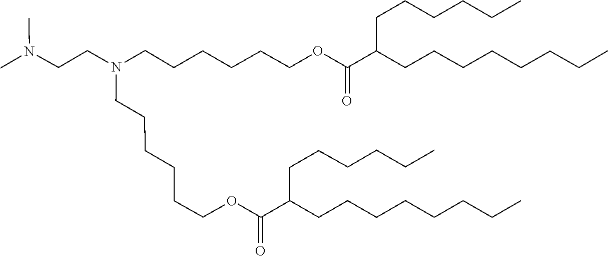 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

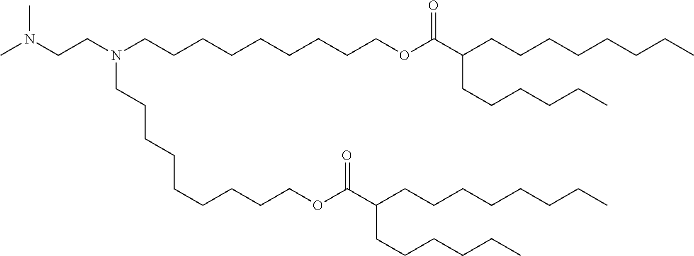 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

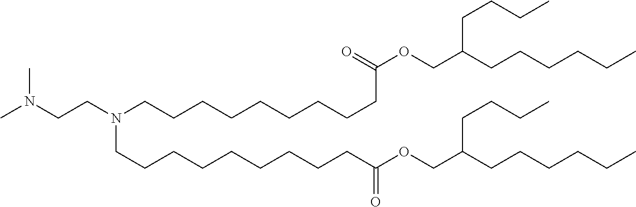 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

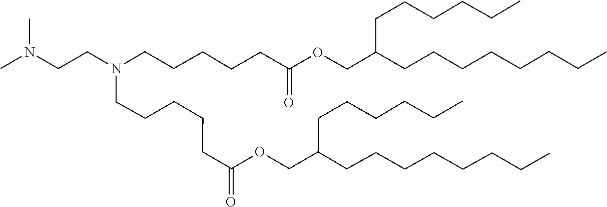 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

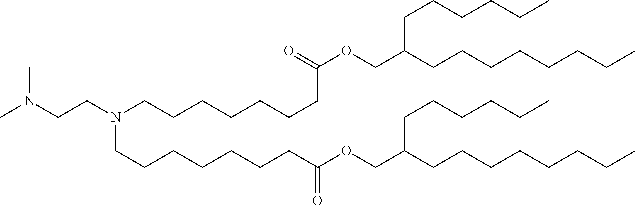 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

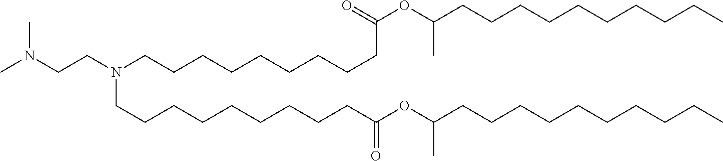 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

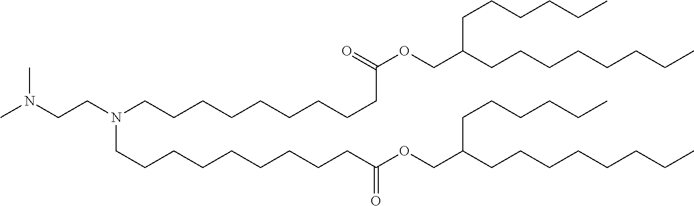 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

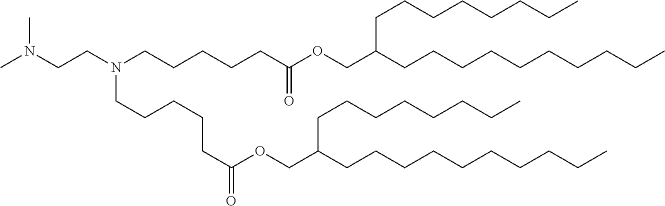 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

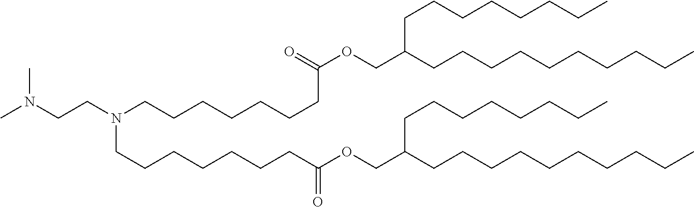 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

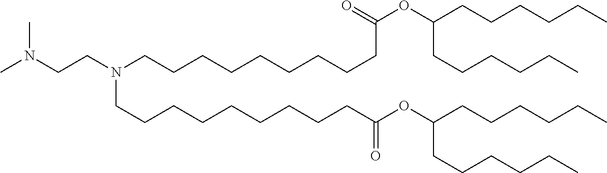 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

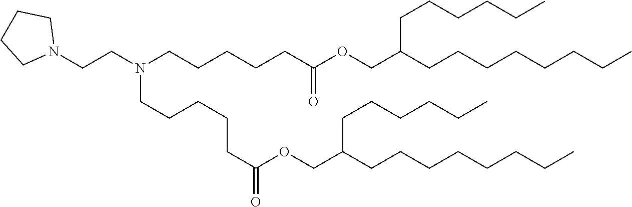 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

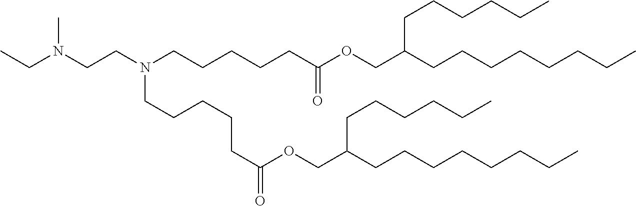 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

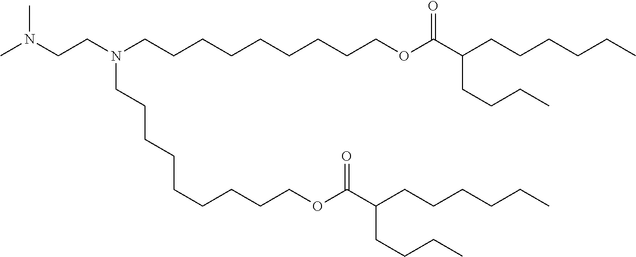 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

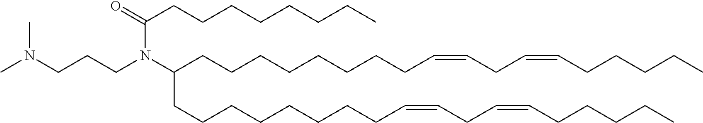 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

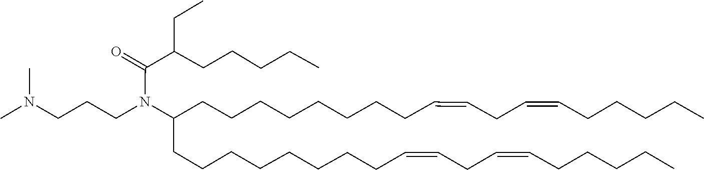 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

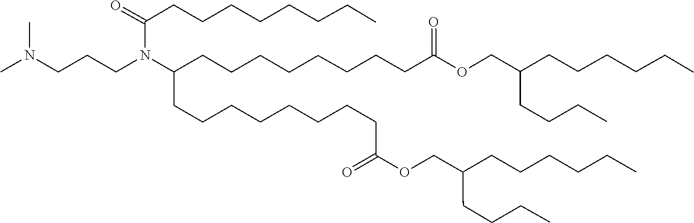 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

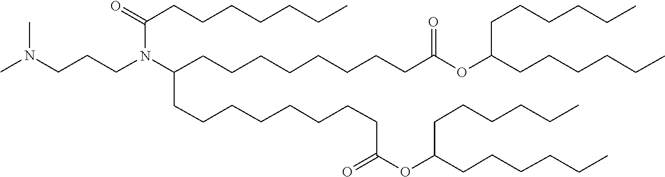 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

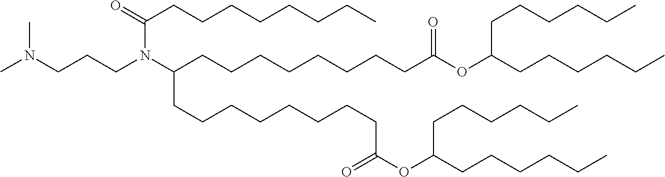 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

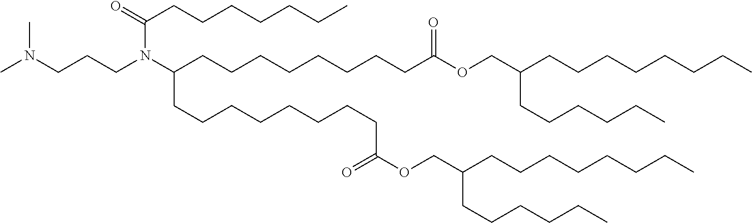 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

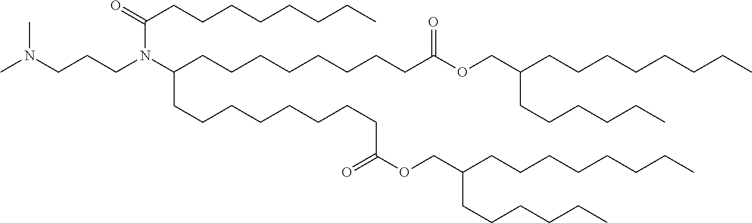 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

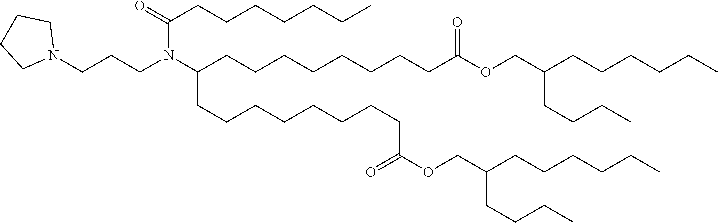 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

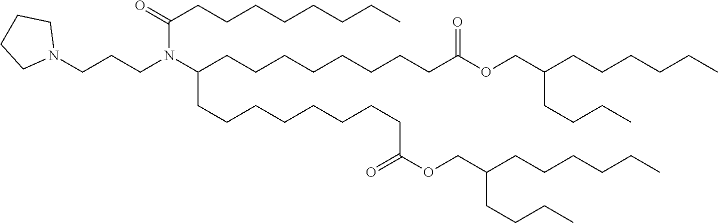 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

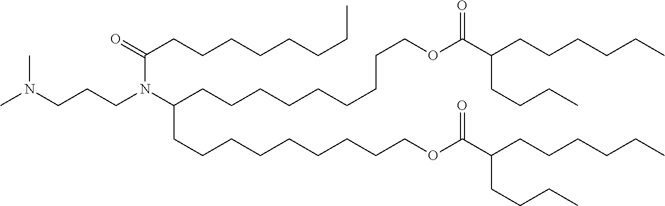 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

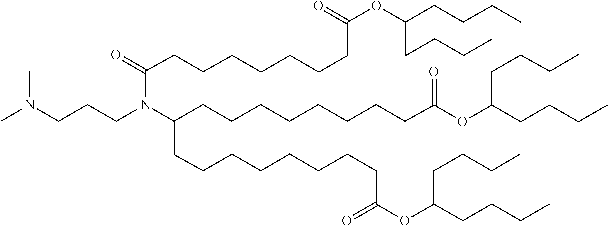 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

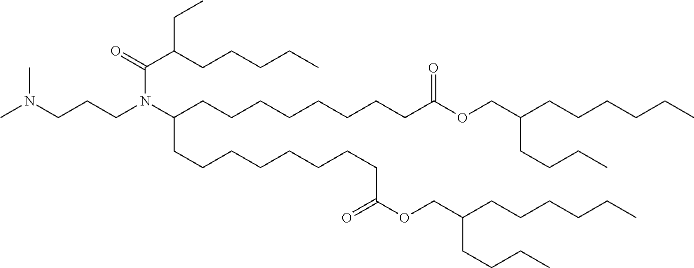 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

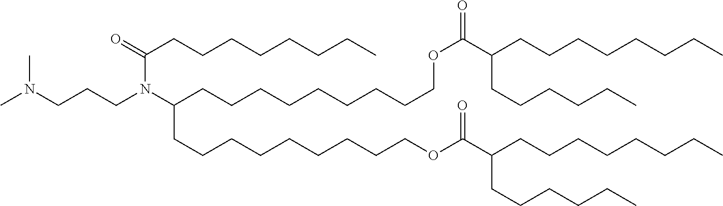 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

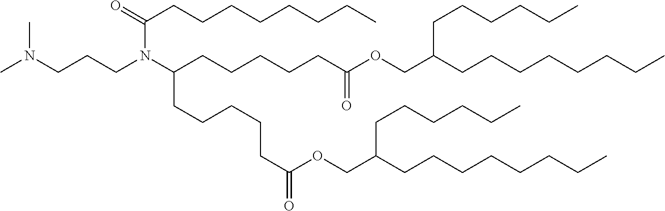 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

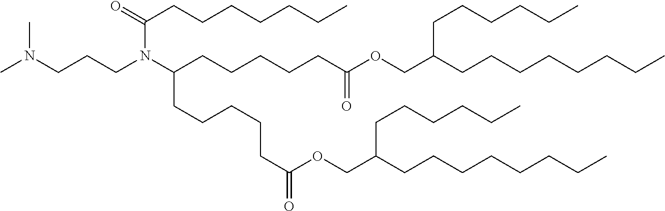 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

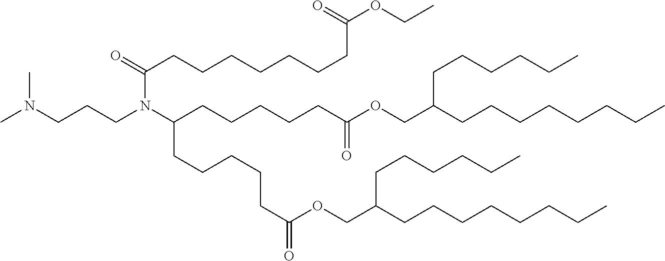 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

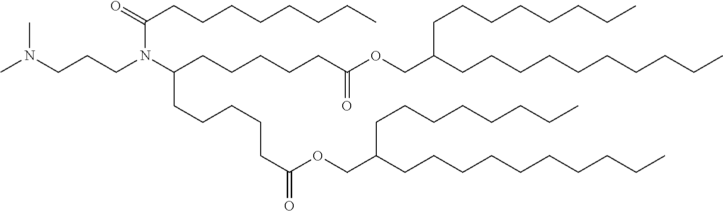 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

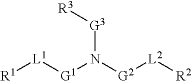

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; Ra is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

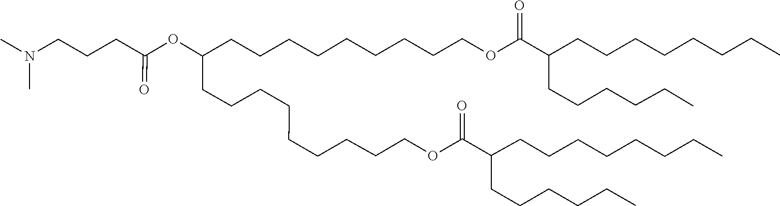 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

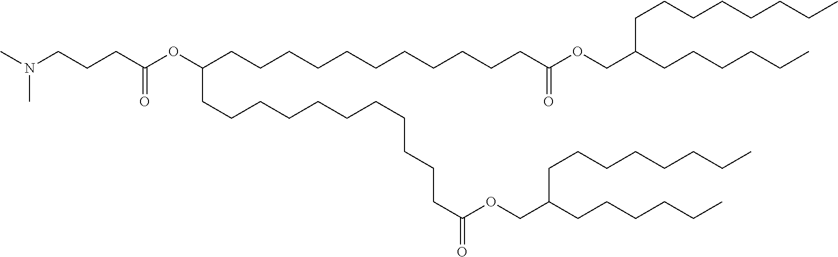 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

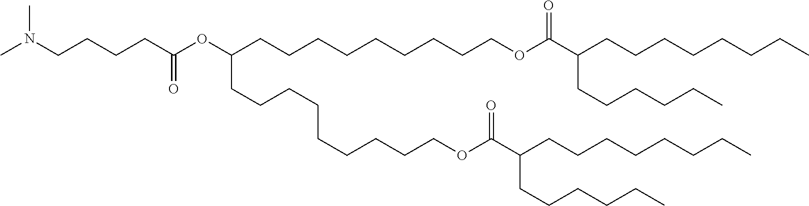 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

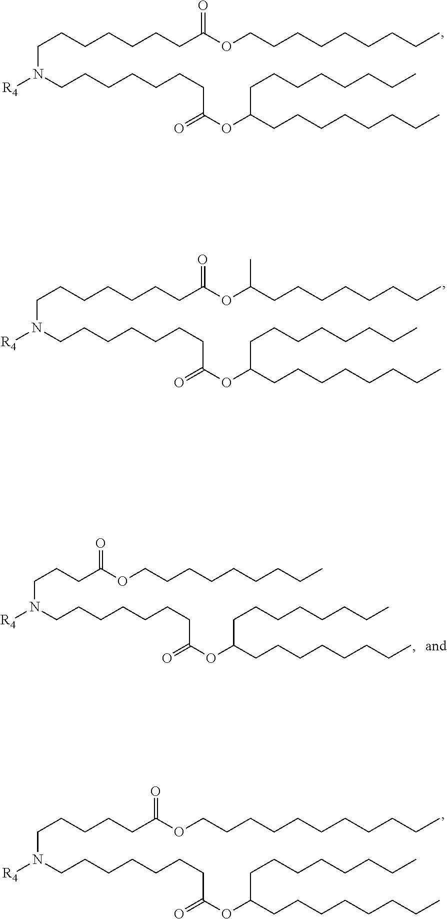

and pharmaceutically acceptable salts thereof. For any one of these four formulas, R⁴ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

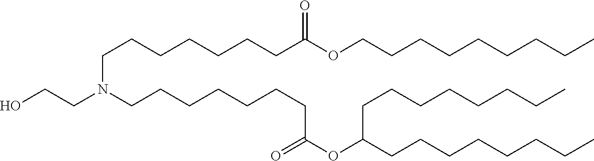 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

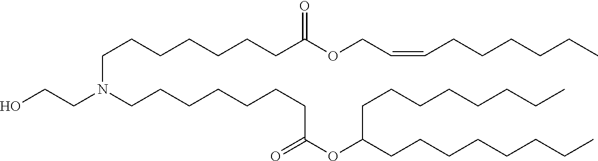 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

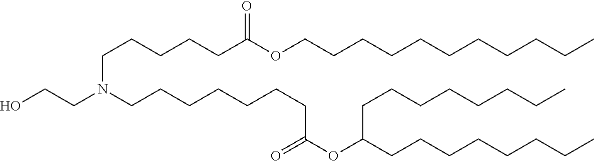 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

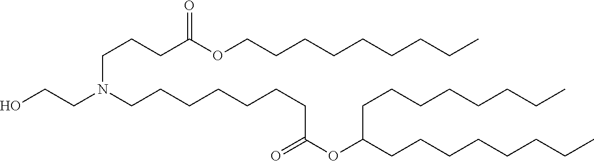 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

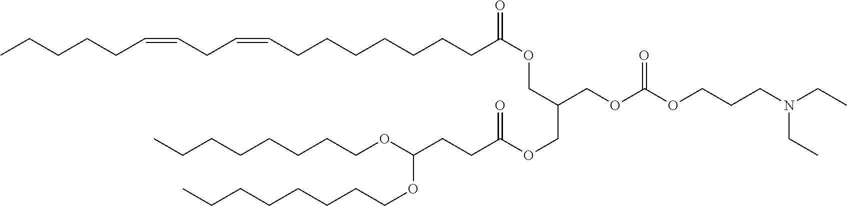 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

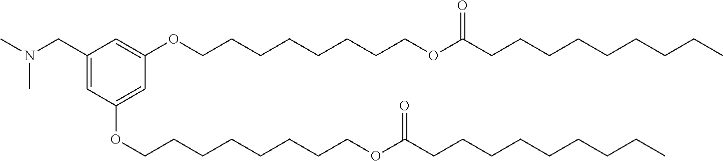 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

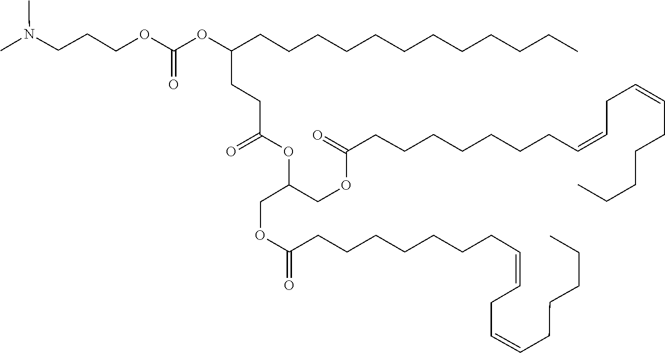 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

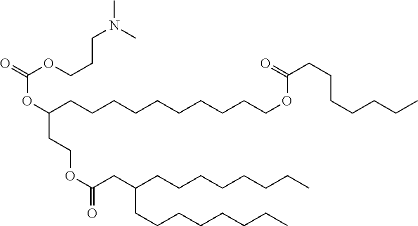 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

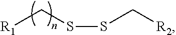

wherein R¹ is selected from the group consisting of imidazole, guanidinium, amino, imine, enamine, an optionally-substituted alkyl amino (e.g., an alkyl amino such as dimethylamino) and pyridyl; wherein R² is selected from the group consisting of one of the following two formulas:

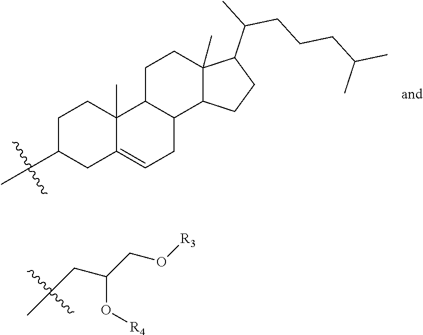

and wherein R³ and R⁴ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

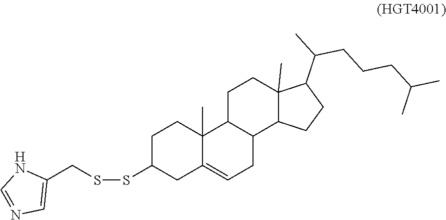 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002” (also referred to herein as “Guan-SS-Chol”), having a compound structure of:

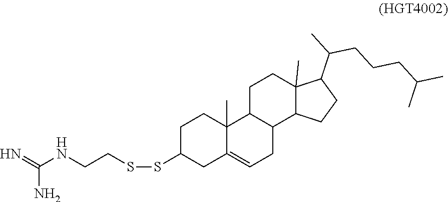 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003”, having a compound structure of:

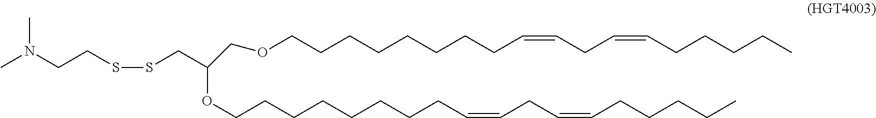 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004”, having a compound structure of:

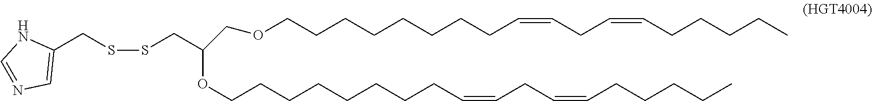 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005”, having a compound structure of:

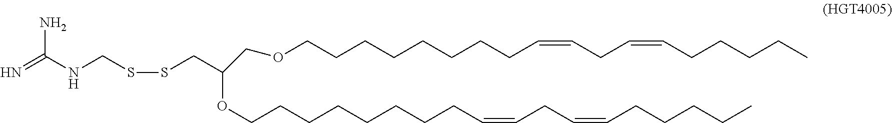 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in U.S. Provisional Application No. 62/672,194, filed May 16, 2018, and incorporated herein by reference. In certain embodiments, the compositions and methods of the present invention include a cationic lipid that is any of general formulas or any of structures (1a)-(21a) and (1b)-(21b) and (22)-(237) described in U.S. Provisional Application No. 62/672,194. In certain embodiments, the compositions and methods of the present invention include a cationic lipid that has a structure according to Formula (I′),

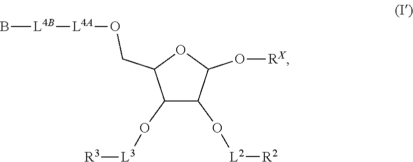

wherein:
   R^{X} is independently —H, or -L^{5A}-L^{5B}-B′;
   each of L¹, L², and L³ is independently a covalent bond, —C(O)—, —C(O)O—, —C(O)S—, or —C(O)NR^{L}—;
   each L^{4A} and L^{5A} is independently —C(O)—, —C(O)O—, or —C(O)NR^{L}—;
   each L⁴B and L^{5B} is independently C₁-C₂₀ alkylene; C₂-C₂₀ alkenylene; or C₂-C₂₀ alkynylene;
   each B and B′ is NR⁴R⁵ or a 5- to 10-membered nitrogen-containing heteroaryl;
   each R′, R², and R³ is independently C₆-C₃₀ alkyl, C₆-C₃₀ alkenyl, or C₆-C₃₀ alkynyl;
   each R⁴ and R⁵ is independently hydrogen, C₁-C₁₀ alkyl; C₂-C₁₀ alkenyl; or C₂-C₁₀ alkynyl; and
   each R^{L} is independently hydrogen, C₁-C₂₀ alkyl, C₂-C₂₀ alkenyl, or C₂-C₂₀ alkynyl.

In certain embodiments, the compositions and methods of the present invention include a cationic lipid that is Compound (139) of 62/672,194, having a compound structure of:

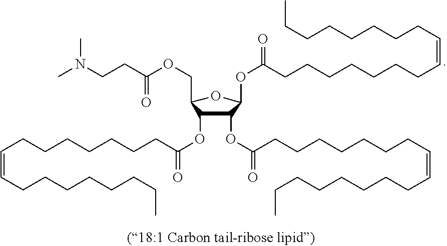

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N42(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-48-[(3P)-cholest-5-en-3-yloxy]octyl)oxy)-N, N-dimethyl-3-[(9Z, 12Z)-octadeca-9, 12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,1 2-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety. In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, one or more cationic lipids suitable for the compositions and methods of the present invention include a cationic lipid that is TL1-04D-DMA, having a compound structure of:

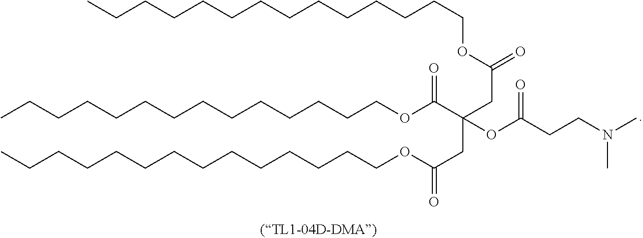 [SMILES: CCCCCCCCCCCCCCOC(=O)CC(CC(=O)OCCCCCCCCCCCCCC)(OC(=O)CCN(C)C)C(=O)OCCCCCCCCCCCCCC]

In some embodiments, one or more cationic lipids suitable for the compositions and methods of the present invention include a cationic lipid that is GL-TES-SA-DME-E18-2, having a compound structure of:

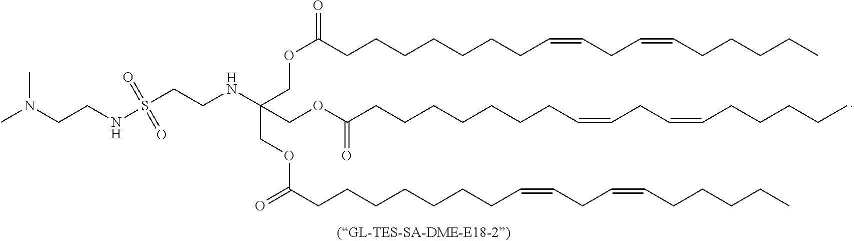 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

In some embodiments, one or more cationic lipids suitable for the compositions and methods of the present invention include a cationic lipid that is SY-3-E14-DMAPr, having a compound structure of:

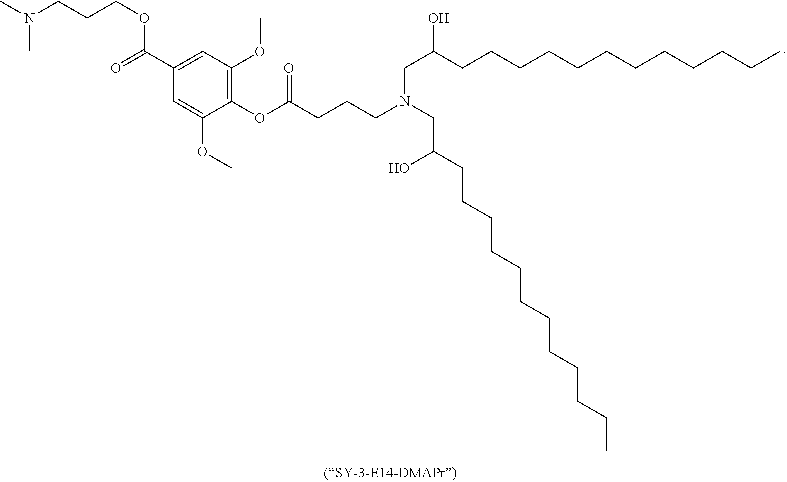 [SMILES: CCCCCCCCCCCCC(O)CN(CCCC(=O)Oc1c(OC)cc(C(=O)OCCCN(C)C)cc1OC)CC(O)CCCCCCCCCCCC]

In some embodiments, one or more cationic lipids suitable for the compositions and methods of the present invention include a cationic lipid that is TL1-01D-DMA, having a compound structure of:

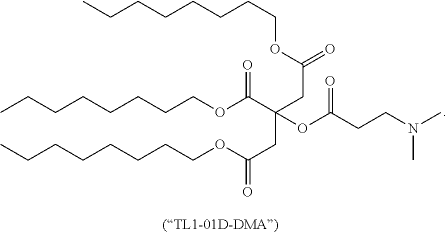 [SMILES: CCCCCCCCOC(=O)CC(CC(=O)OCCCCCCCC)(OC(=O)CCN(C)C)C(=O)OCCCCCCCC]

In some embodiments, one or more cationic lipids suitable for the compositions and methods of the present invention include a cationic lipid that is TL1-10D-DMA, having a compound structure of:

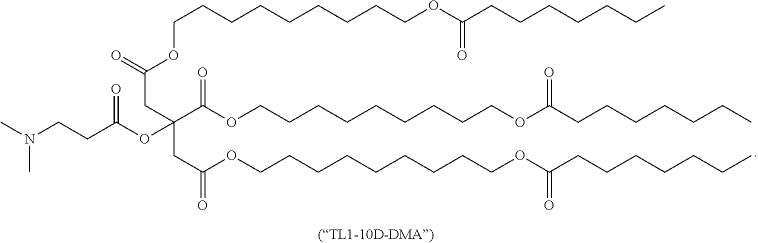 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

In some embodiments, one or more cationic lipids suitable for the compositions and methods of the present invention include a cationic lipid that is GL-TES-SA-DMP-E18-2, having a compound structure of:

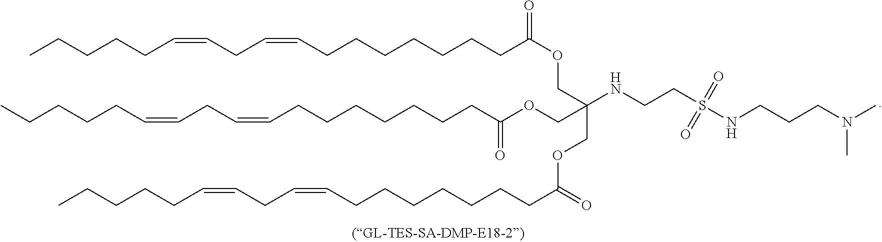 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

In some embodiments, one or more cationic lipids suitable for the compositions and methods of the present invention include a cationic lipid that is REP-E4-E10, having a compound structure of:

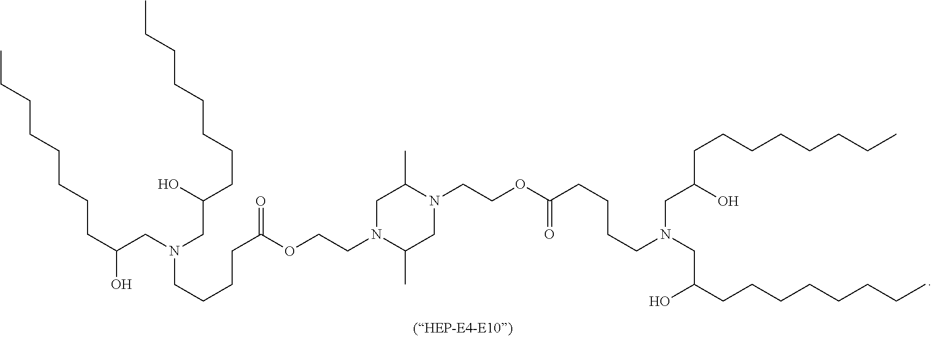 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

In some embodiments, one or more cationic lipids suitable for the compositions and methods of the present invention include a cationic lipid that is REP-E3-E10, having a compound structure of:

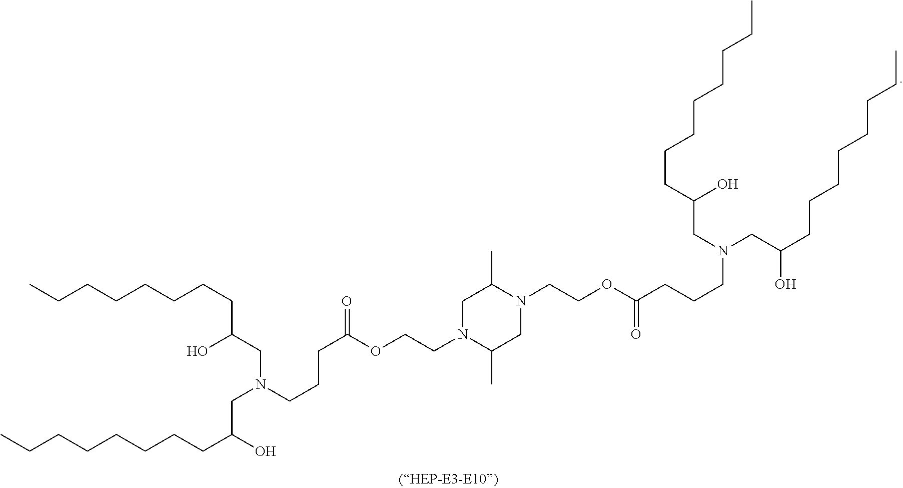 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

Non-Cationic/Helper Lipids

In some embodiments, provided liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

Cholesterol-Based Lipids

In some embodiments, provided liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

PEG-Modified Lipids

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the MCNA to be delivered. Additional considerations include, for example, the saturation of the alkyl chain, as well as the size, charge, pH, pKa, fusogenicity and toxicity of the selected lipid(s). Thus the molar ratios may be adjusted accordingly.

Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

Liposomes Suitable for Use with the Present Invention

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C₁₂-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C₁₂-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C₁₂-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In particular embodiments, a liposome for use with this invention comprises a lipid component consisting of a cationic lipid, a non-cationic lipid (e.g., DOPE or DEPE), a PEG-modified lipid (e.g., DMG-PEG2K), and optionally cholesterol. Cationic lipids particularly suitable for inclusion in such a liposome include GL-TES-SA-DME-E18-2, TL1-01D-DMA, SY-3-E14-DMAPr, TL1-10D-DMA, HGT4002 (also referred to herein as Guan-SS-Chol), GL-TES-SA-DMP-E18-2, HEP-E4-E10, HEP-E3-E10, and TL1-04D-DMA. These cationic lipids have been found to be particularly suitable for use in liposomes that are administered through pulmonary delivery via nebulization. Amongst these, HEP-E4-E10, HEP-E3-E10, GL-TES-SA-DME-E18-2, GL-TES-SA-DMP-E18-2, TL1-01D-DMA and TL1-04D-DMA performed particularly well.

Exemplary liposomes include one of GL-TES-SA-DME-E18-2, TL1-01D-DMA, SY-3-E14-DMAPr, TL1-10D-DMA, GL-TES-SA-DMP-E18-2, REP-E4-E10, REP-E3-E10 and TL1-04D-DMA as a cationic lipid component, DOPE as a non-cationic lipid component, cholesterol as a helper lipid component, and DMG-PEG2K as a PEG-modified lipid component. In some embodiments, the molar ratio of the cationic lipid to non-cationic lipid to cholesterol to PEG-modified lipid may be between about 30-60:25-35:20-30:1-15, respectively. In some embodiments, the molar ratio of cationic lipid to non-cationic lipid to cholesterol to PEG-modified lipid is approximately 40:30:20:10, respectively. In some embodiments, the molar ratio of cationic lipid to non-cationic lipid to cholesterol to PEG-modified lipid is approximately 40:30:25:5, respectively. In some embodiments, the molar ratio of cationic lipid to non-cationic lipid to cholesterol to PEG-modified lipid is approximately 40:32:25:3, respectively. In some embodiments, the molar ratio of cationic lipid to non-cationic lipid to cholesterol to PEG-modified lipid is approximately 50:25:20:5.

In some embodiments, the lipid component of a liposome particularly suitable for pulmonary delivery consists of HGT4002 (also referred to herein as Guan-SS-Chol), DOPE and DMG-PEG2K. In some embodiments, the molar ratio of cationic lipid to non-cationic lipid to PEG-modified lipid is approximately 60:35:5.

Ratio of Distinct Lipid Components

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

(y+z)=100−x.

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. The liposomes for use in provided compositions can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA through electrostatic interactions. For example, during preparation of the compositions of the present invention, cationic liposomes may associate with the mRNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired mRNA into a liposome is often referred to as “loading”. Exemplary methods are described in Lasic, et al., FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of mRNA to the target cell or tissue.

Suitable liposomes in accordance with the present invention may be made in various sizes. In some embodiments, provided liposomes may be made smaller than previously known mRNA encapsulating liposomes. In some embodiments, decreased size of liposomes is associated with more efficient delivery of mRNA. Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-150 (1981), incorporated herein by reference. Average liposome diameter may be reduced by sonication of formed liposomes. Intermittent sonication cycles may be alternated with QELS assessment to guide efficient liposome synthesis.

### Therapeutic Use of Compositions

In one aspect, the present invention, among other things, provides a LNP formulations that encapsulate mRNA that is useful for therapeutic purposes. For example, in some embodiments, the LNP encapsulated mRNA encodes an antibody for the treatment of disease in a subject, such as an immune disease.

In some embodiments, the mRNA is codon optimized. Various codon-optimized methods are known in the art.

### Nebulization

The efficacy of nebulizing a pharmaceutical composition for pulmonary delivery depends on the size of the small aerosol droplets. Generally, the smaller the droplet size, the greater its chance of penetration into and retention in the lung. Large droplets (>10 μm in diameter) are most likely to deposit in the mouth and throat, medium droplets (5-10 μm in diameter) are most likely to deposit between the mouth and airway, and small droplets (<5 μm in diameter) are most likely to deposit and be retained in the lung.

Inhaled aerosol droplets of a particle size of 1-5 μm can penetrate into the narrow branches of the lower airways. Aerosol droplets with a larger diameter are typically absorbed by the epithelia cells lining the oral cavity, and are unlikely to reach the lower airway epithelium and the deep alveolar lung tissue.

Particle size in an aerosol is commonly described in reference to the Mass Median Aerodynamic Diameter (MMAD). MMAD, together with the geometric standard deviation (GSD), describes the particle size distribution of any aerosol statistically, based on the weight and size of the particles. Means of calculating the MMAD of an aerosol are well known in the art.

A specific method of calculating the MMAD using a cascade impactor was first described in 1959 by Mitchell et al. The cascade impactor for measuring particle sizes is constructed of a succession of jets, each followed by an impaction slide, and is based on the principle that particles in a moving air stream impact on a slide placed in their path, if their momentum is sufficient to overcome the drag exerted by the air stream as it moves around the slide. As each jet is smaller than the preceding one, the velocity of the air stream and therefore that of the dispersed particles are increased as the aerosol advances through the impactor. Consequently, smaller particles eventually acquire enough momentum to impact on a slide, and a complete particle size classification of the aerosol is achieved. The improved Next Generation Impactor, used herein to measure the MMAD of the pharmaceutical composition of the invention, was first described by Marple et al. in 2003 and has been widely used in the pharmacopoeia since.

Another parameter to describe particle size in an aerosol is the Volume Median Diameter (VIVID). VIVID also describes the particle size distribution of an aerosol based on the volume of the particles. Means of calculating the VIVID of an aerosol are well known in the art. A specific method used for determining the VIVID is laser diffraction, which is used herein to measure the VIVID of the pharmaceutical composition of the invention (see, e.g., Clark, 1995, Int J Pharm. 115:69-78).

In some embodiments, the mean particle size of the nebulized pharmaceutical composition is between about 4 μm and 6 μm, e.g., about 4 μm, about 4.5 μm, about 5 μm, about 5.5 μm, or about 6 μm.

The Fine Particle Fraction (FPF) is defined as the proportion of particles in an aerosol which have an MMAD or a VIVID smaller than a specified value. In some embodiments, the FPF of the nebulized pharmaceutical composition with a particle size <5 μm is at least about 30%, more typically at least about 40%, e.g., at least about 50%, more typically at least about 60%.

In some embodiments, nebulization is performed in such a manner that the mean respirable emitted dose (i.e., the percentage of FPF with a particle size <5 μm; e.g., as determined by next generation impactor with 15 L/min extraction) is at least about 30% of the emitted dose, e.g., at least about 31%, at least about 32%, at least about 33%, at least about 34%, or at least about 35% the emitted dose. In some embodiments, nebulization is performed in such a manner that the mean respirable delivered dose (i.e., the percentage of FPF with a particle size <5 μm; e.g., as determined by next generation impactor with 15 L/min extraction) is at least about 15% of the emitted dose, e.g., at least 16% or 16.5% of the emitted dose.

### Nebulizer

Nebulization can be achieved by any nebulizer known in the art. A nebulizer transforms a liquid to a mist so that it can be inhaled more easily into the lungs. Nebulizers are effective for infants, children and adults. Nebulizers are able to nebulize large doses of inhaled medications. Typically, a nebulizer for use with the invention comprises a mouthpiece that is detachable. This is important because only clean mouthpieces that are RNase free should be used when administering the pharmaceutical composition of the invention.

In some embodiments, the reservoir volume of the nebulizer ranges from about 5.0 mL to about 8.0 mL. In some embodiments, the reservoir volume of the nebulizer is about 5.0 mL. In some embodiments, the reservoir volume of the nebulizer is about 6.0 mL. In some embodiments, the reservoir volume of the nebulizer is about 7.0 mL. In some embodiments, the reservoir volume of the nebulizer is about 8.0 mL.

One type of nebulizer is a jet nebulizer, which comprises tubing connected to a compressor, which causes compressed air or oxygen to flow at a high velocity through a liquid medicine to turn it into an aerosol, which is then inhaled by the patient.

Another type of nebulizer is the ultrasonic wave nebulizer, which comprises an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Exemplary ultrasonic wave nebulizers are the Omron NE-U17 and the Beurer Nebulizer IH30.

A third type of nebulizer is a mesh nebulizer such as a vibrating mesh nebulizer comprising vibrating mesh technology (VMT). A VMT nebulizer typically comprises a mesh/membrane with 1000-7000 holes that vibrates at the top of a liquid reservoir and thereby pressures out a mist of very fine aerosol droplets through the holes in the mesh/membrane. VMT nebulizers suitable for delivery of the pharmaceutical composition of the invention include any of the following: eFlow (PART Medical Ltd.), i-Neb (Respironics Respiratory Drug Delivery Ltd), Nebulizer IH50 (Beurer Ltd.), AeroNeb Go (Aerogen Ltd.), InnoSpire Go (Respironics Respiratory Drug Delivery Ltd), Mesh Nebulizer (Shenzhen Homed Medical Device Co, Ltd.), Portable Nebulizer (Microbase Technology Corporation) and Airworks (Convexity Scientific LLC). In some embodiments, the mesh or membrane of the VMT nebulizer is made to vibrate by a piezoelectric element. In some embodiments, the mesh or membrane of the VMT nebulizer is made to vibrate by ultrasound.

VMT nebulizers have been found to be particularly suitable for practicing the invention because they do not affect the integrity of the oligonucleotide in the pharmaceutical composition of the invention. Typically, at least about 50%, e.g., at least about 55%, at least about 60%, at least about 65%, at least about 70%, least about 80%, least about 90%, or least about 95% of the oligonucleotide in the pharmaceutical composition of the invention maintains its integrity after nebulization.

In some embodiments, nebulization is continuous during inhalation and exhalation. More typically, nebulization is breath-actuated. Suitable nebulizers for use with the invention have nebulization rate of >0.2 mL/min. In some embodiments, the nebulization rate is >0.25 mL/min. In other embodiment, the nebulization rate is >0.3 mL/min. In some embodiments, the nebulization rate is >0.45 mL/min. In a typical embodiment, the nebulization rate ranges between 0.2 mL/minute and 0.5 mL/minute.

A human subject may display adverse effects during treatment, when the nebulization volume exceeds 10 mL. In particular, such adverse effects may be more common when volumes greater than 20 mL are administered. In some embodiments, the nebulization volume does not exceed 20 mL.

In some embodiments, a single dose of the pharmaceutical composition of the invention can be administered with only a one or two refills per nebulization treatment. For example, if the total volume of the pharmaceutical composition that is to be administered to the patient is 13 mL, then only a single refill is required to administer the entire volume when using a nebulizer with an 8 mL reservoir, but two refills are required to administer the same volume when using a nebulizer with a 5 mL reservoir. In another embodiment, at least three refills are required per nebulization treatment, e.g., to administer a volume of 26 mL, at least three refills are required when using a nebulizer with an 8 mL reservoir. In yet a further embodiment, at least four refills are required. For example, to deliver 42 mL with a nebulizer having a 5 mL reservoir, at least eight refills are required. Typically, no more than 1-3 refills will be required to administer the pharmaceutical composition of the invention.

The pharmaceutical composition of the invention is typically nebulized at a rate ranging from 0.2 mL/minute to 0.5 mL/minute. A concentration of 0.5 mg/ml to 0.8 mg/ml of the oligonucleotide (e.g. about 0.6 mg/ml) has been found to be particularly suitable, in particular when administered with a vibrating mesh nebulizer.

In some embodiments, the number of nebulizers used during a single nebulization session ranges from 2-8. In some embodiments, the number of nebulizers used during a single nebulization session ranges from 1-8. In some embodiments, 1 nebulizer is used during a single nebulization session. In some embodiments, 2 nebulizers are used during a single nebulization session. In some embodiments, 3 nebulizers are used during a single nebulization session. In some embodiments, 4 nebulizers are used during a single nebulization session. In some embodiments, 5 nebulizers are used during a single nebulization session. In some embodiments, 6 nebulizers are used during a single nebulization session. In some embodiments, 7 nebulizers are used during a single nebulization session. In some embodiments, 8 nebulizers are used during a single nebulization session.

### EXAMPLES

While certain compounds, compositions and methods of the present invention have been described with specificity in accordance with certain embodiments, the following examples serve only to illustrate the compounds of the invention and are not intended to limit the same.

### Example 1. Encapsulation of mRNA-Encoding Antibodies in Lipid Nanoparticles (LNPs)

This example illustrates encapsulation of mRNA-encoded antibodies in lipid nanoparticles (LNPs) and preparation of LNP formulations comprising mRNA encoding antibodies.

Exemplary LNP formulations were prepared by a conventional process of encapsulating oligonucleotides by mixing oligonucleotides with a mixture of lipids, without first pre-forming the lipids into lipid nanoparticles, as described in US 2016/0038432 (Process A).

An exemplary mRNA-encoding anti-IL6R LNP formulation was prepared comprising 10% trehalose (w/v) and an exemplary LNP composition of DMG-PEG 2000 (PEG): Guan-SS-chol (cationic lipid): cholesterol: DOPE (helper lipid) of 5:60:0:35. A high encapsulation efficiency of 99.36% was achieved. Particle size of about 58 nm was obtained with a polydispersity index of 0.16.

Another exemplary mRNA-encoding anti-IL4Rα LNP formulation was prepared with an exemplary LNP composition of DMG-PEG 2000 (PEG): Guan-SS-chol (cationic lipid): cholesterol: DOPE (helper lipid) of 5:60:0:35, and similarly, a high encapsulation efficiency of 98% was achieved. Particle size of about 60 nm was obtained with a polydispersity index of 0.18. The LNP formulations are described in Table 3 below.

TABLE 3
LNP Formulations comprising Messenger RNA encoding Antibodies

| | | | | | | | Particle | | |
|---|---|---|---|---|---|---|---|---|---|
| Test | | | Cationic | Helper | | Composition | size | | Encapsulation |
| Substance | N/P | Buffer | lipid | lipid | PEG | (PEG:cat:chol:help:add) | (nm) | PDI | % |
| Saline | NA | NA | NA | NA | NA | 0:0:0:0:0 | | | |
| Anti- | 4 | 10% | Guan- | DOPE | DMG- | 5:60:0:35 | 58 | 0.16 | 99.36 |
| IL6R | | trehalose | SS-chol | | PEG_2000 | | | | |
| Anti- | 4 | 10% | Guan- | DOPE | DMG- | 5:60:0:35 | 60 | 0.18 | 98.0 |
| IL4Rα | | trehalose | SS-chol | | PEG_2000 | | | | |

This example demonstrated that lipid nanoparticles with high encapsulation efficiencies were achieved comprising mRNA encoding antibodies like anti-IL6R and anti-IL4Rα.

### Example 2. Pharmacodynamic Study of mRNA-Encoded Antibodies in Mice

This example illustrates a pharmacodynamic study in mice that were administered mRNA-encoded antibodies. Antibody levels were subsequently evaluated in mice that were administered mRNA-encoded antibodies relative to control mice that were administered saline. The study design is outlined in Table 4 below.

TABLE 4
Study Design in Mice

| Test | No. of | Dose Levels | Concentration | Dose Volume |
|---|---|---|---|---|
| substance | animals | (μg/animal) | (mg/ml) | (μL/animal) |
| Saline | 4 | NA | NA | 50 |
| Anti-IL6R | 8 | 15 | 0.3 | 50 |
| Anti-IL4Rα | 8 | 15 | 0.3 | 50 |

Messenger RNA encoding antibodies encapsulated in LNPs directed to various immune targets were administered to mice. Exemplary targets include IL-4, IL-5, IL-6, IL-9, IL-13, IL-25, IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1) and other drivers of Type 2 inflammation, as well as additional targets. In this example, mRNA-LNPs encapsulating anti-IL6R and anti-IL4Rα were tested.

CD1 mice that were about 8-10 weeks old were administered mRNA-LNPs encapsulating anti-IL6R or anti-IL4Rα at a dose of about 15 micrograms/animal by intratracheal administration. Control mice were administered saline. At day 4, blood was collected for serum preparation. Mice were sacrificed and tissue samples including Bronchoalveolar Lavage Fluid (BALF), lung and liver were collected. BALF samples were collected by five sequential plunges of fluid. The first BALF fraction collected is the cytokine-rich BALF and the BAL cell pellet. Whole left and right lungs and two 8 mm biopsy punches from the liver were snap frozen in liquid nitrogen.

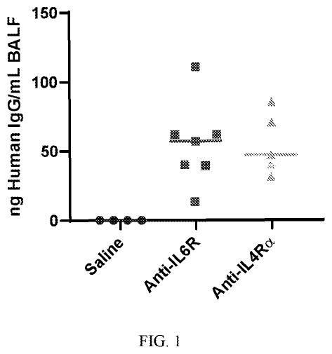

The levels of human IgG were measured 72 hours after administration of mRNA therapy in BALF from mice treated with anti-IL6R or anti-IL4Rα antibodies relative to mice that received a saline control. Antibody levels were quantified and the data are shown in FIG. 1 and Table 5.

TABLE 5
Antibody Levels from mRNA LNPs in BALF

| | | Average Antibody |
|---|---|---|
| | Antibody levels | levels in BALF |
| | in BALF | (±Standard Deviation) |
| Test | (ng/mL) | (ng/mL) |
| Saline | ND | ND |
| | ND | ND |
| | ND | ND |
| | ND | ND |
| Anti-IL6R | 40.535 | 55.17271 |
| | 61.761 | (±29.80997) |
| | 110.925 | |
| | 57.079 | |
| | 14.083 | |
| | — | |
| | 61.811 | |
| | 40.015 | |
| Anti-IL4Rα | 70.311 | 55.2664 |
| | — | (±21.8746) |
| | 40.879 | |
| | 85.114 | |
| | 32.124 | |
| | — | |
| | 47.904 | |
| | — | |

This example demonstrated that mRNA encoding antibodies were delivered and human antibodies were expressed in target lung tissues. All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of treating an immune disease in a subject, the method comprising: administering to a subject in need thereof one or more mRNAs encoding a heavy chain and a light chain of an antibody that binds to a protein target selected from IL-4, IL-5, IL-6, IL-9, IL-13, IL-25, or IL-33, and wherein the one or more mRNAs are encapsulated in a lipid nanoparticle (LNP).

2. A method of treating an immune disease in a subject, the method comprising: administering to a subject in need thereof one or more mRNAs encoding a heavy chain and a light chain of an antibody that binds to a protein target selected from IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, IL1RL1), and wherein the one or more mRNAs are encapsulated in a lipid nanoparticle (LNP).

3. The method of claim 1 or 2, wherein the antibody inhibits a protein target selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP).

4. The method of claim 1 or 2, wherein the antibody is an anti-IL6R antibody or an anti-IL4Rα antibody.

5. The method of claims 1-4, wherein the immune disease is selected from asthma, chronic rhinosinusitis with nasal polyps (CRSwNP), chronic obstructive pulmonary disease (COPD), systemic sclerodis—interstial lung disease (SSc-ILD), idiopathic pulmonary fibrosis IPF, sarcoidosis, or allergy.

6. The method of claims 1-4, wherein the immune disease is selected from atopic dermatitis, asthma with eosinophilic phenotype or with oral corticosteroid-dependent asthma, chronic rhinosinusitis with nasal polyposis, pediatric atopic dermatitis, pediatric asthma, eosphinohilic esophagitis, COPD, prurigo nodularis, chronic spontaneous urticaria and bullous phemphigoid, grass pollen allergy and peanut allergy.

7. The method of claims 1-4, wherein the immune disease is selected from rheumatoid arthritis, polyarticular juvenile idiopathic arthritis, systemic juvenile arthritis, or severe Covid-19 disease.

8. The method of any one of the preceding claims, wherein the administering is performed by nebulization, intratracheal delivery, or inhalation.

9. The method of any one of the preceding claims, wherein the administering results in administration of the mRNA to lung tissue.

10. The method of claim 9, wherein the administering results in antibody expression for at least about 48 hours, 72 hours, 96 hours, or 120 hours.

11. The method of claim 9, wherein low or no systemic exposure of the mRNA occurs.

12. The method of claims 1-4, wherein the immune disease is selected from autoimmune dermatitis or atopic dermatitis.

13. The method of claims 1-12, wherein the administering is performed intravenously.

14. The method of claim 1-12, wherein the administering is performed intraperitoneally.

15. The method of claim 13 or 14, wherein the antibody is expressed systemically.

16. The method of any one of the preceding claims, wherein the LNP comprises one or more of cationic lipid, non-cationic lipid, and PEG-modified lipids.

17. The method of claim 16, wherein the LNP further comprises cholesterol.

18. The method of claim 16, wherein the LNP has a molar ratio of cationic lipid(s) to non-cationic lipid(s) to PEG-modified lipid(s) between about 30-60:25-35: 1-15, respectively.

19. The method of any one of claims 16-18, wherein the non-cationic lipid is selected from 1,2-Dierucoyl-sn-glycero-3-phosphoethanolamine (DEPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

20. The method of any one of the preceding claims, wherein the LNP comprises DMG-PEG-2000, Guan-SS-Chol, and DOPE.

21. The method of claim 20, where in the DMG-PEG-2000, Guan-SS-Chol, and DOPE are present at a ratio of about 5:60:35.

22. The method of any one of the preceding claims, wherein the heavy chain and the light chain are encoded in single mRNA.

23. The method of any one of claims 1-21, wherein the heavy chain and the light chain are encoded in separate mRNAs.

24. The method of any one of the preceding claims, wherein the LNP has a size of no greater than 150 nm.

25. The method of any one of claims 1-23, wherein the LNP has a size of no greater than 100 nm.

26. The method of any one of claims 1-23, wherein the LNP has a size of no greater than 75 nm.

27. The method of any one of the preceding claims, wherein the LNP has a size of about 60 nm.

28. The method of any one of the preceding claims, wherein the one or more mRNAs are modified to enhance stability.

29. The method of claim 26, wherein the one or more mRNAs are modified to include a modified nucleotide, a cap structure, a poly A tail, a 5′ and/or 3′ untranslated region.

30. The method of any one of claims 1-29, wherein the one or more mRNAs are unmodified.

31. A composition comprising one or more mRNAs encoding a heavy chain and a light chain of an antibody that binds to a protein target selected from IL-4, IL-5, IL-6, IL-9, IL-13, IL-25, or IL-33, and wherein the one or more mRNAs are encapsulated in a lipid nanoparticle (LNP).

32. A composition comprising one or more mRNAs encoding a heavy chain and a light chain of an antibody that binds to a protein target selected from IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, IL1RL1), and wherein the one or more mRNAs are encapsulated in a lipid nanoparticle (LNP).

33. The composition of claim 31 or 32, wherein the antibody inhibits a protein target selected from IL-4, IL-5, IL-6, IL-9, IL-13, IL-25, or IL-33, IL-4 Receptor (IL-4R, e.g., IL-4Rα), IL-5 Receptor (IL-5R), IL-6 Receptor (IL-6R), IL-9 Receptor (IL-9R), IL-13 Receptor (IL-13R), IL-25 Receptor (IL-25R) or IL-33 Receptor (IL-33R, e.g. ST2, also known as IL1RL1), and wherein the one or more mRNAs are encapsulated in a lipid nanoparticle (LNP).

34. The composition of claim 31-33, wherein the antibody is an anti-IL6R antibody or an anti-IL4Rα antibody.

35. The composition of any one of claims 31-34, wherein the LNP comprises one or more of cationic lipid, non-cationic lipid, and PEG-modified lipids.

36. The composition of claim 35, wherein the LNP further comprises cholesterol.

37. The composition of claim 36, wherein the LNP has a molar ratio of cationic lipid(s) to non-cationic lipid(s) to PEG-modified lipid(s) between about 30-60:25-35:1-15, respectively.

38. The composition of claims 35-37, wherein the non-cationic lipid is selected from 1,2-Dierucoyl-sn-glycero-3-phosphoethanolamine (DEPE), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, or 1-stearoyl-2-oleoyl-phosphatidyethanolamine (SOPE).

39. The composition of any one of the claims 31-38, wherein the LNP comprises DMG-PEG-2000, Guan-SS-Chol, and DOPE.

40. The composition of claim 39, where in the DMG-PEG-2000, Guan-SS-Chol, and DOPE are present at a ratio of about 5:60:35.

41. The composition of claim 34, wherein the mRNA encodes an anti-IL6R antibody heavy chain comprising a sequence at least 80% identical to
(SEQ ID NO: 18)
EVQLVESGGGLVQPGRSLRLSCAASRFTFDDYAMHWVRQAPGKGLEWVS
GISWNSGRIGYADSVKGRFTISRDNAENSLFLQMNGLRAEDTALYYCAK
GRDSFDIWGQGTMVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPR
EPQVTYLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPGK.

42. The composition of claim 41, wherein the mRNA encodes an anti-IL6R antibody heavy chain further comprising a secretion sequence at least 80% identical to:
(SEQ ID NO: 26)
MATGSRTSLLLAFGLLCLPWLQEGSAFPTIPLS.

43. The composition of claim 41, wherein the mRNA encodes an anti-IL6R antibody light chain comprising a sequence at least 80% identical to:
(SEQ ID NO: 19)
DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

44. The composition of claim 41, wherein the mRNA encodes an anti-IL6R antibody light chain further comprising a secretion sequence at least 80% identical to:
(SEQ ID NO: 26)
MATGSRTSLLLAFGLLCLPWLQEGSAFPTIPLS.

45. The composition of claim 42, wherein the mRNA that encodes the anti-IL6R antibody heavy chain is codon optimized and comprises a sequence at least 80% identical to one of the following sequences:
(A)
(SEQ ID NO: 2)
ATGGCCACTGGAAGCCGGACAAGCCTGCTGCTGGCCTTTGGCCTGCTGTGTCTGCCTTGGCTGCAGGAGGGAAGCGCATTT
CCAACAATTCCTCTGAGCGAAGTGCAGCTGGTGGAGTCTGGAGGAGGCCTCGTGCAGCCAGGCAGATCCCTGAGGCTCTCC
TGCGCCGCTAGCAGATTCACTTTCGACGACTACGCCATGCACTGGGTGCGGCAGGCACCTGGCAAGGGACTGGAATGGGTG
TCCGGCATTTCTTGGAACAGCGGGCGGATCGGGTACGCGGACAGCGTGAAAGGAAGGTTTACAATCTCCCGGGACAATGCT
GAGAACTCCCTGTTCCTGCAGATGAACGGCCTGAGAGCTGAAGACACAGCACTGTACTATTGCGCAAAAGGCCGCGACTCC
TTTGACATCTGGGGGCAGGGCACAATGGTGACCGTGTCTAGCGCCTCCACAAAAGGACCTAGCGTTTTCCCACTGGCTCCA
TCTAGCAAGTCTACATCCGGGGGCACCGCCGCTCTGGGCTGTCTGGTGAAGGATTACTTCCCTGAGCCCGTCACTGTCAGC
TGGAACTCCGGAGCTCTGACCTCAGGCGTGCACACTTTTCCCGCTGTGCTGCAGAGCTCTGGCCTGTACAGCCTGAGCAGC
GTTGTGACCGTGCCTAGCTCATCCCTCGGCACCCAGACCTATATCTGCAACGTCAACCACAAACCTTCCAACACCAAAGTG
GACAAGAAAGTGGAACCTAAGTCCTGCGATAAGACTCATACTTGCCCTCCTTGTCCAGCACCAGAGCTGCTGGGGGGGCCA
AGCGTGTTTCTCTTTCCACCTAAGCCTAAAGACACCCTGATGATCTCCAGGACCCCAGAGGTGACATGTGTGGTGGTGGAC
GTGTCTCATGAGGACCCTGAGGTGAAATTCAATTGGTATGTGGACGGCGTTGAGGTTCACAACGCAAAGACCAAGCCAAGG
GAGGAGCAGTATAATAGCACCTATCGCGTGGTGTCCGTCCTGACAGTGCTGCACCAGGACTGGCTGAACGGGAAGGAGTAT
AAGTGTAAAGTGAGCAACAAGGCACTGCCTGCTCCTATCGAGAAGACTATCAGCAAAGCTAAAGGACAGCCAAGAGAGCCC
CAGGTGACCTACCTGCCACCTTCTCGGGACGAACTGACCAAAAACCAGGTGAGCCTGACTTGCCTGGTGAAGGGCTTTTAT
CCCTCTGATATTGCAGTGGAGTGGGAGAGTAACGGGCAGCCCGAGAACAACTACAAGACTACTCCACCAGTTCTGGATTCC
GACGGCAGCTTCTTCCTGTATAGCAAACTGACAGTGGACAAGTCCCGGTGGCAGCAGGGCAACGTTTTTAGCTGCAGCGTG
ATGCATGAGGCTCTGCACAACCATTACACACAGAAGTCTCTGTCTCTGTCCCCCGGAAAGTGA;
(B)
(SEQ ID NO: 3)
ATGGCCACCGGGTCTCGGACAAGCCTCCTGCTCGCATTCGGGCTCCTGTGTCTGCCTTGGCTGCAAGAAGGATCCGCATTT
CCCACCATTCCACTGTCTGAGGTGCAGCTGGTCGAGTCTGGAGGAGGACTGGTGCAGCCTGGCAGGTCTCTGAGGCTGTCT
TGCGCTGCCAGCCGGTTTACCTTTGATGATTACGCCATGCACTGGGTGAGGCAGGCTCCCGGCAAAGGACTGGAATGGGTG
TCCGGAATTTCCTGGAATAGTGGCAGGATCGGCTATGCCGACTCTGTCAAAGGCCGGTTTACAATCTCCCGCGACAACGCT
GAGAACTCCCTGTTCCTGCAGATGAACGGCCTGAGAGCTGAAGATACCGCCCTGTACTATTGCGCCAAGGGGCGCGACAGC
TTCGACATTTGGGGCCAGGGAACCATGGTGACTGTGAGCAGCGCATCCACAAAAGGGCCCTCCGTGTTCCCCCTGGCACCT
TCCAGTAAATCCACTTCTGGCGGAACAGCAGCTCTCGGCTGTCTGGTGAAGGATTATTTCCCCGAGCCAGTGACAGTGTCT
TGGAATTCTGGCGCACTCACCAGTGGAGTCCACACTTTTCCAGCCGTGCTGCAGAGCTCCGGACTGTATTCCCTGAGCTCC
GTCGTGACAGTGCCATCCTCTTCTCTGGGAACTCAGACATATATTTGCAACGTTAATCATAAGCCTTCTAACACCAAGGTG
GATAAGAAAGTGGAGCCCAAATCCTGCGACAAGACACACACTTGCCCACCATGCCCTGCCCCTGAACTGCTGGGAGGACCA
AGCGTGTTTCTCTTCCCTCCTAAGCCTAAGGATACCCTGATGATCTCTAGGACCCCAGAGGTGACATGCGTGGTGGTTGAC
GTCTCCCATGAAGATCCTGAAGTGAAATTTAACTGGTACGTGGACGGAGTGGAAGTGCACAATGCAAAGACCAAACCCCGC
GAGGAACAGTACAACTCCACTTACCGGGTGGTTTCCGTGCTGACAGTGCTGCACCAGGATTGGCTGAACGGAAAAGAGTAT
AAGTGCAAAGTGAGCAATAAGGCCCTGCCCGCCCCTATTGAGAAGACCATCTCTAAGGCTAAAGGCCAGCCTCGCGAACCC
CAGGTTACCTATCTGCCTCCAAGCAGAGATGAGCTCACCAAAAACCAGGTGTCTCTGACCTGTCTGGTGAAAGGCTTCTAT
CCAAGCGATATCGCCGTGGAGTGGGAGTCCAACGGACAGCCAGAAAACAATTACAAGACTACCCCACCTGTCCTGGACAGC
GACGGGAGCTTCTTTCTGTACTCTAAGCTGACAGTCGACAAAAGCCGGTGGCAGCAAGGCAACGTCTTCAGCTGCAGCGTC
ATGCACGAGGCCCTGCATAATCATTATACTCAGAAGTCTCTGAGCCTGAGCCCTGGCAAGTAG;
(C)
(SEQ ID NO: 4)
ATGGCCACTGGAAGCAGAACCTCCCTGCTGCTGGCATTCGGACTGCTGTGCCTGCCATGGCTGCAGGAGGGATCCGCTTTC
TCCCAACCATCCCCCTCAGCGAGGTGCAGCTCGTTGAATCTGGAGGAGGACTGGTGCAACCAGGACGCTCCCTGAGACTGT
CTTGTGCTGCTTCCAGGTTTACTTTTGACGATTATGCTATGCACTGGGTGAGACAGGCCCCAGGAAAAGGACTGGAATGGG
TGTCTGGAATTTCTTGGAACAGCGGACGCATTGGCTACGCCGACTCTGTGAAGGGAAGGTTTACTATCTCCAGGGATAACG
CGGAAAACTCCCTCTTCCTCCAGATGAACGGCCTGAGGGCAGAGGACACCGCTCTGTACTACTGCGCCAAAGGAAGAGATA
GCTTCGATATCTGGGGACAGGGGACCATGGTGACAGTTTCCAGCGCTAGCACCAAGGGCCCCAGCGTGTTCCCACTGGCCC
CATCCTCCAAGAGCACTTCTGGCGGGACTGCTGCACTGGGCTGCCTGGTGAAGGATTATTTCCCTGAGCCTGTGACAGTGA
GCTGGAACTCAGGAGCACTGACTTCCGGGGTGCATACATTCCCCGCTGTGCTGCAGTCTTCTGGGCTGTATTCCCTCAGCA
GCGTGGTGACCGTCCCTTCCTCAAGCCTGGGAACCCAGACATATATTTGTAACGTGAACCACAAGCCAAGCAATACAAAGG
TGGATAAGAAGGTGGAGCCTAAGTCCTGTGACAAAACACACACATGTCCCCCATGTCCAGCTCCTGAACTGCTTGGCGGAC
CATCCGTCTTCCTGTTTCCACCCAAACCAAAGGATACACTGATGATCAGCCGGACACCAGAGGTGACTTGCGTCGTCGTGG
ACGTCAGCCATGAAGACCCCGAGGTGAAGTTTAATTGGTATGTGGACGGGGTGGAGGTCCACAACGCCAAGACCAAACCCA
GGGAGGAGCAGTACAACTCCACTTATCGCGTGGTTTCTGTGCTGACAGTCCTGCACCAGGATTGGCTGAACGGGAAGGAGT
ACAAATGCAAAGTGTCCAATAAGGCCCTGCCCGCCCCAATCGAGAAAACTATTTCAAAGGCCAAAGGACAGCCCAGAGAGC
CACAGGTGACCTACCTCCCTCCTTCCAGGGACGAGCTCACTAAGAATCAGGTGTCTCTGACTTGCCTGGTGAAAGGCTTTT
ATCCTTCTGACATCGCAGTGGAGTGGGAGAGCAATGGCCAGCCCGAGAACAATTATAAAACAACACCACCCGTCCTGGACT
CTGATGGCAGCTTTTTCCTGTATAGCAAGCTGACAGTGGACAAATCACGCTGGCAGCAGGGGAATGTCTTCAGCTGTAGCG
TGATGCACGAAGCTCTGCACAATCACTATACACAGAAGTCCCTGTCCCTGAGCCCAGGAAAATAA;
or
(D)
(SEQ ID NO: 5)
ATGGCTACCGGCAGCAGGACTAGCCTGCTGCTGGCTTTCGGCCTGCTGTGTCTGCCTTGGCTGCAAGAGGGGTCCGCTTTC
CCTACTATCCCTCTGTCCGAAGTGCAGCTGGTCGAGAGCGGAGGGGGCCTGGTGCAGCCTGGAAGAAGTCTGCGCCTGTCC
TGCGCAGCAAGCAGGTTTACATTTGACGACTACGCAATGCACTGGGTGCGCCAAGCTCCAGGCAAAGGCTTAGAATGGGTG
TCTGGCATCAGCTGGAACTCAGGGCGGATCGGCTACGCAGACAGCGTGAAGGGCAGGTTCACTATCTCTAGGGACAACGCC
GAGAACTCCCTGTTCCTGCAGATGAATGGGCTGCGGGCAGAAGACACTGCACTGTATTATTGTGCTAAGGGGAGAGACTCT
TTCGACATCTGGGGCCAGGGCACAATGGTGACTGTGTCCTCTGCCTCTACCAAGGGCCCTTCCGTGTTCCCACTGGCACCA
AGCAGCAAATCCACATCCGGGGGGACCGCAGCTCTCGGATGTCTGGTGAAAGACTATTTCCCTGAGCCCGTCACAGTGTCT
TGGAATTCCGGCGCCCTGACAAGCGGCGTGCACACTTTTCCTGCCGTTCTGCAGAGCTCCGGCCTATACTCCCTGTCCAGC
GTGGTGACAGTCCCTTCTAGCAGTCTGGGCACACAGACTTATATTTGCAACGTGAATCACAAGCCATCTAACACCAAGGTG
GATAAGAAGGTGGAACCAAAGTCCTGTGATAAAACCCATACCTGTCCTCCATGTCCAGCTCCTGAACTCCTGGGGGGACCC
TCTGTGTTCCTGTTCCCACCTAAGCCTAAAGACACTCTGATGATTTCCAGAACTCCTGAGGTGACTTGCGTGGTGGTGGAT
GTGTCCCATGAGGATCCTGAGGTCAAGTTCAACTGGTACGTGGACGGAGTCGAGGTGCATAACGCTAAAACTAAACCAAGA
GAGGAACAGTATAATTCCACTTATAGAGTTGTGAGCGTCCTGACCGTGCTGCACCAGGACTGGCTGAACGGAAAAGAATAC
AAGTGTAAGGTGTCCAACAAGGCACTCCCCGCACCAATTGAGAAGACCATTTCCAAGGCCAAGGGCCAGCCAAGAGAGCCT
CAGGTGACCTATCTGCCTCCAAGCCGGGACGAACTGACAAAGAATCAGGTCAGCCTGACTTGCCTGGTGAAGGGGTTTTAC
CCTTCTGACATCGCCGTGGAATGGGAGTCTAATGGACAGCCCGAAAACAACTACAAGACCACACCACCCGTGCTGGACAGC
GATGGCTCCTTTTTCCTGTATAGTAAACTGACCGTCGACAAGTCTCGCTGGCAGCAGGGCAACGTGTTTTCTTGCAGCGTT
ATGCATGAAGCCCTCCACAACCACTATACACAGAAAAGCCTGTCTCTCAGCCCTGGGAAGTGA.

46. The composition of claim 44, wherein the mRNA that encodes the anti-IL6R antibody light chain is codon optimized and comprises a sequence at least 80% identical to one of the following sequences:
(A)
(SEQ ID NO: 6)
ATGGCCACTGGAAGCCGGACAAGCCTGCTGCTGGCCTTTGGCCTGCTG
TGTCTGCCTTGGCTGCAGGAGGGAAGCGCATTTCCAACAATTCCTCTG
AGCGACATTCAGATGACACAGAGCCCCAGCAGCGTGTCCGCATCAGTG
GGAGACAGGGTGACTATCACATGTAGAGCTTCTCAAGGAATTAGCTCT
TGGCTGGCCTGGTATCAGCAGAAGCCAGGCAAGGCCCCCAAGCTGCTG
ATCTATGGAGCTAGCTCTCTGGAGTCTGGGGTGCCATCTAGGTTCAGT
GGCTCCGGCAGCGGAACAGACTTCACACTGACTATCAGCAGCCTGCAG
CCTGAGGACTTTGCCAGCTACTACTGCCAGCAGGCAAATAGCTTTCCC
TATACTTTCGGACAGGGCACCAAGCTGGAGATTAAGCGGACCGTTGCT
GCTCCAAGCGTGTTCATCTTCCCACCTTCCGACGAGCAGCTGAAGTCT
GGCACCGCCAGCGTGGTGTGTCTGCTGAACAATTTCTATCCCCGTGAA
GCCAAAGTGCAGTGGAAGGTGGATAACGCTCTCCAGTCTGGCAATTCC
CAGGAGAGCGTGACAGAGCAGGATTCTAAGGATTCTACCTACTCCCTG
TCCAGCACACTGACCCTGAGCAAGGCCGATTACGAAAAACACAAAGTG
TACGCCTGCGAAGTCACACACCAGGGGCTGAGCTCCCCAGTGACAAAG
AGCTTTAATAGAGGGGAGTGCTGA;
(B)
(SEQ ID NO: 7)
ATGGCTACAGGGAGCCGCACTAGCCTGCTGCTGGCTTTTGGCCTGCTG
TGCCTGCCATGGCTGCAAGAGGGGTCCGCCTTTCCTACCATCCCCCTG
TCCGATATTCAGATGACCCAGTCCCCTAGCAGCGTGTCTGCCAGCGTG
GGAGACAGGGTGACTATCACCTGTAGGGCCAGCCAGGGCATTTCTAGC
TGGCTGGCTTGGTACCAGCAGAAGCCAGGAAAGGCTCCCAAACTGCTG
ATCTACGGGGCATCCTCTCTGGAGTCCGGAGTGCCAAGCAGATTCTCT
GGGAGCGGCAGCGGGACCGATTTCACACTGACCATTAGCAGCCTGCAG
CCAGAAGACTTCGCCAGCTACTATTGTCAGCAGGCAAACTCTTTTCCT
TATACCTTCGGGCAGGGGACTAAACTGGAAATCAAGCGGACAGTGGCT
GCTCCAAGCGTGTTCATCTTCCCACCTTCCGACGAGCAGCTGAAGTCT
GGCACAGCTTCCGTGGTGTGCCTGCTGAACAACTTTTATCCAAGGGAA
GCTAAAGTGCAGTGGAAGGTGGACAACGCTCTGCAGTCTGGAAATAGC
CAGGAATCCGTGACTGAGCAGGATAGCAAAGACAGCACATACAGCCTG
TCTTCCACTCTGACCCTGAGCAAGGCAGACTACGAGAAACACAAAGTG
TATGCCTGTGAGGTGACCCATCAGGGCCTGTCTAGCCCAGTGACCAAG
TCCTTTAACAGAGGCGAATGTTGA;
(C)
(SEQ ID NO: 8)
ATGGCAACTGGATCCCGGACCTCTCTGCTGCTGGCCTTCGGACTGCTG
TGCCTGCCATGGCTGCAGGAGGGGAGCGCTTTTCCTACTATCCCCCTG
TCTGACATCCAGATGACTCAGAGCCCAAGCTCTGTGTCCGCAAGCGTG
GGGGACAGGGTGACAATCACTTGCAGGGCATCCCAGGGAATCTCCTCT
TGGCTGGCATGGTACCAGCAGAAACCTGGAAAAGCCCCAAAACTGCTG
ATTTATGGCGCTTCCAGCCTCGAATCCGGAGTGCCATCCCGGTTTTCT
GGCTCCGGCAGCGGGACAGATTTTACTCTGACCATCTCTAGCCTGCAG
CCAGAGGATTTTGCCTCCTATTATTGCCAGCAGGCCAACAGCTTTCCT
TATACCTTTGGACAGGGAACTAAGCTGGAGATCAAGAGGACAGTGGCT
GCTCCTAGCGTGTTCATCTTCCCACCTTCTGACGAACAGCTGAAGTCT
GGAACAGCCTCTGTGGTGTGCCTCCTCAACAACTTCTATCCCCGGGAG
GCTAAGGTCCAGTGGAAAGTGGACAACGCCCTGCAGTCCGGAAACTCC
CAGGAGAGCGTGACCGAGCAGGACAGCAAGGATAGCACTTATTCCCTG
TCCTCCACCCTGACTCTGTCCAAGGCCGACTACGAAAAGCACAAAGTG
TACGCCTGCGAAGTCACTCATCAGGGACTGAGCTCCCCCGTGACCAAG
AGCTTCAATAGGGGAGAATGTTAG;
or
(D)
(SEQ ID NO: 9)
ATGGCAACTGGCTCCAGGACTAGCCTGCTGCTGGCATTTGGCCTCCTG
TGTCTGCCATGGCTGCAGGAGGGCTCCGCCTTCCCAACAATTCCACTG
TCCGACATCCAGATGACACAGTCCCCTAGCAGCGTGAGCGCCTCCGTG
GGAGATAGAGTGACAATTACCTGTCGCGCAAGCCAGGGGATCAGCAGC
TGGCTGGCCTGGTATCAACAGAAACCTGGAAAAGCCCCCAAGCTCCTG
ATCTATGGCGCCAGCAGCCTGGAAAGCGGGGTTCCAAGCCGGTTTTCC
GGGTCCGGCAGCGGAACTGACTTCACCCTGACAATTTCAAGCCTGCAG
CCCGAGGATTTTGCAAGCTACTACTGTCAGCAGGCTAATAGCTTTCCT
TACACATTCGGCCAGGGCACCAAGCTCGAAATTAAAAGAACTGTGGCT
GCCCCATCCGTGTTTATCTTCCCACCCTCTGACGAACAGCTGAAGTCC
GGGACAGCCTCTGTGGTGTGCCTGCTGAACAATTTTTACCCCAGGGAG
GCTAAGGTCCAATGGAAGGTCGACAATGCTCTGCAGTCTGGAAACTCC
CAGGAGTCTGTGACTGAGCAGGACAGCAAGGACAGCACCTATAGCCTG
TCTTCCACCCTGACCCTGAGCAAGGCCGATTACGAAAAGCACAAGGTG
TATGCCTGTGAGGTGACCCACCAGGGACTGTCTAGCCCAGTGACTAAA
TCCTTTAATAGAGGCGAATGCTGA.

47. The composition of claim 34, wherein the mRNA encodes an anti-IL4Rα antibody heavy chain comprising a sequence at least 80% identical to:
(SEQ ID NO: 20)
EVQLVESGGGLEQPGGSLRLSCAGSGFTFRDYAMTWVRQAPGKGLEWVS
SISGSGGNTYYADSVKGRFTISRDNSKNTLYLQMNSLRAEDTAVYYCAK
DRLSITIRPRYYGLDVWGQGTTVTVSSASTKGPSVFPLAPCSRSTSEST
AALGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKT
KPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHY
TQKSLSLSLG.

48. The composition of claim 47, wherein the mRNA encodes an anti-IL4Rα antibody heavy chain further comprising a secretion sequence at least 80% identical to:
(SEQ ID NO: 26)
MATGSRTSLLLAFGLLCLPWLQEGSAFPTIPLS.

49. The composition of claim 34, wherein the mRNA encodes an anti-IL4Rα antibody light chain comprising a sequence at least 80% identical to:
(SEQ ID NO: 21)
DIVMTQSPLSLPVTPGEPASISCRSSQSLLYSIGYNYLDWYLQKSGQSP
QLLIYLGSNRASGVPDRFSGSGSGTDFTLKISRVEAEDVGFYYCMQALQ
TPYTFGQGTKLEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

50. The composition of claim 49, wherein the mRNA encodes an anti-IL4Rα antibody light chain further comprising a secretion sequence at least 80% identical to:
(SEQ ID NO: 26)
MATGSRTSLLLAFGLLCLPWLQEGSAFPTIPLS.

51. The composition of claim 50, wherein the mRNA that encodes the anti-IL4Rα antibody heavy chain is codon optimized and comprises a sequence at least 80% identical to one of the following sequences:
(A)
(SEQ ID NO: 10)
ATGGCCACAGGCTCTAGGACATCCCTGCTGCTGGCCTTCGGACTGCTGTGTCTGCCTTGGCTGCAGGAGGGATCTGCATTCC
CAACTATCCCTCTGTCCGAGGTTCAGCTGGTGGAAAGCGGGGGAGGGCTGGAGCAGCCTGGGGGGTCCCTGAGACTGTCCTG
CGCTGGATCCGGCTTCACTTTTCGCGATTATGCCATGACATGGGTGCGGCAGGCCCCCGGCAAAGGACTGGAGTGGGTTTCC
AGCATTTCTGGCAGCGGAGGGAACACCTACTATGCCGATAGCGTGAAGGGAAGGTTTACAATCAGCCGCGATAACAGCAAGA
ATACCCTCTATCTGCAGATGAATTCTCTGAGGGCAGAGGACACTGCCGTGTATTATTGCGCAAAGGATAGGCTGAGCATCAC
TATCCGCCCACGCTACTACGGGCTGGACGTGTGGGGGCAGGGAACTACCGTTACCGTGTCTTCCGCCAGCACAAAGGGACCT
TCTGTGTTCCCCCTGGCTCCCTGTAGCAGATCCACCTCTGAGAGCACCGCTGCCCTGGGATGCCTGGTGAAGGATTATTTCC
CAGAGCCCGTGACTGTGAGCTGGAATTCAGGCGCACTCACCTCTGGGGTGCACACCTTCCCTGCCGTGCTGCAGTCCAGCGG
CCTGTATTCTCTCTCCAGCGTCGTGACCGTGCCTTCCAGTAGCCTGGGAACTAAAACATATACCTGTAACGTGGATCACAAG
CCCTCCAATACCAAGGTGGACAAGCGGGTCGAGAGCAAGTACGGACCCCCATGTCCTCCCTGTCCAGCTCCTGAGTTCCTGG
GGGGCCCTTCAGTGTTCCTGTTTCCCCCTAAGCCAAAGGACACTCTCATGATCTCCAGGACTCCAGAGGTGACATGCGTGGT
GGTGGATGTCAGCCAGGAGGATCCAGAGGTCCAGTTCAATTGGTACGTCGACGGGGTGGAGGTGCATAACGCCAAGACTAAG
CCCCGCGAGGAACAGTTTAATTCCACTTACAGGGTGGTCTCTGTGCTGACTGTCCTGCATCAGGATTGGCTGAACGGAAAGG
AGTATAAGTGCAAAGTGTCTAATAAGGGGCTGCCCAGCTCCATCGAGAAAACAATCTCTAAGGCTAAGGGGCAGCCTCGGGA
GCCTCAGGTGTACACTCTGCCCCCTTCACAGGAAGAGATGACCAAAAATCAGGTGTCCCTGACTTGCCTGGTGAAGGGGTTT
TATCCCTCTGACATCGCAGTGGAATGGGAGTCCAACGGCCAGCCTGAAAACAACTATAAGACAACCCCTCCCGTGCTGGATA
GCGACGGGAGCTTTTTCCTGTACAGCAGACTGACTGTGGATAAATCTAGGTGGCAGGAGGGAAACGTGTTTTCTTGCAGCGT
CATGCACGAAGCCCTGCACAATCACTACACACAGAAATCCCTGTCCCTGTCCCTGGGCTGA;
(B)
(SEQ ID NO: 11)
ATGGCTACCGGGTCCAGGACATCTCTGCTGCTGGCCTTCGGACTGCTGTGCCTGCCATGGCTGCAGGAAGGCTCAGCCTTTC
CAACAATCCCACTGTCCGAAGTGCAGCTGGTGGAGAGCGGCGGCGGCCTTGAACAACCTGGAGGCTCTTTGAGACTGTCATG
CGCCGGGTCCGGATTTACCTTTCGCGACTACGCAATGACTTGGGTGCGCCAGGCTCCCGGAAAGGGACTGGAATGGGTTTCC
TCTATTAGCGGGTCCGGCGGCAACACTTATTACGCAGATAGCGTGAAGGGGCGCTTCACTATTAGCAGGGACAATTCTAAAA
ACACCCTGTACCTGCAGATGAACAGCTTAAGAGCCGAAGACACAGCTGTGTACTACTGCGCTAAAGACAGACTCTCCATTAC
AATCCGCCCAAGGTATTACGGCCTGGACGTGTGGGGCCAGGGAACAACAGTGACCGTGAGCTCTGCTTCCACTAAGGGCCCT
AGCGTGTTCCCCCTGGCTCCATGCTCCCGCAGCACATCAGAGTCTACCGCCGCACTGGGATGTCTGGTGAAGGATTACTTCC
CCGAGCCTGTGACTGTGAGCTGGAATAGCGGGGCCCTGACCTCTGGAGTTCATACATTCCCAGCCGTGCTGCAGTCTTCCGG
CCTGTACTCTCTGAGCTCTGTGGTGACCGTCCCATCCTCTTCTCTGGGCACAAAGACCTACACATGTAACGTTGACCACAAG
CCATCCAATACCAAGGTGGACAAGAGAGTGGAATCCAAGTATGGCCCTCCTTGTCCCCCTTGTCCTGCTCCAGAGTTCCTGG
GAGGGCCATCCGTCTTCCTCTTCCCTCCCAAGCCTAAGGATACACTGATGATCTCCAGGACCCCTGAAGTGACATGTGTCGT
GGTGGACGTGAGCCAAGAAGACCCCGAGGTGCAGTTCAACTGGTACGTGGACGGAGTCGAGGTGCACAACGCTAAAACAAAG
CCCCGCGAGGAGCAGTTCAACTCCACATACCGGGTGGTCTCAGTGCTGACTGTGCTTCATCAGGATTGGCTGAATGGGAAGG
AGTACAAGTGCAAGGTGAGCAACAAGGGACTGCCATCTAGCATCGAGAAAACAATCAGCAAGGCTAAGGGACAGCCAAGGGA
ACCTCAGGTGTATACTCTGCCACCCTCCCAGGAAGAGATGACTAAGAATCAGGTCTCCCTGACCTGTCTGGTGAAGGGATTC
TACCCTAGCGACATTGCTGTCGAGTGGGAGTCCAACGGGCAGCCAGAAAATAATTACAAGACCACACCTCCAGTGCTGGACA
GCGATGGATCCTTCTTCCTGTACTCTCGGCTGACCGTGGATAAGAGCCGGTGGCAGGAGGGCAACGTTTTCTCTTGCAGCGT
GATGCACGAGGCTCTGCATAATCACTATACACAGAAGTCTCTAAGCCTGTCTCTGGGATGA;
(C)
(SEQ ID NO: 12)
ATGGCTACAGGATCCCGGACTAGCCTGCTGCTGGCCTTCGGCCTGTTGTGCCTGCCTTGGCTGCAGGAGGGGTCTGCCTTTC
CAACAATCCCACTGTCTGAGGTCCAGCTGGTGGAGTCCGGCGGAGGGCTAGAACAGCCTGGGGGATCTCTGAGGCTCTCTTG
CGCAGGATCCGGCTTTACATTCAGAGACTACGCAATGACTTGGGTCAGACAGGCCCCTGGAAAGGGGCTGGAGTGGGTTTCC
AGCATTTCCGGATCCGGGGGCAACACATATTACGCTGACTCTGTGAAGGGCAGGTTCACAATCAGCAGGGATAACTCCAAGA
ACACCCTCTATCTGCAGATGAACTCCCTGCGGGCCGAGGATACCGCAGTGTACTACTGTGCCAAAGATAGGCTGAGCATCAC
AATCCGCCCTAGGTATTATGGGCTCGACGTGTGGGGCCAGGGAACTACAGTGACAGTGTCCTCGGCATCCACCAAAGGCCCC
TCCGTTTTCCCCCTGGCACCCTGTAGCCGCTCTACTTCTGAGAGTACTGCTGCCCTGGGCTGCCTGGTGAAGGATTACTTTC
CAGAGCCCGTCACAGTGTCCTGGAATTCTGGGGCTCTGACTTCTGGCGTGCACACATTCCCCGCAGTGCTGCAGTCTTCTGG
CCTGTACTCTCTGTCTTCTGTGGTGACCGTGCCCAGCAGCAGCCTGGGCACTAAGACATATACCTGTAATGTTGACCACAAA
CCTTCCAACACTAAGGTGGACAAGAGGGTGGAGTCTAAGTATGGACCTCCCTGTCCACCTTGTCCTGCTCCAGAGTTCCTCG
GGGGACCAAGCGTTTTCCTGTTCCCCCCAAAGCCAAAGGACACTCTGATGATTAGCCGCACTCCCGAAGTGACTTGTGTTGT
GGTGGACGTCTCTCAGGAGGATCCTGAGGTGCAGTTTAATTGGTACGTGGATGGCGTGGAGGTGCACAACGCCAAGACAAAA
CCACGGGAGGAACAGTTCAATAGCACCTATAGGGTCGTGTCCGTCCTGACAGTGCTCCACCAGGATTGGCTCAACGGAAAAG
AATACAAATGCAAGGTGTCTAACAAGGGGCTGCCTTCCAGCATCGAGAAGACTATTAGCAAGGCAAAGGGGCAGCCAAGAGA
GCCTCAGGTGTATACCCTGCCCCCATCTCAGGAGGAGATGACAAAGAACCAGGTCTCCCTGACTTGTCTGGTCAAGGGGTTC
TACCCATCTGACATCGCTGTGGAGTGGGAGAGCAACGGCCAACCCGAGAATAACTACAAAACAACCCCACCCGTGCTGGACA
GCGATGGATCCTTCTTCCTGTATTCCAGGTTGACCGTGGACAAATCTCGCTGGCAGGAGGGAAACGTTTTCTCTTGCTCCGT
GATGCACGAGGCCCTGCACAATCACTACACACAGAAATCTCTCTCTCTGTCTCTGGGGTGA;
or
(D)
(SEQ ID NO: 13).
ATGGCTACAGGGTCTCGGACAAGTCTGCTGCTGGCATTCGGGCTGCTGTGCCTGCCATGGCTGCAAGAGGGAAGCGCATTCC
CAACCATTCCACTCAGCGAGGTGCAGCTGGTCGAAAGCGGGGGGGGACTGGAACAACCTGGAGGATCCCTGCGGCTGTCATG
CGCAGGCTCCGGCTTTACCTTCAGGGACTACGCCATGACATGGGTGAGACAGGCTCCTGGGAAGGGGCTCGAGTGGGTGAGC
AGCATTTCCGGAAGCGGGGGAAACACCTATTACGCAGATAGTGTTAAGGGCCGCTTTACTATCTCTAGGGACAATTCCAAGA
ACACCCTGTACCTGCAGATGAACAGCCTGCGAGCCGAGGACACCGCAGTGTATTACTGTGCCAAGGACCGGCTCTCTATTAC
CATTAGACCTAGGTATTACGGGCTGGACGTGTGGGGACAGGGAACAACAGTGACCGTGTCTTCTGCCTCCACAAAAGGGCCC
TCTGTGTTCCCTCTGGCACCTTGCTCCAGGTCTACCTCCGAGAGCACAGCTGCACTGGGATGTCTGGTTAAAGATTACTTTC
CAGAACCAGTTACTGTGAGCTGGAACTCTGGAGCTCTGACCTCCGGAGTTCACACATTCCCTGCAGTGCTGCAGTCTAGCGG
CCTGTATTCCCTGTCCTCCGTCGTGACCGTGCCTTCCTCCTCTCTGGGCACTAAGACCTACACTTGCAACGTGGATCACAAA
CCTAGCAATACAAAGGTCGATAAACGGGTTGAGAGCAAATACGGCCCTCCATGTCCTCCTTGTCCAGCCCCTGAATTCCTGG
GCGGACCCTCCGTTTTCCTGTTCCCACCCAAGCCCAAGGACACACTGATGATTTCTAGGACTCCTGAAGTGACATGCGTGGT
CGTGGATGTCTCCCAGGAGGATCCAGAAGTCCAGTTCAATTGGTACGTGGATGGAGTGGAGGTGCACAATGCCAAGACAAAG
CCAAGGGAGGAGCAGTTTAACTCTACTTACAGAGTGGTGAGCGTGCTCACAGTGCTGCATCAGGATTGGCTCAACGGAAAAG
AGTACAAGTGTAAGGTCAGCAATAAGGGCCTGCCATCCTCCATTGAGAAAACCATCTCCAAGGCAAAGGGGCAGCCAAGAGA
ACCTCAGGTCTACACCCTGCCACCATCTCAAGAGGAGATGACCAAGAATCAGGTGAGCCTCACTTGCCTGGTGAAGGGATTC
TACCCTAGCGACATTGCCGTGGAGTGGGAATCTAACGGGCAGCCAGAGAACAACTACAAGACAACTCCTCCCGTGCTGGATA
GCGACGGGTCTTTCTTCCTGTATAGCAGGCTGACAGTGGATAAGAGCCGCTGGCAAGAGGGCAACGTCTTTTCTTGTTCCGT
CATGCACGAGGCTCTGCATAACCACTATACCCAGAAGTCACTGTCCCTCTCCCTGGGGTGA.

52. The composition of claim 50, wherein the mRNA that encodes the anti-IL4Rα antibody light chain is codon optimized and comprises a sequence at least 80% identical to one of the following sequences:
(A)
(SEQ ID NO: 14)
ATGGCCACAGGCTCTAGGACATCCCTGCTGCTGGCCTTCGGACTGCTGT
GTCTGCCTTGGCTGCAGGAGGGATCTGCATTCCCAACTATCCCTCTGTC
CGATATTGTGATGACCCAGAGCCCCCTGAGCCTGCCAGTGACTCCTGGG
GAGCCCGCATCTATCAGCTGCCGGTCCTCTCAGTCTCTGCTGTATTCTA
TCGGGTACAACTACCTGGATTGGTACCTGCAGAAAAGTGGGCAGAGCCC
CCAGCTGCTCATCTATCTGGGGTCCAACAGGGCTAGTGGCGTGCCAGAC
CGGTTCTCCGGATCCGGCTCCGGAACAGACTTTACACTGAAAATTAGCC
GCGTGGAGGCCGAGGACGTGGGGTTTTATTATTGTATGCAGGCCCTGCA
GACCCCATACACATTTGGCCAGGGGACAAAGCTGGAAATTAAGCGCACT
GTGGCCGCTCCGTCTGTGTTCATCTTTCCTCCCAGCGATGAACAGCTGA
AGTCTGGGACCGCTAGCGTCGTGTGCCTGCTGAACAATTTTTACCCCAG
GGAGGCTAAGGTGCAGTGGAAGGTGGACAATGCCCTGCAGAGCGGGAAC
AGCCAGGAGAGTGTTACTGAGCAGGATTCTAAAGATTCCACCTATTCCC
TGTCTTCCACCCTGACTCTGTCTAAGGCCGATTACGAAAAACATAAGGT
GTACGCATGCGAGGTGACCCACCAGGGGCTGAGCTCTCCCGTGACTAAG
AGCTTCAATCGCGGAGAGTGCTGA;
(B)
(SEQ ID NO: 15)
ATGGCTACAGGCAGCAGAACCAGCCTGCTGCTGGCATTTGGCCTGCTGT
GCCTGCCTTGGCTGCAGGAGGGGAGCGCTTTTCCCACAATTCCTCTGTC
TGATATCGTCATGACCCAATCTCCCCTGTCCCTGCCTGTGACTCCAGGA
GAGCCCGCTAGCATTTCTTGCAGGTCTTCCCAGAGCCTGCTGTACAGCA
TCGGCTATAACTACCTGGATTGGTATCTGCAGAAAAGCGGGCAGTCTCC
TCAGCTGCTGATCTACCTGGGCTCTAACAGAGCCTCTGGGGTCCCCGAC
AGGTTTTCCGGAAGCGGCTCTGGCACCGACTTTACTCTCAAAATCAGCC
GCGTGGAGGCAGAGGACGTGGGCTTCTATTACTGCATGCAGGCCCTGCA
GACACCATATACATTCGGACAGGGGACCAAGCTGGAGATTAAGAGAACA
GTGGCTGCCCCAAGCGTGTTTATCTTTCCTCCCTCCGATGAACAGCTGA
AAAGCGGCACTGCTTCCGTGGTGTGCCTGCTGAATAATTTCTACCCTAG
AGAGGCCAAAGTCCAGTGGAAGGTGGACAACGCCCTGCAGAGCGGGAAC
AGCCAGGAAAGTGTCACCGAGCAGGATTCCAAGGATTCCACATATTCTC
TGTCCAGCACTCTGACACTGTCCAAGGCAGACTACGAAAAACACAAGGT
CTACGCCTGCGAAGTGACCCACCAGGGACTGTCTAGCCCTGTGACTAAG
TCTTTTAATAGGGGGGAGTGTTAG;
(C)
(SEQ ID NO: 16)
ATGGCTACTGGCAGCAGAACCAGCCTGCTGCTGGCATTCGGGCTGCTCT
GCCTGCCATGGCTGCAGGAGGGATCCGCCTTCCCAACTATCCCCCTGAG
CGATATCGTGATGACCCAGTCTCCCCTGAGCCTGCCAGTTACACCCGGC
GAACCTGCTAGCATCAGCTGCAGATCCTCCCAGTCTCTCCTGTACTCCA
TCGGGTACAATTATCTGGATTGGTATCTGCAGAAGTCTGGCCAATCCCC
CCAGCTGCTGATCTACCTGGGCTCCAACAGAGCAAGCGGCGTGCCCGAT
AGATTCAGCGGCAGCGGGAGCGGCACTGATTTTACTCTGAAGATCAGCA
GGGTGGAGGCCGAAGATGTGGGATTTTACTACTGCATGCAAGCACTGCA
GACTCCTTACACATTCGGCCAGGGAACTAAGCTGGAGATCAAAAGAACC
GTGGCAGCTCCAAGCGTCTTCATTTTCCCACCTTCTGACGAGCAGCTGA
AGTCCGGCACAGCTTCCGTCGTGTGCCTCCTGAACAACTTCTACCCCAG
GGAGGCAAAGGTGCAATGGAAAGTGGACAACGCTCTGCAGAGCGGAAAC
AGTCAGGAGTCCGTGACCGAGCAGGACAGCAAAGACTCCACTTACAGCC
TGAGCTCTACTCTGACCCTGAGCAAAGCTGACTACGAGAAGCATAAGGT
GTATGCTTGCGAGGTCACCCACCAGGGCCTCTCTTCTCCCGTGACCAAG
AGCTTCAACAGAGGCGAGTGCTGA;
or
(D)
(SEQ ID NO: 17)
ATGGCAACTGGAAGCAGGACCTCCCTGCTCCTGGCTTTCGGCCTGCTCT
GTCTGCCATGGCTGCAAGAAGGATCTGCCTTTCCTACAATTCCACTGTC
CGACATCGTGATGACACAGTCCCCCCTGTCTCTGCCTGTCACCCCAGGC
GAACCAGCCTCTATTTCTTGTCGGTCCTCTCAGTCCCTGCTGTATAGCA
TCGGATATAATTATCTGGACTGGTACCTGCAAAAATCCGGCCAGTCTCC
TCAGCTGCTGATCTATCTGGGCTCCAACCGGGCTAGCGGAGTCCCAGAC
CGGTTTTCCGGGTCTGGCAGTGGGACAGATTTTACACTGAAAATTTCCC
GGGTGGAGGCTGAGGACGTGGGATTTTACTACTGTATGCAGGCCCTGCA
AACCCCATATACTTTCGGACAGGGAACAAAGCTGGAGATCAAAAGAACC
GTGGCCGCCCCCAGCGTTTTCATCTTCCCACCAAGCGACGAGCAGCTCA
AATCTGGGACCGCTAGCGTGGTCTGTCTGCTGAATAACTTCTACCCAAG
GGAAGCAAAGGTGCAGTGGAAGGTCGACAACGCACTGCAGAGCGGGAAC
TCCCAGGAGAGCGTGACTGAACAGGACAGCAAGGACAGCACCTATAGCC
TCAGCAGCACTCTGACCCTGTCTAAAGCTGATTACGAAAAACACAAGGT
GTATGCTTGTGAAGTGACTCACCAGGGCCTGTCTTCCCCTGTTACAAAG
TCCTTCAATAGAGGAGAATGTTAA.

53. The composition of any one of claims 31-52, wherein the mRNA comprises a 5′ UTR and a 3′UTR sequence.

54. The composition of claim 53, wherein the 5′ UTR sequence comprises a sequence with at least 80% identity to
(SEQ ID NO: 27)
GGACAGATCGCCTGGAGACGCCATCCACGCTGTTTTGACCTCCATAGAA
GACACCGGGACCGATCCAGCCTCCGCGGCCGGGAACGGTGCATTGGAAC
GCGGATTCCCCGTGCCAAGAGTGACTCACCGTCCTTGACACG.

55. The composition of any one of claim 53 or 54, wherein the 3′ UTR sequence comprises a sequence with at least 80% identity to:
(SEQ ID NO: 28)
CGGGTGGCATCCCTGTGACCCCTCCCCAGTGCCTCTCCTGGCCCTGGAA
GTTGCCACTCCAGTGCCCACCAGCCTTGTCCTAATAAAATTAAGTTGCA
TC.


SEQUENCE LISTING (29 sequences)

SEQ ID NO: 1 - protein, 874 aa, Shigella virus Sf6
MQDLHAIQLQLEEEMFNGGIRRFEADQQRQIAAGSESDTAWNRRLLSELIAPMAEGIQAYKEEYEGKKGRAPRALAFLQCVENEVAAYITMKVVMDMLNTDATLQAIAMSVAERIEDQVRFSKLEGHAAKYFEKVKKSLKASRTKSYRHAHNVAVVAEKSVAEKDADFDRWEAWPKETQLQIGTTLLEILEGSVFYNGEPVFMRAMRTYGGKTIYYLQTSESVGQWISAFKEHVAQLSPAYAPCVIPPRPWRTPFNGGFHTEKVASRIRLVKGNREHVRKLTQKQMPKVYKAINALQNTQWQINKDVLAVIEEVIRLDLGYGVPSFKPLIDKENKPANPVPVEFQHLRGRELKEMLSPEQWQQFINWKGECARLYTAETKRGSKSAAVVRMVGQARKYSAFESIYFVYAMDSRSRVYVQSSTLSPQSNDLGKALLRFTEGRPVNGVEALKWFCINGANLWGWDKKTFDVRVSNVLDEEFQDMCRDIAADPLTFTQWAKADAPYEFLAWCFEYAQYLDLVDEGRADEFRTHLPVHQDGSCSGIQHYSAMLRDEVGAKAVNLKPSDAPQDIYGAVAQVVIKKNALYMDADDATTFTSGSVTLSGTELRAMASAWDSIGITRSLTKKPVMTLPYGSTRLTCRESVIDYIVDLEEKEAQKAVAEGRTANKVHPFEDDRQDYLTPGAAYNYMTALIWPSISEVVKAPIVAMKMIRQLARFAAKRNEGLMYTLPTGFILEQKIMATEMLRVRTCLMGDIKMSLQVETDIVDEAAMMGAAAPNFVHGHDASHLILTVCELVDKGVTSIAVIHDSFGTHADNTLTLRVALKGQMVAMYIDGNALQKLLEEHEVRWMVDTGIEVPEQGEFDLNEIMDSEYVFA

SEQ ID NO: 2 - DNA, 1440 bp, synthetic construct
  misc_feature 1..1440: note = source = /note="Description of Artificial Sequence: Syntheticpolynucleotide"
  source 1..1440: mol_type = other DNA
atggccactggaagccggacaagcctgctgctggcctttggcctgctgtgtctgccttggctgcaggagggaagcgcatttccaacaattcctctgagcgaagtgcagctggtggagtctggaggaggcctcgtgcagccaggcagatccctgaggctctcctgcgccgctagcagattcactttcgacgactacgccatgcactgggtgcggcaggcacctggcaagggactggaatgggtgtccggcatttcttggaacagcgggcggatcgggtacgcggacagcgtgaaaggaaggtttacaatctcccgggacaatgctgagaactccctgttcctgcagatgaacggcctgagagctgaagacacagcactgtactattgcgcaaaaggccgcgactcctttgacatctgggggcagggcacaatggtgaccgtgtctagcgcctccacaaaaggacctagcgttttcccactggctccatctagcaagtctacatccgggggcaccgccgctctgggctgtctggtgaaggattacttccctgagcccgtcactgtcagctggaactccggagctctgacctcaggcgtgcacacttttcccgctgtgctgcagagctctggcctgtacagcctgagcagcgttgtgaccgtgcctagctcatccctcggcacccagacctatatctgcaacgtcaaccacaaaccttccaacaccaaagtggacaagaaagtggaacctaagtcctgcgataagactcatacttgccctccttgtccagcaccagagctgctgggggggccaagcgtgtttctctttccacctaagcctaaagacaccctgatgatctccaggaccccagaggtgacatgtgtggtggtggacgtgtctcatgaggaccctgaggtgaaattcaattggtatgtggacggcgttgaggttcacaacgcaaagaccaagccaagggaggagcagtataatagcacctatcgcgtggtgtccgtcctgacagtgctgcaccaggactggctgaacgggaaggagtataagtgtaaagtgagcaacaaggcactgcctgctcctatcgagaagactatcagcaaagctaaaggacagccaagagagccccaggtgacctacctgccaccttctcgggacgaactgaccaaaaaccaggtgagcctgacttgcctggtgaagggcttttatccctctgatattgcagtggagtgggagagtaacgggcagcccgagaacaactacaagactactccaccagttctggattccgacggcagcttcttcctgtatagcaaactgacagtggacaagtcccggtggcagcagggcaacgtttttagctgcagcgtgatgcatgaggctctgcacaaccattacacacagaagtctctgtctctgtcccccggaaagtga

SEQ ID NO: 3 - DNA, 1440 bp, synthetic construct
  misc_feature 1..1440: note = source = /note="Description of Artificial Sequence: Syntheticpolynucleotide"
  source 1..1440: mol_type = other DNA
atggccaccgggtctcggacaagcctcctgctcgcattcgggctcctgtgtctgccttggctgcaagaaggatccgcatttcccaccattccactgtctgaggtgcagctggtcgagtctggaggaggactggtgcagcctggcaggtctctgaggctgtcttgcgctgccagccggtttacctttgatgattacgccatgcactgggtgaggcaggctcccggcaaaggactggaatgggtgtccggaatttcctggaatagtggcaggatcggctatgccgactctgtcaaaggccggtttacaatctcccgcgacaacgctgagaactccctgttcctgcagatgaacggcctgagagctgaagataccgccctgtactattgcgccaaggggcgcgacagcttcgacatttggggccagggaaccatggtgactgtgagcagcgcatccacaaaagggccctccgtgttccccctggcaccttccagtaaatccacttctggcggaacagcagctctcggctgtctggtgaaggattatttccccgagccagtgacagtgtcttggaattctggcgcactcaccagtggagtccacacttttccagccgtgctgcagagctccggactgtattccctgagctccgtcgtgacagtgccatcctcttctctgggaactcagacatatatttgcaacgttaatcataagccttctaacaccaaggtggataagaaagtggagcccaaatcctgcgacaagacacacacttgcccaccatgccctgcccctgaactgctgggaggaccaagcgtgtttctcttccctcctaagcctaaggataccctgatgatctctaggaccccagaggtgacatgcgtggtggttgacgtctcccatgaagatcctgaagtgaaatttaactggtacgtggacggagtggaagtgcacaatgcaaagaccaaaccccgcgaggaacagtacaactccacttaccgggtggtttccgtgctgacagtgctgcaccaggattggctgaacggaaaagagtataagtgcaaagtgagcaataaggccctgcccgcccctattgagaagaccatctctaaggctaaaggccagcctcgcgaaccccaggttacctatctgcctccaagcagagatgagctcaccaaaaaccaggtgtctctgacctgtctggtgaaaggcttctatccaagcgatatcgccgtggagtgggagtccaacggacagccagaaaacaattacaagactaccccacctgtcctggacagcgacgggagcttctttctgtactctaagctgacagtcgacaaaagccggtggcagcaaggcaacgtcttcagctgcagcgtcatgcacgaggccctgcataatcattatactcagaagtctctgagcctgagccctggcaagtag

SEQ ID NO: 4 - DNA, 1440 bp, synthetic construct
  misc_feature 1..1440: note = source = /note="Description of Artificial Sequence: Syntheticpolynucleotide"
  source 1..1440: mol_type = other DNA
atggccactggaagcagaacctccctgctgctggcattcggactgctgtgcctgccatggctgcaggagggatccgctttcccaaccatccccctcagcgaggtgcagctcgttgaatctggaggaggactggtgcaaccaggacgctccctgagactgtcttgtgctgcttccaggtttacttttgacgattatgctatgcactgggtgagacaggccccaggaaaaggactggaatgggtgtctggaatttcttggaacagcggacgcattggctacgccgactctgtgaagggaaggtttactatctccagggataacgcggaaaactccctcttcctccagatgaacggcctgagggcagaggacaccgctctgtactactgcgccaaaggaagagatagcttcgatatctggggacaggggaccatggtgacagtttccagcgctagcaccaagggccccagcgtgttcccactggccccatcctccaagagcacttctggcgggactgctgcactgggctgcctggtgaaggattatttccctgagcctgtgacagtgagctggaactcaggagcactgacttccggggtgcatacattccccgctgtgctgcagtcttctgggctgtattccctcagcagcgtggtgaccgtcccttcctcaagcctgggaacccagacatatatttgtaacgtgaaccacaagccaagcaatacaaaggtggataagaaggtggagcctaagtcctgtgacaaaacacacacatgtcccccatgtccagctcctgaactgcttggcggaccatccgtcttcctgtttccacccaaaccaaaggatacactgatgatcagccggacaccagaggtgacttgcgtcgtcgtggacgtcagccatgaagaccccgaggtgaagtttaattggtatgtggacggggtggaggtccacaacgccaagaccaaacccagggaggagcagtacaactccacttatcgcgtggtttctgtgctgacagtcctgcaccaggattggctgaacgggaaggagtacaaatgcaaagtgtccaataaggccctgcccgccccaatcgagaaaactatttcaaaggccaaaggacagcccagagagccacaggtgacctacctccctccttccagggacgagctcactaagaatcaggtgtctctgacttgcctggtgaaaggcttttatccttctgacatcgcagtggagtgggagagcaatggccagcccgagaacaattataaaacaacaccacccgtcctggactctgatggcagctttttcctgtatagcaagctgacagtggacaaatcacgctggcagcaggggaatgtcttcagctgtagcgtgatgcacgaagctctgcacaatcactatacacagaagtccctgtccctgagcccaggaaaataa

SEQ ID NO: 5 - DNA, 1440 bp, synthetic construct
  misc_feature 1..1440: note = source = /note="Description of Artificial Sequence: Syntheticpolynucleotide"
  source 1..1440: mol_type = other DNA
atggctaccggcagcaggactagcctgctgctggctttcggcctgctgtgtctgccttggctgcaagaggggtccgctttccctactatccctctgtccgaagtgcagctggtcgagagcggagggggcctggtgcagcctggaagaagtctgcgcctgtcctgcgcagcaagcaggtttacatttgacgactacgcaatgcactgggtgcgccaagctccaggcaaaggcttagaatgggtgtctggcatcagctggaactcagggcggatcggctacgcagacagcgtgaagggcaggttcactatctctagggacaacgccgagaactccctgttcctgcagatgaatgggctgcgggcagaagacactgcactgtattattgtgctaaggggagagactctttcgacatctggggccagggcacaatggtgactgtgtcctctgcctctaccaagggcccttccgtgttcccactggcaccaagcagcaaatccacatccggggggaccgcagctctcggatgtctggtgaaagactatttccctgagcccgtcacagtgtcttggaattccggcgccctgacaagcggcgtgcacacttttcctgccgttctgcagagctccggcctatactccctgtccagcgtggtgacagtcccttctagcagtctgggcacacagacttatatttgcaacgtgaatcacaagccatctaacaccaaggtggataagaaggtggaaccaaagtcctgtgataaaacccatacctgtcctccatgtccagctcctgaactcctggggggaccctctgtgttcctgttcccacctaagcctaaagacactctgatgatttccagaactcctgaggtgacttgcgtggtggtggatgtgtcccatgaggatcctgaggtcaagttcaactggtacgtggacggagtcgaggtgcataacgctaaaactaaaccaagagaggaacagtataattccacttatagagttgtgagcgtcctgaccgtgctgcaccaggactggctgaacggaaaagaatacaagtgtaaggtgtccaacaaggcactccccgcaccaattgagaagaccatttccaaggccaagggccagccaagagagcctcaggtgacctatctgcctccaagccgggacgaactgacaaagaatcaggtcagcctgacttgcctggtgaaggggttttacccttctgacatcgccgtggaatgggagtctaatggacagcccgaaaacaactacaagaccacaccacccgtgctggacagcgatggctcctttttcctgtatagtaaactgaccgtcgacaagtctcgctggcagcagggcaacgtgttttcttgcagcgttatgcatgaagccctccacaaccactatacacagaaaagcctgtctctcagccctgggaagtga

SEQ ID NO: 6 - DNA, 744 bp, synthetic construct
  misc_feature 1..744: note = source = /note="Description of Artificial Sequence: Syntheticpolynucleotide"
  source 1..744: mol_type = other DNA
atggccactggaagccggacaagcctgctgctggcctttggcctgctgtgtctgccttggctgcaggagggaagcgcatttccaacaattcctctgagcgacattcagatgacacagagccccagcagcgtgtccgcatcagtgggagacagggtgactatcacatgtagagcttctcaaggaattagctcttggctggcctggtatcagcagaagccaggcaaggcccccaagctgctgatctatggagctagctctctggagtctggggtgccatctaggttcagtggctccggcagcggaacagacttcacactgactatcagcagcctgcagcctgaggactttgccagctactactgccagcaggcaaatagctttccctatactttcggacagggcaccaagctggagattaagcggaccgttgctgctccaagcgtgttcatcttcccaccttccgacgagcagctgaagtctggcaccgccagcgtggtgtgtctgctgaacaatttctatccccgtgaagccaaagtgcagtggaaggtggataacgctctccagtctggcaattcccaggagagcgtgacagagcaggattctaaggattctacctactccctgtccagcacactgaccctgagcaaggccgattacgaaaaacacaaagtgtacgcctgcgaagtcacacaccaggggctgagctccccagtgacaaagagctttaatagaggggagtgctga

SEQ ID NO: 7 - DNA, 744 bp, synthetic construct
  misc_feature 1..744: note = source = /note="Description of Artificial Sequence: Syntheticpolynucleotide"
  source 1..744: mol_type = other DNA
atggctacagggagccgcactagcctgctgctggcttttggcctgctgtgcctgccatggctgcaagaggggtccgcctttcctaccatccccctgtccgatattcagatgacccagtcccctagcagcgtgtctgccagcgtgggagacagggtgactatcacctgtagggccagccagggcatttctagctggctggcttggtaccagcagaagccaggaaaggctcccaaactgctgatctacggggcatcctctctggagtccggagtgccaagcagattctctgggagcggcagcgggaccgatttcacactgaccattagcagcctgcagccagaagacttcgccagctactattgtcagcaggcaaactcttttccttataccttcgggcaggggactaaactggaaatcaagcggacagtggctgctccaagcgtgttcatcttcccaccttccgacgagcagctgaagtctggcacagcttccgtggtgtgcctgctgaacaacttttatccaagggaagctaaagtgcagtggaaggtggacaacgctctgcagtctggaaatagccaggaatccgtgactgagcaggatagcaaagacagcacatacagcctgtcttccactctgaccctgagcaaggcagactacgagaaacacaaagtgtatgcctgtgaggtgacccatcagggcctgtctagcccagtgaccaagtcctttaacagaggcgaatgttga

SEQ ID NO: 8 - DNA, 744 bp, synthetic construct
  misc_feature 1..744: note = source = /note="Description of Artificial Sequence: Syntheticpolynucleotide"
  source 1..744: mol_type = other DNA
atggcaactggatcccggacctctctgctgctggccttcggactgctgtgcctgccatggctgcaggaggggagcgcttttcctactatccccctgtctgacatccagatgactcagagcccaagctctgtgtccgcaagcgtgggggacagggtgacaatcacttgcagggcatcccagggaatctcctcttggctggcatggtaccagcagaaacctggaaaagccccaaaactgctgatttatggcgcttccagcctcgaatccggagtgccatcccggttttctggctccggcagcgggacagattttactctgaccatctctagcctgcagccagaggattttgcctcctattattgccagcaggccaacagctttccttatacctttggacagggaactaagctggagatcaagaggacagtggctgctcctagcgtgttcatcttcccaccttctgacgaacagctgaagtctggaacagcctctgtggtgtgcctcctcaacaacttctatccccgggaggctaaggtccagtggaaagtggacaacgccctgcagtccggaaactcccaggagagcgtgaccgagcaggacagcaaggatagcacttattccctgtcctccaccctgactctgtccaaggccgactacgaaaagcacaaagtgtacgcctgcgaagtcactcatcagggactgagctcccccgtgaccaagagcttcaataggggagaatgttag

SEQ ID NO: 9 - DNA, 744 bp, synthetic construct
  misc_feature 1..744: note = source = /note="Description of Artificial Sequence: Syntheticpolynucleotide"
  source 1..744: mol_type = other DNA
atggcaactggctccaggactagcctgctgctggcatttggcctcctgtgtctgccatggctgcaggagggctccgccttcccaacaattccactgtccgacatccagatgacacagtcccctagcagcgtgagcgcctccgtgggagatagagtgacaattacctgtcgcgcaagccaggggatcagcagctggctggcctggtatcaacagaaacctggaaaagcccccaagctcctgatctatggcgccagcagcctggaaagcggggttccaagccggttttccgggtccggcagcggaactgacttcaccctgacaatttcaagcctgcagcccgaggattttgcaagctactactgtcagcaggctaatagctttccttacacattcggccagggcaccaagctcgaaattaaaagaactgtggctgccccatccgtgtttatcttcccaccctctgacgaacagctgaagtccgggacagcctctgtggtgtgcctgctgaacaatttttaccccagggaggctaaggtccaatggaaggtcgacaatgctctgcagtctggaaactcccaggagtctgtgactgagcaggacagcaaggacagcacctatagcctgtcttccaccctgaccctgagcaaggccgattacgaaaagcacaaggtgtatgcctgtgaggtgacccaccagggactgtctagcccagtgactaaatcctttaatagaggcgaatgctga

SEQ ID NO: 10 - DNA, 1455 bp, synthetic construct
  misc_feature 1..1455: note = source = /note="Description of Artificial Sequence: Syntheticpolynucleotide"
  source 1..1455: mol_type = other DNA
atggccacaggctctaggacatccctgctgctggccttcggactgctgtgtctgccttggctgcaggagggatctgcattcccaactatccctctgtccgaggttcagctggtggaaagcgggggagggctggagcagcctggggggtccctgagactgtcctgcgctggatccggcttcacttttcgcgattatgccatgacatgggtgcggcaggcccccggcaaaggactggagtgggtttccagcatttctggcagcggagggaacacctactatgccgatagcgtgaagggaaggtttacaatcagccgcgataacagcaagaataccctctatctgcagatgaattctctgagggcagaggacactgccgtgtattattgcgcaaaggataggctgagcatcactatccgcccacgctactacgggctggacgtgtgggggcagggaactaccgttaccgtgtcttccgccagcacaaagggaccttctgtgttccccctggctccctgtagcagatccacctctgagagcaccgctgccctgggatgcctggtgaaggattatttcccagagcccgtgactgtgagctggaattcaggcgcactcacctctggggtgcacaccttccctgccgtgctgcagtccagcggcctgtattctctctccagcgtcgtgaccgtgccttccagtagcctgggaactaaaacatatacctgtaacgtggatcacaagccctccaataccaaggtggacaagcgggtcgagagcaagtacggacccccatgtcctccctgtccagctcctgagttcctggggggcccttcagtgttcctgtttccccctaagccaaaggacactctcatgatctccaggactccagaggtgacatgcgtggtggtggatgtcagccaggaggatccagaggtccagttcaattggtacgtcgacggggtggaggtgcataacgccaagactaagccccgcgaggaacagtttaattccacttacagggtggtctctgtgctgactgtcctgcatcaggattggctgaacggaaaggagtataagtgcaaagtgtctaataaggggctgcccagctccatcgagaaaacaatctctaaggctaaggggcagcctcgggagcctcaggtgtacactctgcccccttcacaggaagagatgaccaaaaatcaggtgtccctgacttgcctggtgaaggggttttatccctctgacatcgcagtggaatgggagtccaacggccagcctgaaaacaactataagacaacccctcccgtgctggatagcgacgggagctttttcctgtacagcagactgactgtggataaatctaggtggcaggagggaaacgtgttttcttgcagcgtcatgcacgaagccctgcacaatcactacacacagaaatccctgtccctgtccctgggctga

SEQ ID NO: 11 - DNA, 1455 bp, synthetic construct
  misc_feature 1..1455: note = source = /note="Description of Artificial Sequence: Syntheticpolynucleotide"
  source 1..1455: mol_type = other DNA
atggctaccgggtccaggacatctctgctgctggccttcggactgctgtgcctgccatggctgcaggaaggctcagcctttccaacaatcccactgtccgaagtgcagctggtggagagcggcggcggccttgaacaacctggaggctctttgagactgtcatgcgccgggtccggatttacctttcgcgactacgcaatgacttgggtgcgccaggctcccggaaagggactggaatgggtttcctctattagcgggtccggcggcaacacttattacgcagatagcgtgaaggggcgcttcactattagcagggacaattctaaaaacaccctgtacctgcagatgaacagcttaagagccgaagacacagctgtgtactactgcgctaaagacagactctccattacaatccgcccaaggtattacggcctggacgtgtggggccagggaacaacagtgaccgtgagctctgcttccactaagggccctagcgtgttccccctggctccatgctcccgcagcacatcagagtctaccgccgcactgggatgtctggtgaaggattacttccccgagcctgtgactgtgagctggaatagcggggccctgacctctggagttcatacattcccagccgtgctgcagtcttccggcctgtactctctgagctctgtggtgaccgtcccatcctcttctctgggcacaaagacctacacatgtaacgttgaccacaagccatccaataccaaggtggacaagagagtggaatccaagtatggccctccttgtcccccttgtcctgctccagagttcctgggagggccatccgtcttcctcttccctcccaagcctaaggatacactgatgatctccaggacccctgaagtgacatgtgtcgtggtggacgtgagccaagaagaccccgaggtgcagttcaactggtacgtggacggagtcgaggtgcacaacgctaaaacaaagccccgcgaggagcagttcaactccacataccgggtggtctcagtgctgactgtgcttcatcaggattggctgaatgggaaggagtacaagtgcaaggtgagcaacaagggactgccatctagcatcgagaaaacaatcagcaaggctaagggacagccaagggaacctcaggtgtatactctgccaccctcccaggaagagatgactaagaatcaggtctccctgacctgtctggtgaagggattctaccctagcgacattgctgtcgagtgggagtccaacgggcagccagaaaataattacaagaccacacctccagtgctggacagcgatggatccttcttcctgtactctcggctgaccgtggataagagccggtggcaggagggcaacgttttctcttgcagcgtgatgcacgaggctctgcataatcactatacacagaagtctctaagcctgtctctgggatga

SEQ ID NO: 12 - DNA, 1455 bp, synthetic construct
  misc_feature 1..1455: note = source = /note="Description of Artificial Sequence: Syntheticpolynucleotide"
  source 1..1455: mol_type = other DNA
atggctacaggatcccggactagcctgctgctggccttcggcctgttgtgcctgccttggctgcaggaggggtctgcctttccaacaatcccactgtctgaggtccagctggtggagtccggcggagggctagaacagcctgggggatctctgaggctctcttgcgcaggatccggctttacattcagagactacgcaatgacttgggtcagacaggcccctggaaaggggctggagtgggtttccagcatttccggatccgggggcaacacatattacgctgactctgtgaagggcaggttcacaatcagcagggataactccaagaacaccctctatctgcagatgaactccctgcgggccgaggataccgcagtgtactactgtgccaaagataggctgagcatcacaatccgccctaggtattatgggctcgacgtgtggggccagggaactacagtgacagtgtcctcggcatccaccaaaggcccctccgttttccccctggcaccctgtagccgctctacttctgagagtactgctgccctgggctgcctggtgaaggattactttccagagcccgtcacagtgtcctggaattctggggctctgacttctggcgtgcacacattccccgcagtgctgcagtcttctggcctgtactctctgtcttctgtggtgaccgtgcccagcagcagcctgggcactaagacatatacctgtaatgttgaccacaaaccttccaacactaaggtggacaagagggtggagtctaagtatggacctccctgtccaccttgtcctgctccagagttcctcgggggaccaagcgttttcctgttccccccaaagccaaaggacactctgatgattagccgcactcccgaagtgacttgtgttgtggtggacgtctctcaggaggatcctgaggtgcagtttaattggtacgtggatggcgtggaggtgcacaacgccaagacaaaaccacgggaggaacagttcaatagcacctatagggtcgtgtccgtcctgacagtgctccaccaggattggctcaacggaaaagaatacaaatgcaaggtgtctaacaaggggctgccttccagcatcgagaagactattagcaaggcaaaggggcagccaagagagcctcaggtgtataccctgcccccatctcaggaggagatgacaaagaaccaggtctccctgacttgtctggtcaaggggttctacccatctgacatcgctgtggagtgggagagcaacggccaacccgagaataactacaaaacaaccccacccgtgctggacagcgatggatccttcttcctgtattccaggttgaccgtggacaaatctcgctggcaggagggaaacgttttctcttgctccgtgatgcacgaggccctgcacaatcactacacacagaaatctctctctctgtctctggggtga

SEQ ID NO: 13 - DNA, 1455 bp, synthetic construct
  misc_feature 1..1455: note = source = /note="Description of Artificial Sequence: Syntheticpolynucleotide"
  source 1..1455: mol_type = other DNA
atggctacagggtctcggacaagtctgctgctggcattcgggctgctgtgcctgccatggctgcaagagggaagcgcattcccaaccattccactcagcgaggtgcagctggtcgaaagcggggggggactggaacaacctggaggatccctgcggctgtcatgcgcaggctccggctttaccttcagggactacgccatgacatgggtgagacaggctcctgggaaggggctcgagtgggtgagcagcatttccggaagcgggggaaacacctattacgcagatagtgttaagggccgctttactatctctagggacaattccaagaacaccctgtacctgcagatgaacagcctgcgagccgaggacaccgcagtgtattactgtgccaaggaccggctctctattaccattagacctaggtattacgggctggacgtgtggggacagggaacaacagtgaccgtgtcttctgcctccacaaaagggccctctgtgttccctctggcaccttgctccaggtctacctccgagagcacagctgcactgggatgtctggttaaagattactttccagaaccagttactgtgagctggaactctggagctctgacctccggagttcacacattccctgcagtgctgcagtctagcggcctgtattccctgtcctccgtcgtgaccgtgccttcctcctctctgggcactaagacctacacttgcaacgtggatcacaaacctagcaatacaaaggtcgataaacgggttgagagcaaatacggccctccatgtcctccttgtccagcccctgaattcctgggcggaccctccgttttcctgttcccacccaagcccaaggacacactgatgatttctaggactcctgaagtgacatgcgtggtcgtggatgtctcccaggaggatccagaagtccagttcaattggtacgtggatggagtggaggtgcacaatgccaagacaaagccaagggaggagcagtttaactctacttacagagtggtgagcgtgctcacagtgctgcatcaggattggctcaacggaaaagagtacaagtgtaaggtcagcaataagggcctgccatcctccattgagaaaaccatctccaaggcaaaggggcagccaagagaacctcaggtctacaccctgccaccatctcaagaggagatgaccaagaatcaggtgagcctcacttgcctggtgaagggattctaccctagcgacattgccgtggagtgggaatctaacgggcagccagagaacaactacaagacaactcctcccgtgctggatagcgacgggtctttcttcctgtatagcaggctgacagtggataagagccgctggcaagagggcaacgtcttttcttgttccgtcatgcacgaggctctgcataaccactatacccagaagtcactgtccctctccctggggtga

SEQ ID NO: 14 - DNA, 759 bp, synthetic construct
  misc_feature 1..759: note = source = /note="Description of Artificial Sequence: Syntheticpolynucleotide"
  source 1..759: mol_type = other DNA
atggccacaggctctaggacatccctgctgctggccttcggactgctgtgtctgccttggctgcaggagggatctgcattcccaactatccctctgtccgatattgtgatgacccagagccccctgagcctgccagtgactcctggggagcccgcatctatcagctgccggtcctctcagtctctgctgtattctatcgggtacaactacctggattggtacctgcagaaaagtgggcagagcccccagctgctcatctatctggggtccaacagggctagtggcgtgccagaccggttctccggatccggctccggaacagactttacactgaaaattagccgcgtggaggccgaggacgtggggttttattattgtatgcaggccctgcagaccccatacacatttggccaggggacaaagctggaaattaagcgcactgtggccgctccgtctgtgttcatctttcctcccagcgatgaacagctgaagtctgggaccgctagcgtcgtgtgcctgctgaacaatttttaccccagggaggctaaggtgcagtggaaggtggacaatgccctgcagagcgggaacagccaggagagtgttactgagcaggattctaaagattccacctattccctgtcttccaccctgactctgtctaaggccgattacgaaaaacataaggtgtacgcatgcgaggtgacccaccaggggctgagctctcccgtgactaagagcttcaatcgcggagagtgctga

SEQ ID NO: 15 - DNA, 759 bp, synthetic construct
  misc_feature 1..759: note = source = /note="Description of Artificial Sequence: Syntheticpolynucleotide"
  source 1..759: mol_type = other DNA
atggctacaggcagcagaaccagcctgctgctggcatttggcctgctgtgcctgccttggctgcaggaggggagcgcttttcccacaattcctctgtctgatatcgtcatgacccaatctcccctgtccctgcctgtgactccaggagagcccgctagcatttcttgcaggtcttcccagagcctgctgtacagcatcggctataactacctggattggtatctgcagaaaagcgggcagtctcctcagctgctgatctacctgggctctaacagagcctctggggtccccgacaggttttccggaagcggctctggcaccgactttactctcaaaatcagccgcgtggaggcagaggacgtgggcttctattactgcatgcaggccctgcagacaccatatacattcggacaggggaccaagctggagattaagagaacagtggctgccccaagcgtgtttatctttcctccctccgatgaacagctgaaaagcggcactgcttccgtggtgtgcctgctgaataatttctaccctagagaggccaaagtccagtggaaggtggacaacgccctgcagagcgggaacagccaggaaagtgtcaccgagcaggattccaaggattccacatattctctgtccagcactctgacactgtccaaggcagactacgaaaaacacaaggtctacgcctgcgaagtgacccaccagggactgtctagccctgtgactaagtcttttaataggggggagtgttag

SEQ ID NO: 16 - DNA, 759 bp, synthetic construct
  misc_feature 1..759: note = source = /note="Description of Artificial Sequence: Syntheticpolynucleotide"
  source 1..759: mol_type = other DNA
atggctactggcagcagaaccagcctgctgctggcattcgggctgctctgcctgccatggctgcaggagggatccgccttcccaactatccccctgagcgatatcgtgatgacccagtctcccctgagcctgccagttacacccggcgaacctgctagcatcagctgcagatcctcccagtctctcctgtactccatcgggtacaattatctggattggtatctgcagaagtctggccaatccccccagctgctgatctacctgggctccaacagagcaagcggcgtgcccgatagattcagcggcagcgggagcggcactgattttactctgaagatcagcagggtggaggccgaagatgtgggattttactactgcatgcaagcactgcagactccttacacattcggccagggaactaagctggagatcaaaagaaccgtggcagctccaagcgtcttcattttcccaccttctgacgagcagctgaagtccggcacagcttccgtcgtgtgcctcctgaacaacttctaccccagggaggcaaaggtgcaatggaaagtggacaacgctctgcagagcggaaacagtcaggagtccgtgaccgagcaggacagcaaagactccacttacagcctgagctctactctgaccctgagcaaagctgactacgagaagcataaggtgtatgcttgcgaggtcacccaccagggcctctcttctcccgtgaccaagagcttcaacagaggcgagtgctga

SEQ ID NO: 17 - DNA, 759 bp, synthetic construct
  misc_feature 1..759: note = source = /note="Description of Artificial Sequence: Syntheticpolynucleotide"
  source 1..759: mol_type = other DNA
atggcaactggaagcaggacctccctgctcctggctttcggcctgctctgtctgccatggctgcaagaaggatctgcctttcctacaattccactgtccgacatcgtgatgacacagtcccccctgtctctgcctgtcaccccaggcgaaccagcctctatttcttgtcggtcctctcagtccctgctgtatagcatcggatataattatctggactggtacctgcaaaaatccggccagtctcctcagctgctgatctatctgggctccaaccgggctagcggagtcccagaccggttttccgggtctggcagtgggacagattttacactgaaaatttcccgggtggaggctgaggacgtgggattttactactgtatgcaggccctgcaaaccccatatactttcggacagggaacaaagctggagatcaaaagaaccgtggccgcccccagcgttttcatcttcccaccaagcgacgagcagctcaaatctgggaccgctagcgtggtctgtctgctgaataacttctacccaagggaagcaaaggtgcagtggaaggtcgacaacgcactgcagagcgggaactcccaggagagcgtgactgaacaggacagcaaggacagcacctatagcctcagcagcactctgaccctgtctaaagctgattacgaaaaacacaaggtgtatgcttgtgaagtgactcaccagggcctgtcttcccctgttacaaagtccttcaatagaggagaatgttaa

SEQ ID NO: 18 - protein, 446 aa, synthetic construct
  REGION 1..446: note = source = /note="Description of Artificial Sequence: Syntheticpolypeptide"
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTYLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 214 aa, synthetic construct
  REGION 1..214: note = source = /note="Description of Artificial Sequence: Syntheticpolypeptide"
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 451 aa, synthetic construct
  REGION 1..451: note = source = /note="Description of Artificial Sequence: Syntheticpolypeptide"
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 219 aa, synthetic construct
  REGION 1..219: note = source = /note="Description of Artificial Sequence: Syntheticpolypeptide"
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 479 aa, synthetic construct
  REGION 1..479: note = source = /note="Description of Artificial Sequence: Syntheticpolypeptide"
MATGSRTSLLLAFGLLCLPWLQEGSAFPTIPLSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVTYLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 247 aa, synthetic construct
  REGION 1..247: note = source = /note="Description of Artificial Sequence: Syntheticpolypeptide"
MATGSRTSLLLAFGLLCLPWLQEGSAFPTIPLSDIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 484 aa, synthetic construct
  REGION 1..484: note = source = /note="Description of Artificial Sequence: Syntheticpolypeptide"
MATGSRTSLLLAFGLLCLPWLQEGSAFPTIPLSEVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 252 aa, synthetic construct
  REGION 1..252: note = source = /note="Description of Artificial Sequence: Syntheticpolypeptide"
MATGSRTSLLLAFGLLCLPWLQEGSAFPTIPLSDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 33 aa, synthetic construct
  REGION 1..33: note = source = /note="Description of Artificial Sequence: Syntheticpolypeptide"
MATGSRTSLLLAFGLLCLPWLQEGSAFPTIPLS

SEQ ID NO: 27 - DNA, 140 bp, synthetic construct
  misc_feature 1..140: note = source = /note="Description of Artificial Sequence: Syntheticpolynucleotide"
  source 1..140: mol_type = other DNA
ggacagatcgcctggagacgccatccacgctgttttgacctccatagaagacaccgggaccgatccagcctccgcggccgggaacggtgcattggaacgcggattccccgtgccaagagtgactcaccgtccttgacacg

SEQ ID NO: 28 - DNA, 100 bp, synthetic construct
  misc_feature 1..100: note = source = /note="Description of Artificial Sequence: Syntheticpolynucleotide"
  source 1..100: mol_type = other DNA
cgggtggcatccctgtgacccctccccagtgcctctcctggccctggaagttgccactccagtgcccaccagccttgtcctaataaaattaagttgcatc

SEQ ID NO: 29 - protein, 6 aa, synthetic construct
  REGION 1..6: note = source = /note="Description of Artificial Sequence: Synthetic6xHis tag"
HHHHHH